(19) United States
(12) Patent Application Publication
(10) Pub. No.: US 2009/0286938 A1
(43) Pub. Date: Nov. 19, 2009

(54) SYNTHESIS OF HYBRID BLOCK COPOLYMERS FROM DIFLUOROACETATE AMMONIUM SALTS

(75) Inventors: Kevin N. Sill, Tampa, FL (US); Gregoire Cardoen, Tampa, FL (US); Kurt Breitenkamp, San Diego, CA (US)

Correspondence Address: CHOATE, HALL & STEWART LLP, TWO INTERNATIONAL PLACE, BOSTON, MA, 02110, US

(73) Assignee: Intezyne Technologies, Inc., Tampa, FL (US)

(21) Appl. No.: 12/433,176

(22) Filed: Apr. 30, 2009

Related U.S. Application Data
(60) Provisional application No. 61/049,320, filed on Apr. 30, 2008.

(51) Int. Cl. C08G 69/48; C07C 53/21

(52) U.S. Cl. 525/419; 562/605

(57) ABSTRACT

The present invention provides polymerization initiators and uses thereof.

### CROSS-REFERENCE TO RELATED APPLICATIONS

The present application claims priority to U.S. Provisional patent application Ser. No. 61/049,320, filed Apr. 30, 2008, the entirety of which is hereby incorporated herein by reference.

### FIELD OF THE INVENTION

The present invention relates to the field of polymer chemistry and more particularly to block copolymers, uses thereof, and intermediates thereto.

### BACKGROUND OF THE INVENTION

Multi-block copolymers comprising a synthetic polymer portion and a poly(amino acid) portion are of great synthetic interest. The poly(amino acid) portion of such polymers is typically prepared by the ring-opening polymerization of an amino acid-N-carboxy-anhydride (NCA). However, methods for preparing the poly(amino acid) block that employ free amines as initiators of the NCA polymerization afford block copolymers with a wide range of polydispersity indices (PDIs) that tend to be quite high. For example, Schlaad reported PDI values of 1.12-1.60 by initiating polymerization with amino-terminated polystyrene. Schlaad (2003 Eur. Chem. J.) also reports a PDI of 7.0 for crude PEG-b-poly(L-benzyl glutamate) copolymers and a PDI of 1.4 after fractionation. Chen (Biomaterials, 2004) reported a PDI of 1.5 for poly(ε-caprolactone) (PCL)-b-poly(ethylene glycol) (PEG)-b-poly (γ-benzyl-L-glutamate)(PBLG). It is believed that these high PDIs are due to the highly reactive nature of the NCAs.

To date, the only reported synthetic methods to prepare multi-block copolymers that contain a poly(amino acid) portion with a narrower distribution of molecular weights, is amine-initiated NCA polymerization utilizing high vacuum techniques developed by Hadjichristidis (Biomacromolecules, 2004), and the nickel-catalyzed coordination-insertion polymerization of NCAs developed by Deming (see U.S. Pat. No. 6,686,446). Poly(amino acids) synthesized using high vacuum techniques are synthetically challenging to prepare, employ handmade reaction vessels, and require long time periods for reagent purification and complete polymerization to be achieved. Due to these factors, only a few grams of poly(amino acid) can be prepared in a single polymerization reaction. In addition, since multi-block copolymers that comprise a poly(amino acid) portion are typically designed for biological applications, the use of organometallic initiators and catalysts is undesirable.

Accordingly, there remains a need for a method for preparing block copolymers having a synthetic polymer portion and a poly(amino acid) portion wherein the method is well controlled and multiple poly(amino acid) blocks are incorporated.

### BRIEF DESCRIPTION OF THE DRAWINGS

FIG. 1 depicts the GPC chromatogram of N₃-PEG12K-b-Poly(Asp(But)₁₀)-b-Poly(d-Leu₂₀-co-Tyr(Bzl)₂₀ prepared from N₃-PEG12K-NH₃ DFA salt (Example 18).

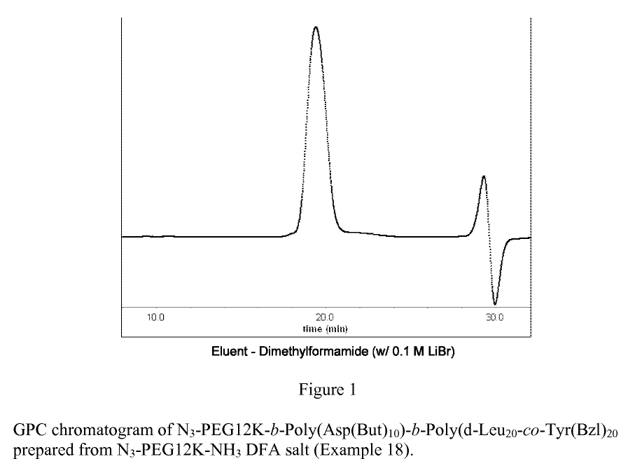

FIG. 2 depicts the GPC chromatogram of N₃-PEG12K-b-Poly(Asp(But)₁₀)-b-Poly(d-Leu₂₀-co-Tyr(Bzl)₂₀ prepared from N₃-PEG12K-NH₃HCl salt (Example 20).

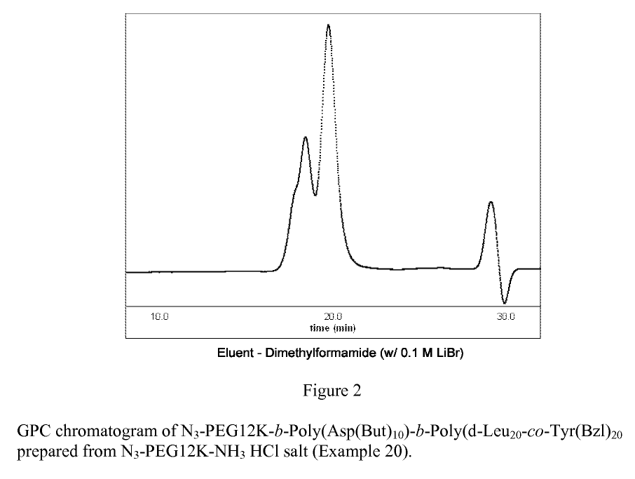

FIG. 3 depicts GPC chromatogram of N₃-PEG12K-b-Poly(Asp(But)₁₀)-b-Poly(d-Leu₂₀-co-Tyr(Bzl)₂₀ prepared from N₃-PEG12K-NH₃HCl salt (Example 21).

FIG. 4 depicts the polymerization kinetics of N₃-PEG12K-b-Poly(Asp(O^{t}Bu)₁₀)-b-Poly(D-Leu₂₀-co-Tyr(OBzl)₂₀)-Ac from N₃-PEG12K-NH₂ with different salts.

FIG. 5 depicts the polymerization kinetics of N₃-PEG12K-b-Poly(Asp(O^{t}Bu)₁₀)-b-Poly(D-Leu₂₀-co-Tyr(OBzl)₂₀)-Ac from N₃-PEG12K-NH₂ with different salts.

### DETAILED DESCRIPTION OF CERTAIN EMBODIMENTS OF THE INVENTION

### 1. General Description

A method for the controlled polymerization of an NCA, initiated by a polystyrene amine salt, was first reported by Schlaad and coworkers (Chem. Comm., 2003, 2944-2945). It is believed that, during the reaction, the chain end exists primarily in its unreactive salt form as a dormant species and that the unreactive amine salt is in equilibrium with the reactive amine. The free amine is capable of ring opening the NCA, which adds one repeat unit to the polymer chain. This cycle repeats until all of the monomer is consumed and the final poly(amino acid) is formed. This reported method has limitations in that only a single poly(amino acid) block is incorporated. In addition, this reported method only described the use of a polystyrene macroinitiator. In another publication by Schlaad and coworkers (Eur. Phys. J., 2003, 10, 17-23), the author indicates that use of a PEG macroinitiator results in diverse and unpredictable PDIs. The author further indicates that even “the coupling of preformed polymer segments like that of a haloacylated poly(ethylene oxide) with poly(L-aspartic acid) . . . yields block copolymers that are chemically disperse and are often contaminated with homopolymers.”

The present invention provides methods for the synthesis of block copolymers containing one or more poly(amino acid) blocks and one synthetic polymer block comprising poly(ethylene glycol). The poly(amino acid) portions of these block copolymers are prepared by controlled ring-opening polymerization of N-carboxyanhydrides (“NCA's”) wherein said polymerization is initiated by an ammonium difluoroacetate (“DFA”) salt. The amine salt initiators provided herein, and used in methods of the present invention, are poly(ethylene glycol)s with terminal amine DFA salts (referred to herein as “macroinitiators”). Without wishing to be bound by any particular theory, it is believed that use of a provided DFA amine salt reduces or eliminates many side reactions that are commonly observed with traditional polymerization of these reactive monomers. This leads to block copolymers with narrow distributions of block lengths and molecular weights.

Breitenkamp, et al, described the use of amine salt initiators for controlled ring-opening polymerization of N-carboxyanhydrides (see United States patent application publication number 20060172914, published Aug. 3, 2006). While evaluating the performance of various ammonium salts, it was surprisingly found that the nature of the counter ion has a profound effect on the kinetics and efficiency of the reaction. For example, an ammonium trifluoroacetate macroinitiator is capable of copolymerizing lysine (Z) NCA with leucine NCA, but is incapable of homopolymerizing lysine(Z) NCA. In contrast, lysine (Z) NCA can be homopolymerized through the use of an ammonium hydrochloride macroinitiator. While the hydrochloride salt is more versatile in terms of the variety of monomers that can be polymerized, the polymerization must be run at 80° C. for an acceptable rate of polymerization. Depending on the chemical functionality of the macroinitiator, such higher temperatures required for the ammonium hydrochloride macroinitiator can lead to an increase in side reactions, especially in the case of azide functionalized macroinitiators. However, while the trifluoracetate salt is less versatile, it provides a much higher rate of polymerization when run at 60° C., and lowers the probability of side reactions.

Surprisingly, it was found that difluoroacetate ammonium salts are effective macroinitiators for the polymerization of NCA's. Such difluoroacetate ammonium salts are effective at homopolymerizing and copolymerizing a wide range of NCA's. In addition, it was found that an optimum polymerization temperature is 60° C., where the polymerization rate is 2-3 times higher than observed for the corresponding ammonium hydrochloride salt at 80° C. This lower polymerization temperature limits the possibility of side reactions thereby producing a purer product. In addition, use of DFA is more amenable to sensitive functional groups. Without wishing to be bound by any particular theory, it is believed that this is due to the fact that DFA is a weaker organic acid than trifluoroacetic acid, and milder than mineral acids such as hydrochloric acid. It is also believed that the use of a weaker organic acid allows for a more dynamic equilibrium between the dormant ammonium salt and the active amine.

In certain embodiments, the PEG block possesses a molecular weight of approx. 10,000 Da (225 repeat units) and contains at least one terminal ammonium salt used to initiate the synthesis of poly(amino acid) multi-block copolymers. In other embodiments, the PEG block possesses a molecular weight of approx. 12,000 Da (270 repeat units) and contains at least one terminal ammonium salt used to initiate the synthesis of poly(amino acid) multi-block copolymers. In yet other embodiments, the PEG block possesses a molecular weight of approx. 8,000 Da (180 repeat units) and contains at least one terminal ammonium salt used to initiate the synthesis of poly(amino acid) multi-block copolymers. In another embodiment, the PEG block possesses a molecular weight of approx. 5,000 Da (110 repeat units) and contains at least one terminal ammonium salt used to initiate the synthesis of poly(amino acid) multi-block copolymers. In certain embodiments, the PEG block possesses a molecular weight of approx. 20,000 Da (454 repeat units) and contains at least one terminal ammonium salt used to initiate the synthesis of poly(amino acid) multi-block copolymers. In yet other embodiments, the PEG block possesses a molecular weight of approx. 40,000 Da (908 repeat units) and contains at least one terminal ammonium salt used to initiate the synthesis of poly(amino acid) multi-block copolymers. Without wishing to be bound by theory, it is believed that this particular PEG chain length imparts adequate water-solubility to the micelles and provides relatively long in vivo circulation times.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “sequential polymerization”, and variations thereof, refers to the method wherein, after a first monomer (e.g. NCA, lactam, or imide) is incorporated into the polymer, thus forming an amino acid “block”, a second monomer (e.g. NCA, lactam, or imide) is added to the reaction to form a second amino acid block, which process may be continued in a similar fashion to introduce additional amino acid blocks into the resulting multi-block copolymers.

As used herein, the term “block copolymer” refers to a polymer comprising at least one synthetic polymer portion and at least one poly(amino acid) portion. The term “multi-block copolymer” refers to a polymer comprising at least one synthetic polymer and two or more poly(amino acid) portions. These are also referred to as triblock copolymers (having two poly(amino acid) portions), tetrablock copolymers (having three poly(amino acid portions), etc. Such multi-block copolymers include those having the format X-W-X, X-W-X′, W-X-X′, W-X-X′-X″, X′-X-W-X-X′, X′-X-W-X″-X′″, or W-X-X′-X wherein W is a synthetic polymer portion and X, X′, X″, and X′″ are poly(amino acid) chains or “amino acid blocks”. In certain aspects, the synthetic polymer is used as the center block which allows the growth of multiple blocks symmetrically from the center.

As used herein, the term “portion” or “block” refers to a repeating polymeric sequence of defined composition. A portion or a block may consist of a single monomer or may be comprise of on or more monomers, resulting in a “mixed block”.

One skilled in the art will recognize that a monomer repeat unit is defined by parentheses around the repeating monomer unit. The number (or letter representing a numerical range) on the lower right of the parentheses represents the number of monomer units that are present in the polymer chain. In the case where only one monomer represents the block (e.g. a homopolymer), the block will be denoted solely by the parentheses. In the case of a mixed block, multiple monomers comprise a single, continuous block. It will be understood that brackets will define a portion or block. For example, one block may consist of four individual monomers, each defined by their own individual set of parentheses and number of repeat units present. All four sets of parentheses will be enclosed by a set of brackets, denoting that all four of these monomers combine in random, or near random, order to comprise the mixed block. For clarity, the randomly mixed block of [BCADDCBADABCDABC] would be represented in shorthand by [(A)₄(B)₄(C)₄(D)₄].

As used herein, the term “synthetic polymer” refers to a polymer that is not a poly(amino acid). Such synthetic polymers are well known in the art and include polystyrene, polyalkylene oxides, such as poly(ethylene oxide) (also referred to as PEO, polyethylene glycol or PEG), and derivatives thereof.

As used herein, the term “poly(amino acid)” or “amino acid block” refers to a covalently linked amino acid chain wherein each monomer is an amino acid unit. Such amino acid units include natural and unnatural amino acids. In certain embodiments, each amino acid unit is in the L-configuration. Such poly(amino acids) include those having suitably protected functional groups. For example, amino acid monomers may have hydroxyl or amino moieties which are optionally protected by a suitable hydroxyl protecting group or a suitable amine protecting group, as appropriate. Such suitable hydroxyl protecting groups and suitable amine protecting groups are described in more detail herein, infra. As used herein, an amino acid block comprises one or more monomers or a set of two or more monomers. In certain embodiments, an amino acid block comprises one or more monomers such that the overall block is hydrophilic. In other embodiments, an amino acid block comprises one or more monomers such that the overall block is hydrophobic. In still other embodiments, amino acid blocks of the present invention include random amino acid blocks, ie blocks comprising a mixture of amino acid residues.

As used herein, the phrase “natural amino acid side-chain group” refers to the side-chain group of any of the 20 amino acids naturally occurring in proteins. Such natural amino acids include the nonpolar, or hydrophobic amino acids, glycine, alanine, valine, leucine isoleucine, methionine, phenylalanine, tryptophan, and proline. Cysteine is sometimes classified as nonpolar or hydrophobic and other times as polar. Natural amino acids also include polar, or hydrophilic amino acids, such as tyrosine, serine, threonine, aspartic acid (also known as aspartate, when charged), glutamic acid (also known as glutamate, when charged), asparagine, and glutamine. Certain polar, or hydrophilic, amino acids have charged side-chains. Such charged amino acids include lysine, arginine, and histidine. One of ordinary skill in the art would recognize that protection of a polar or hydrophilic amino acid side-chain can render that amino acid nonpolar. For example, a suitably protected tyrosine hydroxyl group can render that tyroine nonpolar and hydrophobic by virtue of protecting the hydroxyl group.

As used herein, the phrase “unnatural amino acid side-chain group” refers to amino acids not included in the list of 20 amino acids naturally occurring in proteins, as described above. Such amino acids include the D-isomer of any of the 20 naturally occurring amino acids. Unnatural amino acids also include homoserine, ornithine, and thyroxine. Other unnatural amino acids side-chains are well know to one of ordinary skill in the art and include unnatural aliphatic side chains. Other unnatural amino acids include modified amino acids, including those that are N-alkylated, cyclized, phosphorylated, acetylated, amidated, labeled, and the like.

As used herein, the phrase “living polymer chain-end” refers to the terminus resulting from a polymerization reaction which maintains the ability to react further with additional monomer or with a polymerization terminator.

As used herein, the term “termination” refers to attaching a terminal group to a polymer chain-end by the reaction of a living polymer with an appropriate compound. Alternatively, the term “termination” may refer to attaching a terminal group to an amine or hydroxyl end, or derivative thereof, of the polymer chain.

As used herein, the term “polymerization terminator” is used interchangeably with the term “polymerization terminating agent” and refers to a compound that reacts with a living polymer chain-end to afford a polymer with a terminal group. Alternatively, the term “polymerization terminator” may refer to a compound that reacts with an amine or hydroxyl end, or derivative thereof, of the polymer chain, to afford a polymer with a terminal group.

As used herein, the term “polymerization initiator” refers to a compound, which reacts with, or whose anion or free base form reacts with, the desired monomer in a manner which results in polymerization of that monomer. In certain embodiments, the polymerization initiator is the compound that reacts with an alkylene oxide to afford a polyalkylene oxide block. In other embodiments, the polymerization initiator is the amine salt described herein.

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-20 carbon atoms. In some embodiments, aliphatic groups contain 1-10 carbon atoms. In other embodiments, aliphatic groups contain 1-8 carbon atoms. In still other embodiments, aliphatic groups contain 1-6 carbon atoms, and in yet other embodiments aliphatic groups contain 1-4 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon. This includes any oxidized form of nitrogen, sulfur, phosphorus, or silicon; the quaternized form of any basic nitrogen, or; a substitutable nitrogen of a heterocyclic ring including ═N— as in 3,4-dihydro-2H-pyrrolyl, —NH— as in pyrrolidinyl, or ═N(R^{†})— as in N-substituted pyrrolidinyl.

The term “unsaturated”, as used herein, means that a moiety has one or more units of unsaturation.

The term “aryl” used alone or as part of a larger moiety as in “aralkyl”, “aralkoxy”, or “aryloxyalkyl”, refers to monocyclic, bicyclic, and tricyclic ring systems having a total of five to fourteen ring members, wherein at least one ring in the system is aromatic and wherein each ring in the system contains three to seven ring members. The term “aryl” may be used interchangeably with the term “aryl ring”.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}; —N(R^{◯})C(S)NR^{◯}; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR—, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯}) S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{}, —(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. A suitable tetravalent substituent that is bound to vicinal substitutable methylene carbons of an “optionally substituted” group is the dicobalt hexacarbonyl cluster represented by

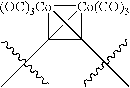

when depicted with the methylenes which bear it.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Protected hydroxyl groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino(-NHBOC), ethyloxycarbonylamino, methyloxycarbonylamino, trichloroethyloxycarbonylamino, allyloxycarbonylamino(-NHAlloc), benzyloxocarbonylamino(-NHCBZ), allylamino, benzylamino(-NHBn), fluorenylmethylcarbonyl(-NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl)acetal, 1,3-dioxanes, 1,3-dioxolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

A “crown ether moiety” is the radical of a crown ether. A crown ether is a monocyclic polyether comprised of repeating units of —CH₂CH₂O—. Examples of crown ethers include 12-crown-4,15-crown-5, and 18-crown-6.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

As used herein, the term “detectable moiety” is used interchangeably with the term “label” and relates to any moiety capable of being detected (e.g., primary labels and secondary labels). A “detectable moiety” or “label” is the radical of a detectable compound.

“Primary” labels include radioisotope-containing moieties (e.g., moieties that contain ³²P, ³³P, ³⁵S, or ¹⁴C), mass-tags, and fluorescent labels, and are signal-generating reporter groups which can be detected without further modifications.

Other primary labels include those useful for positron emission tomography including molecules containing radioisotopes (e.g. ¹⁸F) or ligands with bound radioactive metals (e.g. ⁶²Cu). In other embodiments, primary labels are contrast agents for magnetic resonance imaging such as gadolinium, gadolinium chelates, or iron oxide (e.g Fe₃O₄ and Fe₂O₃) particles. Similarly, semiconducting nanoparticles (e.g. cadmium selenide, cadmium sulfide, cadmium telluride) are useful as fluorescent labels. Other metal nanoparticles (e.g colloidal gold) also serve as primary labels.

“Secondary” labels include moieties such as biotin, or protein antigens, that require the presence of a second compound to produce a detectable signal. For example, in the case of a biotin label, the second compound may include streptavidin-enzyme conjugates. In the case of an antigen label, the second compound may include an antibody-enzyme conjugate. Additionally, certain fluorescent groups can act as secondary labels by transferring energy to another compound or group in a process of nonradiative fluorescent resonance energy transfer (FRET), causing the second compound or group to then generate the signal that is detected.

Unless otherwise indicated, radioisotope-containing moieties are optionally substituted hydrocarbon groups that contain at least one radioisotope. Unless otherwise indicated, radioisotope-containing moieties contain from 1-40 carbon atoms and one radioisotope. In certain embodiments, radioisotope-containing moieties contain from 1-20 carbon atoms and one radioisotope.

The terms “fluorescent label”, “fluorescent group”, “fluorescent compound”, “fluorescent dye”, and “fluorophore”, as used herein, refer to compounds or moieties that absorb light energy at a defined excitation wavelength and emit light energy at a different wavelength. Examples of fluorescent compounds include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The term “substrate”, as used herein refers to any material or macromolecular complex to which a functionalized end-group of a block copolymer can be attached. Examples of commonly used substrates include, but are not limited to, glass surfaces, silica surfaces, plastic surfaces, metal surfaces, surfaces containing a metallic or chemical coating, membranes (eg., nylon, polysulfone, silica), micro-beads (eg., latex, polystyrene, or other polymer), porous polymer matrices (eg., polyacrylamide gel, polysaccharide, polymethacrylate), macromolecular complexes (eg., protein, polysaccharide).

### 3. Description of Exemplary Embodiments

As described generally above, one aspect of the present invention provides a method for preparing a multi-block copolymer comprising one or more poly(amino acid) blocks and one or more synthetic polymer blocks, wherein said method comprises the steps of sequentially polymerizing one or more cyclic amino acid monomers onto a synthetic polymer having a terminal amine difluoroacetic acid salt wherein said polymerization is initiated by said amine difluoroacetic acid salt. In certain embodiments, said polymerization occurs by ring-opening polymerization of the cyclic amino acid monomers. In other embodiments, the cyclic amino acid monomer is an amino acid NCA, lactam, or imide.

As described generally above, the synthetic polymers used in methods of the present invention have a terminal amine difluoroacetic acid salt for initiating the polymerization of a cyclic amino acid monomer. Such salts include the acid addition salts of an amino group formed with difluoroacetic acid.

As described generally above, the synthetic polymers used in methods of the present invention have a terminal amine difluoroacetic acid salt. In certain embodiments, the synthetic polymer is poly(ethylene glycol) (PEG) having a terminal amine DFA salt (“PEG macroinitiator”) which initiates the polymerization of NCAs to provide PEG-poly(amino acid) multi-block copolymers. Such synthetic polymers having a terminal amine DFA salt may be prepared from synthetic polymers having a terminal amine. Such synthetic polymers having a terminal amine group are known in the art and include PEG-amines. PEG-amines may be obtained by the deprotection of a suitably protected PEG-amine. Preparation of such suitably protected PEG-amines, and methods of deprotecting the same, is described in detail in U.S. patent application Ser. No. 11/256,735, filed Oct. 24, 2005 and published on Jun. 29, 2006 as US 20060142506, the entirety of which is hereby incorporated herein by reference.

As described in US 20060142506, suitably protected PEG-amines may be formed by terminating the living polymer chain end of a PEG with a terminating agent that contains a suitably protected amine. The suitably protected amine may then be deprotected to generate a PEG that is terminated with a free amine that may subsequently be converted into the corresponding PEG-amine salt macroinitiator. In certain embodiments, the PEG-amine salt macroinitiator of the present invention is prepared directly from a suitably protected PEG-amine by deprotecting said protected amine with an acid. Accordingly, in other embodiments, the terminating agent has suitably protected amino group wherein the protecting group is acid-labile.

Alternatively, suitable synthetic polymers having a terminal amine DFA salt may be prepared from synthetic polymers that contain terminal functional groups that may be converted to amine DFA salts by known synthetic routes. In certain embodiments, the conversion of the terminal functional groups to the amine DFA salts is conducted in a single synthetic step. In other embodiments, the conversion of the terminal functional groups to the amine DFA salts is achieved by way of a multi-step sequence. Functional group transformations that afford amines, amine salts, or protected amines are well known in the art and include those described in Larock, R. C., “Comprehensive Organic Transformations,” John Wiley & Sons, New York, 1999.

Alternatively, and as described in detail in US 20060142506, suitably protected PEG-amines may be formed by initiating the polymerization of ethylene oxide with a compound that contains a suitably protected amino moiety. The PEG formed therefrom may be terminated by any manner known in the art, including those described in US 20060142506. The method of termination may incorporate a additional suitably protected amine functional group, or a precursor thereto, such that each terminus of the PEG formed therefrom may be subsequently converted to an amine DFA salt that may be employed in the polymerization of the cyclic monomers described herein. In certain embodiments, only one terminus of such a PEG is converted to an amine DFA salt that is then employed in the formation of one or more poly(amino acid) blocks. Following such polymerizations, the amine DFA salt terminus may be converted to an unreactive form, and then the other terminus may be converted to an amine DFA salt for use in the introduction of additional poly(amino acid) blocks.

One of ordinary skill in the art would recognize that the embodiments described above and herein that employ PEG as the synthetic polymer block can be readily applied to other synthetic polymers. Therefore, this invention contemplates multiblock copolymers of the permutations described herein that employ synthetic polymers other than PEG. In certain embodiments, the synthetic polymer block is polypropylene oxide (PPO), PEG-PPO-PEG block copolymers (Pluronics®), polyesters, polyamides, poly(ethylene imine), polyphosphazines, polyacrylates, or polymethacrylates.

In certain embodiments, the synthetic polymer is poly(ethylene glycol) (PEG) having one or two terminal amine DFA salt (s) (“PEG macroinitiator”) to initiate the polymerization of NCAs to provide a PEG-poly(amino acid) multi-block copolymer as illustrated in Scheme 1, below.

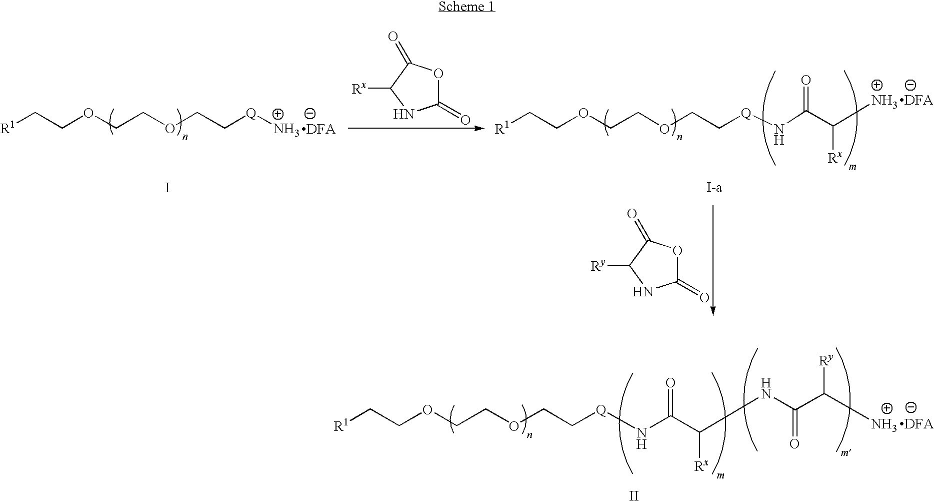

Scheme 1 above depicts a polymerization method of the present invention. A macroinitiator of formula I, described in detail below, is treated with a first amino acid NCA to form a compound of formula I-a having a first amino acid block. The second amino acid NCA is added to the living polymer of formula I-a to form a compound of formula II having two differing amino acid blocks. Each of the R¹, n, Q, R^{x}, R^{y}, m, and m′ groups depicted in Scheme 1 are as defined and described in classes and subclasses, singly and in combination, herein.

Another aspect of the present invention provides a method of for preparing a multi-block copolymer comprising two or more different poly(amino acid) blocks and a PEG synthetic polymer block, wherein said method comprises the steps of:
(a) providing a compound of formula I:

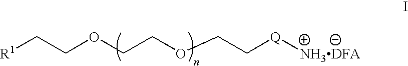

wherein:
   n is 10-2500;
   R¹1 is -Z(CH₂CH₂Y)ₚ(CH₂)ₜR³, wherein:
      Z is —O—, —S—, —C≡C—, or —CH₂—;
      each Y is independently —O— or —S—;
      p is 0-10;
      t is 0-10; and
      R³ is —N₃, —CN, a mono-protected amine, a di-protected amine, a protected aldehyde, a protected hydroxyl, a protected carboxylic acid, a protected thiol, a 9-30-membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety; and
   Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
      -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
(b) polymerizing a first cyclic amino acid monomer onto the amine salt terminal end of formula I;
(c) optionally polymerizing a second cyclic amino acid monomer onto the living polymer end, wherein said second cyclic amino acid monomer is different from said first cyclic amino acid monomer; and
(d) optionally polymerizing additional cyclic amino acid monomers onto the living polymer end.

In certain embodiments, the cyclic amino acid monomers include N-carboxy anhydrides (NCAs), lactams, and cyclic imides. According to one embodiment, the cyclic amino acid monomer is an NCA. NCAs are well known in the art and are typically prepared by the carbonylation of amino acids by a modification of the Fuchs-Farthing method (Kricheldorf, α-Aminoacid-N-Carboxy-Anhydrides and Related Heterocycles: Syntheses, Properties, Peptide Synthesis, Polymerization, 1987). Although reaction conditions vary among different amino acids, most, if not all, natural and unnatural, 2-substituted amino acids can be converted to N-carboxy anhydrides using phosgene gas or triphosgene (for ease of handling). It will be appreciated that, although α-amino acids are described below, one of ordinary skill in the art would recognize that NCAs may be prepared from 0- and γ-amino acids as well. In addition, NCAs can be prepared from dimers or trimers of amino acids.

Both D and L NCA enantiomers can be synthesized and any combination of the two stereoisomers can undergo ring-opening polymerization. Advanced Chemtech (http://www.advancedchemtech.com) and Bachem (www.bachem.com) are commercial and widely-referenced sources for both protected and unprotected amino acids. It will be appreciated that amino acid dimers and trimers can form cyclic anhydrides and are capable of ROP in accordance with the present invention.

In certain embodiments, the cyclic amino acid monomer is a carboxylate-protected aspartic acid NCA, a hydroxyl-protected tyrosine NCA, or an amino-protected lysine NCA. In other embodiments, the cyclic amino acid monomer is a t-butyl protected aspartic acid NCA, a benzyl-protected tyrosine NCA, or a Z-protected lysine NCA.

As defined generally above, the n group of formula I is 10-2500. In certain embodiments, the present invention provides compounds of formula I, as described above, wherein n is about 225. In other embodiments, n is about 275. In other embodiments, n is about 350. In other embodiments, n is about 10 to about 40. In other embodiments, n is about 40 to about 60. In other embodiments, n is about 60 to about 90. In still other embodiments, n is about 90 to about 150. In other embodiments, n is about 150 to about 200. In still other embodiments, n is about 200 to about 250. In other embodiments, n is about 250 to about 300. In other embodiments, n is about 300 to about 375. In other embodiments, n is about 400 to about 500. In still other embodiments, n is about 650 to about 750. In certain embodiments, n is selected from 50±10. In other embodiments, n is selected from 80±10, 115±10, 180±10, 225±10, 275±10, 315±10, or 340±10.

In certain embodiments, the R³ moiety of the R¹ group of formula I is —N₃.

In some embodiments, the R³ moiety of the R¹ group of formula I is methyl.

In certain embodiments, the R³ moiety of the R¹ group of formula I is an acetylene.

In other embodiments, the R³ moiety of the R¹1 group of formula I is —CN.

In still other embodiments, the R³ moiety of the R¹ group of formula I is a mono-protected amine or a di-protected amine.

In certain embodiments, the R³ moiety of the R¹ group of formula I is an optionally substituted aliphatic group. Examples include t-butyl, 5-norbornene-2-yl, octane-5-yl, acetylenyl, trimethylsilylacetylenyl, triisopropylsilylacetylenyl, and t-butyldimethylsilylacetylenyl. In some embodiments, said R³ moiety is an optionally substituted alkyl group. In other embodiments, said R³ moiety is an optionally substituted alkynyl or alkenyl group. When said R³ moiety is a substituted aliphatic group, suitable substituents on R³ include CN, N₃, trimethylsilyl, triisopropylsilyl, t-butyldimethylsilyl, N-methyl propiolamido, N-methyl-4-acetylenylanilino, N-methyl-4-acetylenylbenzoamido, bis-(4-ethynyl-benzyl)-amino, dipropargylamino, di-hex-5-ynyl-amino, di-pent-4-ynyl-amino, di-but-3-ynyl-amino, propargyloxy, hex-5-ynyloxy, pent-4-ynyloxy, di-but-3-ynyloxy, N-methyl-propargylamino, N-methyl-hex-5-ynyl-amino, N-methyl-pent-4-ynyl-amino, N-methyl-but-3-ynyl-amino, 2-hex-5-ynyldisulfanyl, 2-pent-4-ynyldisulfanyl, 2-but-3-ynyldisulfanyl, and 2-propargyldisulfanyl. In certain embodiments, the R¹ group is 2-(N-methyl-N-(ethynylcarbonyl)amino)ethoxy, 4-ethynylbenzyloxy, or 2-(4-ethynylphenoxy)ethoxy.

In certain embodiments, the R³ moiety of the R¹ group of formula I is an optionally substituted aryl group. Examples include optionally substituted phenyl and optionally substituted pyridyl. When said R³ moiety is a substituted aryl group, suitable substituents on R³ include CN, N₃, NO₂, —CH₃, —CH₂N₃, —CH═CH₂, —C≡CH, Br, I, F, bis-(4-ethynyl-benzyl)-amino, dipropargylamino, di-hex-5-ynyl-amino, di-pent-4-ynyl-amino, di-but-3-ynyl-amino, propargyloxy, hex-5-ynyloxy, pent-4-ynyloxy, di-but-3-ynyloxy, 2-hex-5-ynyloxy-ethyldisulfanyl, 2-pent-4-ynyloxy-ethyldisulfanyl, 2-but-3-ynyloxy-ethyldisulfanyl, 2-propargyloxy-ethyldisulfanyl, bis-benzyloxy-methyl, [1,3]dioxolan-2-yl, and [1,3]dioxan-2-yl.

In other embodiments, the R³ moiety is an aryl group substituted with a suitably protected amino group. According to another aspect, the R³ moiety is phenyl substituted with a suitably protected amino group.

In other embodiments, the R³ moiety of the R¹ group of formula I is a protected hydroxyl group. In certain embodiments the protected hydroxyl of the R³ moiety is an ester, carbonate, sulfonate, allyl ether, ether, silyl ether, alkyl ether, arylalkyl ether, or alkoxyalkyl ether. In certain embodiments, the ester is a formate, acetate, proprionate, pentanoate, crotonate, or benzoate. Exemplary esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Exemplary carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Exemplary alkyl ethers include methyl, benzyl, p-methoxybenzyl, 3,4-dimethoxybenzyl, trityl, t-butyl, and allyl ether, or derivatives thereof. Exemplary alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Exemplary arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

In certain embodiments, the R³ moiety of the R¹ group of formula I is a mono-protected or di-protected amino group. In certain embodiments R³ is a mono-protected amine. In certain embodiments R³ is a mono-protected amine selected from aralkylamines, carbamates, allyl amines, or amides. Exemplary mono-protected amino moieties include t-butyloxycarbonylamino, ethyloxycarbonylamino, methyloxycarbonylamino, trichloroethyloxy-carbonylamino, allyloxycarbonylamino, benzyloxocarbonylamino, allylamino, benzylamino, fluorenylmethylcarbonyl, formamido, acetamido, chloroacetamido, dichloroacetamido, trichloroacetamido, phenylacetamido, trifluoroacetamido, benzamido, and t-butyldiphenylsilylamino. In other embodiments R³ is a di-protected amine. Exemplary di-protected amines include di-benzylamine, di-allylamine, phthalimide, maleimide, succinimide, pyrrole, 2,2,5,5-tetramethyl-[1,2,5]azadisilolidine, and azide. In certain embodiments, the R³ moiety is phthalimido. In other embodiments, the R³ moiety is mono- or di-benzylamino or mono- or di-allylamino. In certain embodiments, the R¹ group is 2-dibenzylaminoethoxy.

In other embodiments, the R³ moiety of the R¹ group of formula I is a protected aldehyde group. In certain embodiments the protected aldehydro moiety of R³ is an acyclic acetal, a cyclic acetal, a hydrazone, or an imine. Exemplary R³ groups include dimethyl acetal, diethyl acetal, diisopropyl acetal, dibenzyl acetal, bis(2-nitrobenzyl)acetal, 1,3-dioxane, 1,3-dioxolane, and semicarbazone. In certain embodiments, R³ is an acyclic acetal or a cyclic acetal. In other embodiments, R³ is a dibenzyl acetal.

In yet other embodiments, the R³ moiety of the R¹ group of formula I is a protected carboxylic acid group. In certain embodiments, the protected carboxylic acid moiety of R³ is an optionally substituted ester selected from C₁₋₆ aliphatic or aryl, or a silyl ester, an activated ester, an amide, or a hydrazide. Examples of such ester groups include methyl, ethyl, propyl, isopropyl, butyl, isobutyl, benzyl, and phenyl ester. In other embodiments, the protected carboxylic acid moiety of R³ is an oxazoline or an ortho ester. Examples of such protected carboxylic acid moieties include oxazolin-2-yl and 2-methoxy-[1,3]dioxin-2-yl. In certain embodiments, the R¹ group is oxazolin-2-ylmethoxy or 2-oxazolin-2-yl-1-propoxy.

According to another embodiments, the R³ moiety of the R¹ group of formula I is a protected thiol group. In certain embodiments, the protected thiol of R³ is a disulfide, thioether, silyl thioether, thioester, thiocarbonate, or a thiocarbamate. Examples of such protected thiols include triisopropylsilyl thioether, t-butyldimethylsilyl thioether, t-butyl thioether, benzyl thioether, p-methylbenzyl thioether, triphenylmethyl thioether, and p-methoxyphenyldiphenylmethyl thioether. In other embodiments, R³ is an optionally substituted thioether selected from alkyl, benzyl, or triphenylmethyl, or trichloroethoxycarbonyl thioester. In certain embodiments, R³ is —S—S-pyridin-2-yl, —S—SBn, —S—SCH₃, or —S—S(p-ethynylbenzyl). In other embodiments, R³ is —S—S-pyridin-2-yl. In still other embodiments, the R¹ group is 2-triphenylmethylsulfanyl-ethoxy.

In certain embodiments, the R³ moiety of the R¹ group of formula I is a crown ether. Examples of such crown ethers include 12-crown-4,15-crown-5, and 18-crown-6.

In still other embodiments, the R³ moiety of the R¹ group of formula I is a detectable moiety. According to one aspect of the invention, the R³ moiety of the R¹ group of formula I is a fluorescent moiety. Such fluorescent moieties are well known in the art and include coumarins, quinolones, benzoisoquinolones, hostasol, and Rhodamine dyes, to name but a few. Exemplary fluorescent moieties of the R³ group of R¹ include anthracen-9-yl, pyren-4-yl, 9-H-carbazol-9-yl, the carboxylate of rhodamine B, and the carboxylate of coumarin 343.

In certain embodiments, the R³ moiety of the R¹ group of formula I is a group suitable for Click chemistry. Click reactions tend to involve high-energy (“spring-loaded”) reagents with well-defined reaction coordinates, giving rise to selective bond-forming events of wide scope. Examples include the nucleophilic trapping of strained-ring electrophiles (epoxide, aziridines, aziridinium ions, episulfonium ions), certain forms of carbonyl reactivity (aldehydes and hydrazines or hydroxylamines, for example), and several types of cycloaddition reactions. The azide-alkyne 1,3-dipolar cycloaddition is one such reaction. Click chemistry is known in the art and one of ordinary skill in the art would recognize that certain R³ moieties of the present invention are suitable for Click chemistry.

Compounds of formula I having R³ moieties suitable for Click chemistry are useful for conjugating said compounds to biological systems or macromolecules such as proteins, viruses, and cells, to name but a few. The Click reaction is known to proceed quickly and selectively under physiological conditions. In contrast, most conjugation reactions are carried out using the primary amine functionality on proteins (e.g. lysine or protein end-group). Because most proteins contain a multitude of lysines and arginines, such conjugation occurs uncontrollably at multiple sites on the protein. This is particularly problematic when lysines or arginines are located around the active site of an enzyme or other biomolecule. Thus, another embodiment of the present invention provides a method of conjugating the R¹ group of a compound of formula I to a macromolecule via Click chemistry. Yet another embodiment of the present invention provides a macromolecule conjugated to a compound of formula I via the R¹ group.

As defined generally above, Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur. In certain embodiments, Q is a valence bond. In other embodiments, Q is a bivalent, saturated C₁₋₁₂ alkylene chain, wherein 0-6 methylene units of Q are independently replaced by -Cy-, —O—, —NH—, —S—, —OC(O)—, —C(O)O—, or —C(O)—, wherein -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

In certain embodiments, Q is -Cy- (i.e. a C₁ alkylene chain wherein the methylene unit is replaced by -Cy-), wherein -Cy- is an optionally substituted 5-8 membered bivalent, saturated, partially unsaturated, or aryl ring having 0-4 heteroatoms independently selected from nitrogen, oxygen, or sulfur. According to one aspect of the present invention, -Cy- is an optionally substituted bivalent aryl group. According to another aspect of the present invention, -Cy- is an optionally substituted bivalent phenyl group. In other embodiments, -Cy- is an optionally substituted 5-8 membered bivalent, saturated carbocyclic ring. In still other embodiments, -Cy- is an optionally substituted 5-8 membered bivalent, saturated heterocyclic ring having 1-2 heteroatoms independently selected from nitrogen, oxygen, or sulfur. Exemplary -Cy- groups include bivalent rings selected from phenyl, pyridyl, pyrimidinyl, cyclohexyl, cyclopentyl, or cyclopropyl.

After incorporating the poly (amino acid) block portions into the multi-block coploymer of the present invention resulting in a multi-block copolymer of the form W-X-X′, the other end-group functionality, corresponding to the R¹ moiety of formula I, can be used to attach targeting groups for cell specific delivery including, but not limited to, detectable moieties, such as fluorescent dyes, covalent attachment to surfaces, and incorporation into hydrogels. Alternatively, the R¹ moiety of formula I is bonded to a biomolecule, drug, cell, or other suitable substrate.

In certain embodiments, the present invention provides a compound of formula I:

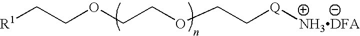

wherein each of R¹, n, and Q is as defined above and described in classes and subclasses singly and in combination.

In some embodiments, the present invention provides a method for preparing a compound of formula I:

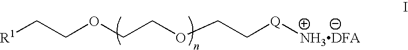

wherein each of R¹, n, and Q is as defined above and described in classes and subclasses singly and in combination, comprising the steps of:
(a) providing a compound of formula I-i:

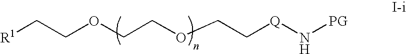

wherein PG is an acid-labile amino protecting group; and

(b) treating the compound of formula I-i with difluoroacetic acid to form the compound of formula I.

Suitable acid-labile amino protecting groups are well known in the art. In certain embodiments, the PG group of formula I-i is tert-butyloxycarbonyl (“BOC”) protecting group.

In certain embodiments, the present invention provides a method for preparing a compound of formula I:

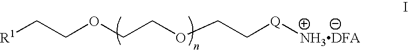

wherein each of R¹, n, and Q is as defined above and described in classes and subclasses singly and in combination, comprising the steps of:
(a) providing a compound of formula I-ii:

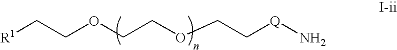

and

(b) treating the compound of formula I-ii with difluoroacetic acid to form the compound of formula I.

Exemplary compounds of formula I include:

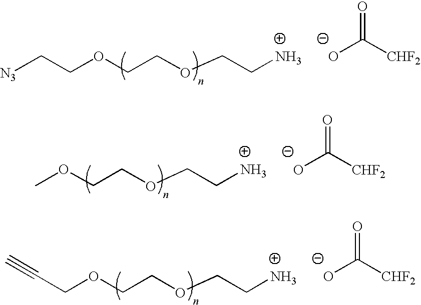

wherein each n is as defined above and described in classes and subclasses herein.

In some embodiments, the present invention provides a compound of formula I-a:

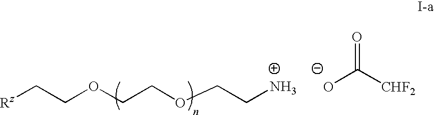

wherein R^{z} is CH₃O—, CH≡CCH₂O—, or N₃, and n is 10-2500.

In certain embodiments, the present invention provides a method for preparing a compound of formula I-a:

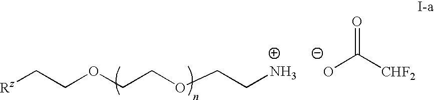

wherein R^{z} is CH₃O—, CH≡CCH₂O—, or N₃, and n is 10-2500;
comprising the steps of:
(a) providing a compound of formula

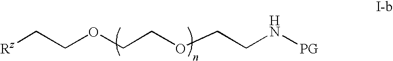

wherein PG is an acid-labile amino protecting group; and
(b) treating the compound of formula I-b with difluoroacetic acid to form a compound of formula I-a.

Suitable acid-labile amino protecting groups are well known in the art. In certain embodiments, the PG group of formula I-b is tert-butyloxycarbonyl (“BOC”) protecting group.

In certain embodiments, the present invention provides a method for preparing a compound of formula I-a:

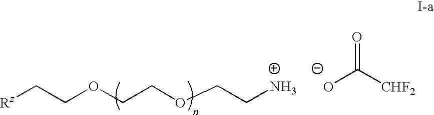

wherein R^{z} is CH₃O—, CH≡CCH₂O—, or N₃, and n is 10-2500;
comprising the steps of:
(a) providing a compound of formula

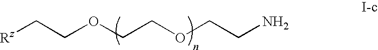

and
(b) treating the compound of formula I-c with difluoroacetic acid to form a compound of formula I-a.

In certain embodiments, difluoroacetic acid salts of the present invention are useful for preparing block copolymers of formula III:

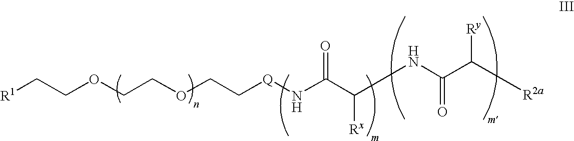

wherein:
n is 10-2500;
m is 0 to 1000;
m′ is 1 to 1000;
R^{x} is a natural or unnatural amino acid side-chain group that is capable of crosslinking;
R^{y} is a hydrophobic or ionic, natural or unnatural amino acid side-chain group;
R¹ is -Z(CH₂CH₂Y)ₚ(CH₂)ₜR³, wherein:
   Z is —O—, —S—, —C≡C—, or —CH₂—;
   each Y is independently —O— or —S—;
   p is 0-10;
   t is 0-10; and
R³ is hydrogen, —N₃, —CN, a mono-protected amine, a di-protected amine, a protected aldehyde, a protected hydroxyl, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
Q is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
R^{2a} is a mono-protected amine, a di-protected amine, —N(R⁴)₂, —NR⁴C(O)R⁴, —NR⁴C(O)N(R⁴)₂, —NR⁴C(O)OR⁴, or —NR⁴SO₂R⁴; and
each R⁴ is independently an optionally substituted group selected from hydrogen, aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
   two R⁴ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur.

Another aspect of the present invention provides a method for preparing a multi-block copolymer of formula II:

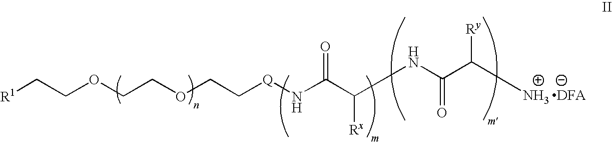

wherein:
n is 10-2500;
m is 0 to 1000;
m′ is 1 to 1000;
R^{x} is a natural or unnatural amino acid side-chain group that is capable of crosslinking;
R^{y} is a hydrophobic or ionic, natural or unnatural amino acid side-chain group;
R¹ is -Z(CH₂CH₂Y)ₚ(CH₂)ₜR³, wherein:
   Z is —O—, —S—, —C≡C—, or —CH₂—;
   each Y is independently —O— or —S—;
   p is 0-10;
   t is 0-10; and
R³ is hydrogen, —N₃, —CN, a mono-protected amine, a di-protected amine, a protected aldehyde, a protected hydroxyl, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
Q is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
      wherein said method comprises the steps of:
(a) providing a compound of formula I:

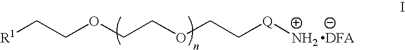

wherein:
   n is 10-2500;
   R¹ is -Z(CH₂CH₂Y)ₚ(CH₂)ₜR³, wherein:
      Z is —O—, —S—, —C≡C—, or —CH₂—;
      each Y is independently —O— or —S—;
      p is 0-10;
      t is 0-10; and
      R³ is —N₃, —CN, a mono-protected amine, a di-protected amine, a protected aldehyde, a protected hydroxyl, a protected carboxylic acid, a protected thiol, a 9-30-membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety; and
   Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
      -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
b) polymerizing a first cyclic amino acid monomer onto the amine salt terminal end of formula I, wherein said first cyclic amino acid monomer comprises R^{x}; and
(c) optionally polymerizing a second cyclic amino acid monomer, comprising R^{y}, onto the living polymer end, wherein said second cyclic amino acid monomer is different from said first cyclic amino acid monomer.

In some embodiments, the method further comprises the step of treating the compound of formula II with a suitable terminating agent to form a compound of formula III

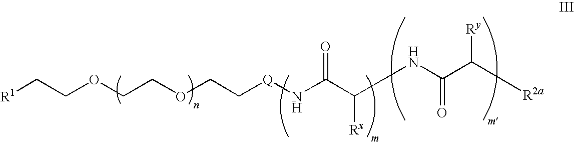

wherein each variable is as defined above and described herein.

In certain embodiments, the compound of formula I is a compound of formula I-a.

In certain embodiments, the preparation of formula II from formula I is performed at 25° C. to 100° C. In other embodiments, the reaction is performed at approximately 60° C. In yet other embodiments, the reaction is performed at 50° C. to 70° C.

As defined generally above, the n group of formula I, II, or III is 10-2500. In certain embodiments, the present invention provides compounds of formula I, II, or III, as described above, wherein n is about 225. In other embodiments, n is about 275. In other embodiments, n is about 350. In other embodiments, n is about 10 to about 40. In other embodiments, n is about 40 to about 60. In other embodiments, n is about 60 to about 90. In still other embodiments, n is about 90 to about 150. In other embodiments, n is about 150 to about 200. In still other embodiments, n is about 200 to about 250. In other embodiments, n is about 250 to about 300. In other embodiments, n is about 300 to about 375. In other embodiments, n is about 400 to about 500. In still other embodiments, n is about 650 to about 750. In certain embodiments, n is selected from 50±10. In other embodiments, n is selected from 80±10, 115±10, 180±10, 225±10, 275±10, 315±10, or 340±10.

According to another embodiment, the present invention provides a compound of formula I, II, or III, as described above, wherein said compound has a polydispersity index (“PDI”) of about 1.01 to about 1.2. According to another embodiment, the present invention provides a compound of formula I, II, or III, as described above, wherein said compound has a polydispersity index (“PDI”) of about 1.02 to about 1.05. According to yet another embodiment, the present invention provides a compound of formula I, II, or III, as described above, wherein said compound has a polydispersity index (“PDI”) of about 1.05 to about 1.10. In other embodiments, said compound has a PDI of about 1.01 to about 1.03. In other embodiments, said compound has a PDI of about 1.10 to about 1.15. In still other embodiments, said compound has a PDI of about 1.15 to about 1.20.

In certain embodiments, the m′ group of formula II or III is about 5 to about 500. In certain embodiments, the m′ group of formula II or III is about 10 to about 250. In other embodiments, m′ is about 10 to about 50. According to yet another embodiment, m′ is about 15 to about 40. In other embodiments, m′ is about 20 to about 40. According to yet another embodiment, m′ is about 50 to about 75. According to other embodiments, m and m′ are independently about 10 to about 100. In certain embodiments, m is 5-50. In other embodiments, m is 5-25. In certain embodiments, m′ is 5-50. In other embodiments, m′ is 5-10. In other embodiments, m′ is 10-20. In certain embodiments, m and m′ add up to about 30 to about 60. In still other embodiments, m is 1-20 repeat units and m′ is 10-50 repeat units.

In certain embodiments, the m group of formula II or III is zero, thereby forming a diblock copolymer.

In certain embodiments, R^{x} is a crosslinkable amino acid side-chain group and R^{y} is a hydrophobic amino acid side-chain group. Such crosslinkable amino acid side-chain groups include tyrosine, serine, cysteine, threonine, aspartic acid (also known as aspartate, when charged), glutamic acid (also known as glutamate, when charged), asparagine, histidine, lysine, arginine, and glutamine. Such hydrophobic amino acid side-chain groups include a suitably protected tyrosine side-chain, a suitably protected serine side-chain, a suitably protected threonine side-chain, phenylalanine, alanine, valine, leucine, tryptophan, proline, benzyl and alkyl glutamates, or benzyl and alkyl aspartates or mixtures thereof. In other embodiments, R^{y} is an ionic amino acid side-chain group. Such ionic amino acid side chain groups includes a lysine side-chain, arginine side-chain, or a suitably protected lysine or arginine side-chain, an aspartic acid side chain, glutamic acid side-chain, or a suitably protected aspartic acid or glutamic acid side-chain. One of ordinary skill in the art would recognize that protection of a polar or hydrophilic amino acid side-chain can render that amino acid nonpolar. For example, a suitably protected tyrosine hydroxyl group can render that tyrosine nonpolar and hydrophobic by virtue of protecting the hydroxyl group. Suitable protecting groups for the hydroxyl, amino, and thiol, and carboylate functional groups of R^{x} and R^{y} are as described herein.

In other embodiments, R^{y} comprises a mixture of hydrophobic and hydrophilic amino acid side-chain groups such that the overall poly(amino acid) block comprising R^{y} is hydrophobic. Such mixtures of amino acid side-chain groups include phenylalanine/tyrosine, phenalanine/serine, leucine/tyrosine, and the like. According to another embodiment, R^{y} is a hydrophobic amino acid side-chain group selected from phenylalanine, alanine, or leucine, and one or more of tyrosine, serine, or threonine.

As defined above, R^{x} is a natural or unnatural amino acid side-chain group capable of forming cross-links. It will be appreciated that a variety of amino acid side-chain functional groups are capable of such cross-linking, including, but not limited to, carboxylate, hydroxyl, thiol, and amino groups. Examples of R^{x} moieties having functional groups capable of forming cross-links include a glutamic acid side-chain, —CH₂C(O)CH, an aspartic acid side-chain, —CH₂CH₂C(O)OH, a cystein side-chain, —CH₂SH, a serine side-chain, —CH₂OH, an aldehyde containing side-chain, —CH₂C(O)H, a lysine side-chain, —(CH₂)₄NH₂, an arginine side-chain, —(CH₂)₃NHC(═NH)NH₂, a histidine side-chain, —CH₂-imidazol-4-yl.

As used herein, the term “D,L-mixed poly(amino acid) block” refers to a poly(amino acid) block wherein the poly(amino acid) consists of a mixture of amino acids in both the D- and L-configurations. In certain embodiments, the D,L-mixed poly(amino acid) block is hydrophobic. In other embodiments, the D,L-mixed poly(amino acid) block consists of a mixture of D-configured hydrophobic amino acids and L-configured hydrophilic amino acid side-chain groups such that the overall poly(amino acid) block comprising is hydrophobic.

Thus, in certain embodiments, the R^{y} group of either of formula II or III forms a hydrophobic D,L-mixed poly(amino acid) block. Hydrophobic amino acid side-chain groups are well known in the art and include those described herein. In other embodiments, R^{y} consists of a mixture of D-hydrophobic and L-hydrophilic amino acid side-chain groups such that the overall poly(amino acid) block comprising R^{y} is hydrophobic and is a mixture of D- and L-configured amino acids. Such mixtures of amino acid side-chain groups include D-leucine/L-tyrosine, D-leucine/L-aspartic acid, D-leucine/L-glutamic acid, D-phenylalanine/L-tyrosine, D-phenylalanine/L-aspartic acid, D-phenylalanine/L-glutamic acid, D-phenylalanine/L-serine, D-benzyl aspartate/L-tyrosine, D-benzyl aspartate/L-aspartic acid, D-benzyl aspartate/L-glutamic acid, D-benzyl glutamate/L-tyrosine, D-benzyl glutamate/L-aspartic acid and the like. According to another embodiment, Ry is a hydrophobic amino acid side-chain group selected from D-leucine, D-phenylalanine, D-alanine, D-benzyl aspartate, or D-benzyl glutamate, and one or more of L-tyrosine, L-cysteine, L-aspartic acid, L-glutamic acid, L-DOPA, L-histidine, L-lysine, L-ornithine, or L-arginine.

In other embodiments, the R^{y} group of either of formula II or III consists of a mixture of D-hydrophobic and L-hydrophilic amino acid side-chain groups such that the overall poly(amino acid) block comprising R^{y} is hydrophobic and is a mixture of D- and L-configured amino acids. Such mixtures of amino acid side-chain groups include L-tyrosine and D-leucine, L-tyrosine and D-phenylalanine, L-serine and D-phenylalanine, L-aspartic acid and D-phenylalanine, L-glutamic acid and D-phenylalanine, L-tyrosine and D-benzyl glutamate, L-tyrosine and D-benzyl aspartate, L-serine and D-benzyl glutamate, L-serine and D-benzyl aspartate, L-aspartic acid and D-benzyl glutamate, L-aspartic acid and D-benzyl aspartate, L-glutamic acid and D-benzyl glutamate, L-glutamic acid and D-benzyl aspartate, L-aspartic acid and D-leucine, and L-glutamic acid and D-leucine. Ratios (D-hydrophobic to L-hydrophilic) of such amino acid combinations can range between 5-95 mol %.

One of ordinary skill in the art will appreciate that a compound of formula II is readily transformed into a compound of formula III using methods well known in the art. For example, the DFA salt of formula II may be treated with a suitable base to form a freebase compound. One of ordinary skill in the art would appreciate that a variety of bases are suitable for forming the free-base compound from the salt form of formula II. Such bases are well known in the art. In certain embodiments, the base utilized at step (d) is pyridine, or a derivative thereof, such as dimethylaminopyridine (“DMAP”), lutidine or collidine. In other embodiments, the base utilized at step (d) is dimethylaminopyridine (“DMAP”). In still other embodiments, inorganic bases are utilized and include ammonia, potassium hydroxide, sodium hydroxide, sodium carbonate, sodium bicarbonate, potassium carbonate, or potassium bicarbonate. Such a freebase compound may be further derivatized by treatment of that compound with a suitable terminating agent thereby introducing the R^{2a} moiety.

As described above, compounds of formula III are prepared from compounds of formula II by treatment with a base then a suitable terminating agent. One of ordinary skill in the art would recognize that compounds of formula III are also readily prepared directly from compounds of formula II. In such cases, and in certain embodiments, the compound of formula II is treated with a base to form the freebase compound prior to, or concurrent with, treatment with the suitable terminating agent. For example, it is contemplated that a compound of formula II is treated with a base and suitable terminating agent in the same reaction to form a compound of formula III. In such cases, it is also contemplated that the base may also serve as the reaction medium.

One of ordinary skill in the art would also recognize that the above method for preparing a compound of formula III may be performed as a “one-pot” synthesis of compounds of formula III that utilizes the living polymer chain-end to incorporate the R^{2a} group of formula III. Alternatively, compounds of formula III may also be prepared in a multi-step fashion. For example, the living polymer chain-end of a compound of formula II may be quenched to afford an amino group that may then be further derivatized, according to known methods, to afford a compound of formula III.

One of ordinary skill in the art will recognize that a variety of polymerization terminating agents are suitable for the present invention. Such polymerization terminating agents include any R^{2a}-containing group capable of reacting with the living polymer chain-end of a compound of formula II, or the free-based amino group of formula II, to afford a compound of formula III. Thus, polymerization terminating agents include anhydrides, and other acylating agents, and groups that contain a suitable leaving group L that is subject to nucleophilic displacement.

Alternatively, compounds of formula II, or freebase thereof, may be coupled to carboxylic acid-containing groups to form an amide thereof. Thus, it is contemplated that the amine group of formula II, or freebase thereof, may be coupled with a carboxylic acid moiety to afford compounds of formula III wherein R^{2a} is —NHC(O)R⁴. Such coupling reactions are well known in the art. In certain embodiments, the coupling is achieved with a suitable coupling reagent. Such reagents are well known in the art and include, for example, DCC and EDC, among others. In other embodiments, the carboxylic acid moiety is activated for use in the coupling reaction. Such activation includes formation of an acyl halide, use of a Mukaiyama reagent, and the like. These methods, and others, are known to one of ordinary skill in the art, e.g., see, “Advanced Organic Chemistry,” Jerry March, 5^{th} Ed., pp. 351-357, John Wiley and Sons, N.Y.

A “suitable leaving group that is subject to nucleophilic displacement” is a chemical group that is readily displaced by a desired incoming chemical moiety. Suitable leaving groups are well known in the art, e.g., see, March. Such leaving groups include, but are not limited to, halogen, alkoxy, sulphonyloxy, optionally substituted alkylsulphonyloxy, optionally substituted alkenylsulfonyloxy, optionally substituted arylsulfonyloxy, and diazonium moieties. Examples of suitable leaving groups include chloro, iodo, bromo, fluoro, methanesulfonyloxy (mesyloxy), tosyloxy, triflyloxy, nitro-phenylsulfonyloxy (nosyloxy), and bromo-phenylsulfonyloxy (brosyloxy).

According to an alternate embodiment, the suitable leaving group may be generated in situ within the reaction medium. For example, a leaving group may be generated in situ from a precursor of that compound wherein said precursor contains a group readily replaced by said leaving group in situ.

Alternatively, when the R^{2a} group of formula III is a mono- or di-protected amine, the protecting group(s) is removed and that functional group may be derivatized or protected with a different protecting group. It will be appreciated that the removal of any protecting group of the R^{2a} group of formula III is performed by methods suitable for that protecting group. Such methods are described in detail in Green.

In other embodiments, the R^{2a} group of formula III is incorporated by derivatization of the amino group of formula II, or freebase thereof, via anhydride coupling, optionally in the presence of base as appropriate. One of ordinary skill in the art would recognize that anhydride polymerization terminating agents containing an azide, an aldehyde, a hydroxyl, an alkyne, and other groups, or protected forms thereof, may be used to incorporate said azide, said aldehyde, said protected hydroxyl, said alkyne, and other groups into the R^{2a} group of compounds of formula III. It will also be appreciated that such anhydride polymerization terminating agents are also suitable for terminating the living polymer chain-end of a compound of formula II.

Another aspect of the present invention provides a method for preparing a multi-block copolymer of formula IV:

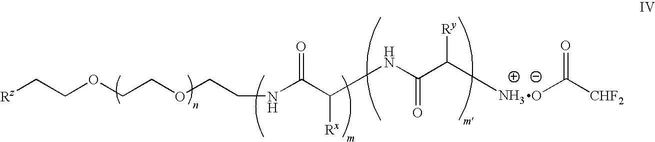

wherein:
n is 10-2500;
m is 0 to 1000;
m′ is 1 to 1000;
R^{x} is a natural or unnatural amino acid side-chain group that is capable of crosslinking;
R^{y} is a hydrophobic D,L-mixed amino acid side-chain group; and
R^{z} is CH₃O—, CH≡CCH₂O—, or N₃;
   wherein said method comprises the steps of:
(a) providing a compound of formula I-a:

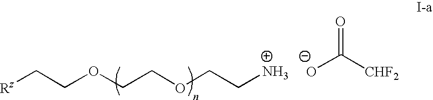

wherein:
R^{z} is CH₃O—, CH≡CCH₂O—, or N₃; and
n is 10-2500;
b) optionally polymerizing a first cyclic amino acid monomer onto the amine salt terminal end of formula I, wherein said first cyclic amino acid monomer comprises R^{x}; and
(c) polymerizing a second cyclic amino acid monomer, comprising R^{y}, onto the living polymer end, wherein said second cyclic amino acid monomer is different from said first cyclic amino acid monomer.

In some embodiments, the method for preparing a compound of formula IV further comprises the step of treating the compound of formula IV with a terminating agent to form a compound of formula V:

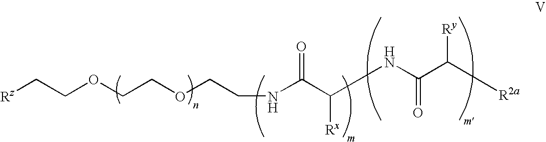

wherein each of R^{z}, n, R^{x}, m, R^{y}, m′, and R^{2a} are as defined above and described herein.

### EXAMPLES

As depicted in the Examples below, in certain exemplary embodiments, compounds are prepared according to the following general procedures. It will be appreciated that, although the general methods depict the synthesis of certain compounds of the present invention, the following general methods, in addition to the Schemes set forth above and other methods known to one of ordinary skill in the art, can be applied to all compounds and subclasses and species of each of these compounds, as described herein.

### Example 1

### Synthesis of Dibenzyl Amino Ethanol

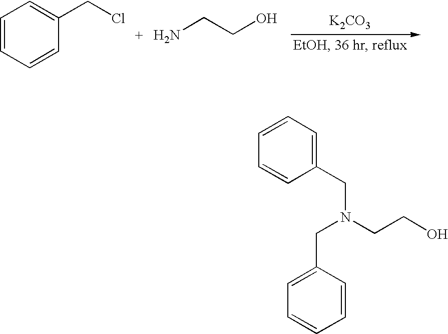

Benzyl chloride (278.5 g, 2.2 mol), ethanol amine (60 mL, 1 mol), potassium carbonate (283.1 g, 2.05 mol) and ethanol (2 L) were mixed together in a 3 L 3-neck flask, fitted with an overhead stirrer, a condenser and a glass plug. The whole setup was heated up to reflux for 36 hr, after which the insoluble solid was filtered through a medium frit. The filtrate was recovered and ethanol was concentrated in vacuo. The viscous liquid was re-dissolved in ether, the solid suspension removed by filtration and extracted twice against water. The ether solution was kept and the aqueous layer was extracted twice with dichloromethane (2×400 mL). The fraction were recombined, dried over MgSO₄, stirred over carbon black for 15 min and filtered through a Celite® pad. Dichloromethane was removed and the solid was re-dissolved into a minimal amount of ether (combined volume of 300 mL with the first ether fraction, 300 mL). Hexanes (1700 mL) was added and the solution was heated up gently till complete dissolution of the product. The solution was then cooled down gently, placed in the fridge (+4° C.) overnight and white crystals were obtained. The recrystallization was done a second time. 166.63 g, 69% yield. ¹H NMR (d₆-DMSO) δ 7.39-7.24 (10H), 4.42 (1H), 3.60 (4H), 3.52 (2H), 2.52 (2H).

### Example 2

### Synthesis of (Dibenzyl)-N-PEG₂₇₀-OH

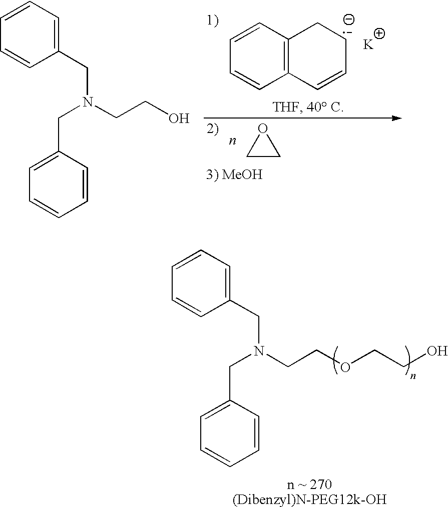

The glassware was assembled while still warm. Vacuum was then applied to the assembly and the ethylene oxide line to about 10 mTorr. The setup was backfilled with argon. 2-dibenzylamino ethanol (3.741 g, 40.4 mmol) was introduced via the sidearm of the jacketed flask under argon overpressure. Two vacuum/argon backfill cycles were applied to the whole setup. THF line was connected to the 14/20 side-arm and vacuum was applied to the whole setup. At this stage, the addition funnel was closed and left under vacuum. THF (4 L) was introduced via the side-arm in the round bottom flask under an argon overpressure. An aliquot of the THF added to the reaction vessel was collected and analyzed by Karl-Fisher colorometric titration to ensure water content of the THF is less than 6 ppm. Next, 2-dibenzylamino ethanol was converted to potassium 2-dibenzylamino ethoxide via addition of potassium naphthalenide (200 mL). Ethylene oxide (500 ml, 10.44 mol) was condensed under vacuum at −30° C. into the jacketed addition funnel, while the alkoxide solution was cooled to 10° C. Once the appropriate amount of ethylene oxide was condensed, the flow of ethylene oxide was stopped, and the liquid ethylene oxide added directly to the cooled alkoxide solution. After complete ethylene oxide addition, the addition funnel was closed and the reaction flask backfilled with argon. While stirring, the following temperature ramp was applied to the reaction: 12 hrs at 20° C., 1 hr from 20° C. to 40° C. and 3 days at 40° C. The reaction went from a light green tint to a golden yellow color. Upon termination with an excess methanol, the solution color changed to light green. The solution was precipitated into ether and isolated by filtration. 459 g, 99% yield was recovered after drying in a vacuum oven overnight. ¹H NMR (d6-DMSO) δ 7.4-7.2 (10H), 4.55 (1H), 3.83-3.21 (910H) ppm. PDI (DMF GPC)=1.03, Mₙ(MALDI-TOF)=11,560 g/mol

### Example 3

### Synthesis of H₂N-PEG₂₇₀-OH

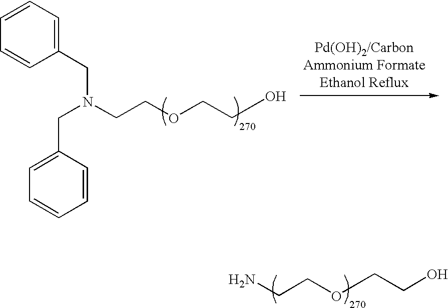

Batch Bz-EO270-OH-A (455 g, 39.56 mmol) was split into two equal amounts and was introduced into two 2 L flasks. Batch Bz-PEG₂₇₀-OH-B (273 g, 23.74 mmol) was put into a 2 L flask as well. The following steps were repeated for each flask. H₂N-EO270-OH (˜225 g), Pd(OH)₂/C (32 g, 45.6 mmol), ammonium formate (80 g, 1.27 mol) and ethanol (1.2 L) were mixed together in a 2 L flask. The reaction was heated to 80° C. while stirring for 24 hrs. The reaction was cooled to room temperature and filtered through a triple layer Celite®/MgSO₄/Celite® pad. The MgSO₄ powder is fine enough that very little Pd(OH)₂/C permeates through the pad. Celite® helps prevent the MgSO₄ layer from cracking. At this stage, the three filtrates were combined, precipitated into ˜30 L of ether and filtered through a medium glass frit. The wet polymer was then dissolved into 4 L of water, 1 L of brine and 400 mL of saturated K₂CO₃ solution. The pH was checked to be ˜11 by pH paper. The aqueous solution was introduced into a 12 L extraction funnel, rinsed once with 4 L of ether and extracted 4 times with dichloromethane (6 L, 6 L, 6 L, 2 L). Dichloromethane fractions were recombined, dried over MgSO₄ (3 kg), filtered, concentrated to ˜3 L by rotary evaporation and precipitated into diethyl ether (30 L). 555 g, 75% yield was recovered after filtration and evaporation to dryness in a vacuum oven. ¹H NMR (d6-DMSO) 4.55 (1H), 3.83-3.21 (910H), 2.96 (2H) ppm.

### Example 4

### Synthesis of H₂N-PEG₂₇₀-OH

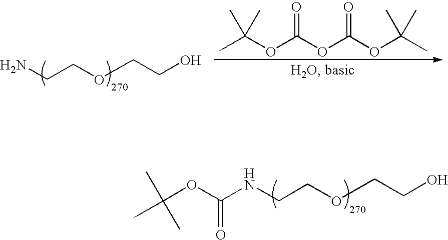

H₂N-PEG₂₇₀-OH (555 g, 48.26 mmol) was dissolved into 4 L of DI water. A saturated solution of K₂CO₃ (120 mL) was added, to keep the pH basic (pH ˜11 with pH paper). Di-tert-butyl dicarbonate (105 g, 0.48 mol) was added to the aqueous solution of H₂N-EO270-OH and allowed to stir at room temperature overnight. At this stage, a 5 mL aliquot of the reaction was extracted with 10 mL of dichloromethane and the dichloromethane extract precipitated into ether. A ¹H NMR was run to ensure completion of the reaction. Thereafter, the aqueous solution was placed into a 12 L extraction funnel, was rinsed once with ether (4 L) and extracted three times with dichloromethane (6 L, 6 L and 6 L). The organic fractions were recombined, dried over MgSO₄ (3 kg), filtered, concentrated to ˜4 L and precipitated into 30 L of ether. The white powder was filtered and dried overnight in a vacuum oven, giving 539 g, 97% yield. ¹H NMR (d6-DMSO) δ 6.75 (1H), 4.55 (1H), 3.83-3.21 (910H), 3.06 (2H), 1.37 (9H) ppm

### Example 5

### Reaction of Boc-HN-PEG₂₇₀-OH with Methanesulfonyl Chloride and Sodium Azide to Obtain Boc-HN-PEG₂₇₀-N₃

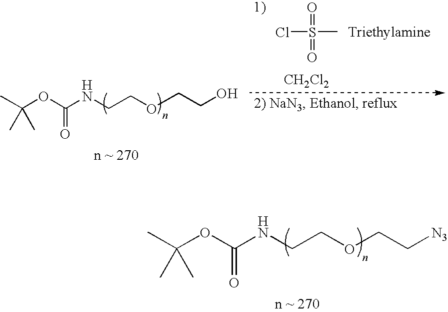

Boc-PEG₂₇₀-OH (539 g, 49.9 mmol) were placed into a 6 L jacketed flask and dried by azeotropic distillation from toluene (3 L). It was then dissolved into 3 L of dry dichloromethane under inert atmosphere. The solution was cooled to 0° C., methanesulfonyl chloride (10.9 mL, 140.8 mmol) was added followed by triethylamine (13.1 mL, 94 mmol). The reaction was allowed to warm to room temperature and proceeded overnight under inert atmosphere. The solution was evaporated to dryness by rotary evaporation and used as-is for the next step.

NaN₃ (30.5 g, 470 mmol) and 3 L of ethanol were added to the flask containing the polymer. The solution was heated to 80° C. and allowed to react overnight. It was then evaporated to dryness by rotary evaporation (bath temperature of 55° C.) and dissolved in 2 L of dichloromethane. The latter solution was the filtered through a Büchner funnel fitted with a Whatman paper #1 to remove most of the salts. The solution was concentrated down to ˜1 L by rotary evaporation. The product was purified by silica gel flash column chromatography using a 8 in. diameter column with a coarse frit. About 7 L of dry silica gel were used. The column was packed with 1:99 MeOH/CH₂Cl₂ and the product was loaded and eluted onto the column by pulling vacuum from the bottom of the column. The elution profile was the following: 1:99 MeOH/CH₂Cl₂ for 1 column volume (CV), 3:97 MeOH/CH₂Cl₂ for 2 CV and 10:90 MeOH/CH₂Cl₂ for 6 CV. The different polymer-containing fractions were recombined (˜40 L of dichloromethane), concentrated by rotary evaporation and precipitated into a 10-fold excess of diethyl ether. The polymer was recovered by filtration as a white powder and dried overnight in vacuo, giving 446.4 g, 82% yield. ¹H NMR (d₆-DMSO) δ 6.75 (1H), 3.83-3.21 (910H), 3.06 (2H), 1.37 (9H) ppm. Mₙ (MALDI-TOF)=11,554 g/mol. PDI (DMF GPC)=1.04

### Example 6

### Synthesis of N₃-PEG₂₇₀-NH₂/TFA Salt

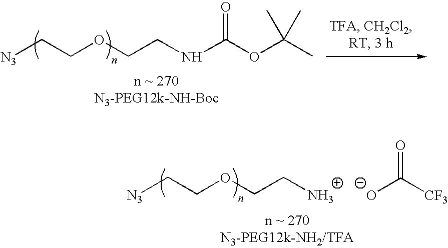

N₃-PEG₂₇₀-NH-Boc (10 g, 0.83 mmol) was dissolved in 50 mL of a TFA/CH₂Cl₂ (50/50 v/v) solution and stirred for 3 hours. The solution was then precipitated into a 10-fold excess of diethyl ether. After filtration, the white powder was dissolved in dichloromethane (50 mL) and precipitated again into diethyl ether. N₃-PEG₂₇₀-NH₃, TFA salt was recovered by filtration as a white powder and 9.09 g (yield=91%) were recovered after drying overnight in vacuo. ¹H NMR (d₆-DMSO) δ 7.67 (3H), 3.82-3.00 (1080H), 2.99 (2H).

### Example 7

### Synthesis of N₃-PEG12K-NH₂/DFA Salt

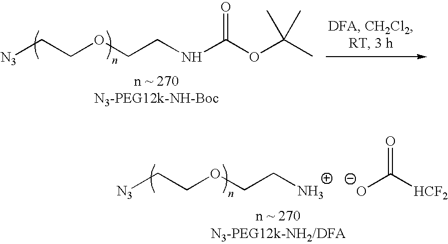

N₃-PEG12K-NH-Boc (10 g, 0.83 mmol) was dissolved in a 25 mL:16.2 mL mixture of a DFA/CH₂Cl₂ solution and stirred for 3 hours. The solution was then precipitated into a 10-fold excess of diethyl ether. After filtration, the white powder was dissolved in dichloromethane (50 mL) and precipitated again into diethyl ether. N₃-PEG12K-NH₃, TFA salt was recovered by filtration as a white powder and 8.96 g (yield=90%) were recovered after drying overnight in vacuo. ¹H NMR (d₆-DMSO) δ 7.67 (3H), 6.13 (1H), 3.82-3.00 (1080H), 2.99 (2H).

### Example 8

### Synthesis of N₃-PEG12K-NH₂/DCA Salt

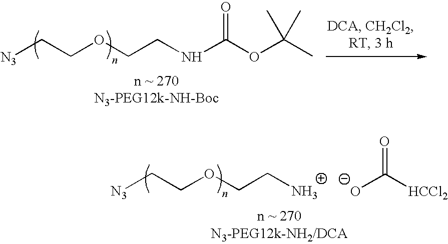

N₃-PEG12K-NH-Boc (10 g, 0.83 mmol) was dissolved in a 10 mL:40 mL mixture of a DCA/CH₂Cl₂ solution and stirred for 3 hours. The solution was then precipitated into a 10-fold excess of diethyl ether. After filtration, the white powder was dissolved in dichloromethane (50 mL) and precipitated again into diethyl ether. N₃-PEG12K-NH₃, TFA salt was recovered by filtration as a white powder and 9.05 g (yield=90%) were recovered after drying overnight in vacuo. ¹H NMR (d6-DMSO) δ 7.67 (3H), 6.49 (1H), 3.82-3.00 (1080H), 2.99 (2H).

### Example 9

### Synthesis of N₃-PEG10K-NH₃Cl Salt

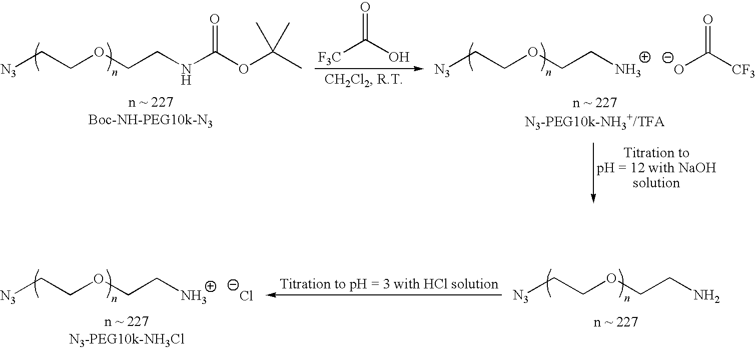

N₃-PEG10K-NH-Boc (52 g, 5.2 mmol) was dissolved in 400 mL of a TFA/CH₂Cl₂ (50/50 v/v) solution and stirred for 2 hours. The solution was then precipitated into a 10-fold excess of diethyl ether. After filtration, the white powder was dissolved in dichloromethane and precipitated again into diethyl ether. N₃-PEG 10K-NH₃, TFA salt was recovered by filtration as a white powder. The polymer was then dissolved into 200 mL of a brine/water (50/50 v/v) mixture and neutralized to pH 12 by drop wise addition of a 5N sodium hydroxide solution. The product was extracted three times with dichloromethane. The dichloromethane fractions were combined, dried over MgSO₄, filtered, concentrated on the rotary evaporator, and precipitated into an excess of diethyl ether. N₃-PEG10K-NH₂ was isolated by filtration as a white powder. The polymer was dissolved into 200 mL of a 50:50 brine/water (50/50 v/v) mixture and the pH was adjusted to 3 by drop wise addition of a 3N hydrochloric acid solution. The product was extracted three times with dichloromethane. The dichloromethane fractions were combined, dried over MgSO₄, filtered, concentrated on the rotary evaporator, and precipitated into an excess of diethyl ether. N₃-PEG10K-NH₃Cl was isolated by filtration and dried in vacuo to yield 48 g (92% yield) of a white powder. ¹H NMR (d₆-DMSO) 7.77 (3H), 3.83-3.21 (910H), 2.98 (2H) ppm.

### Example 10

### Synthesis of D-Leucine NCA

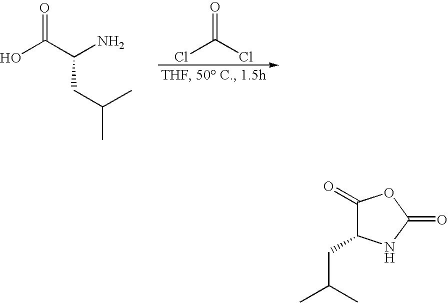

H-DLeu-OH (20.0 g, 152.5 mmol) was suspended in 300 mL of anhydrous THF and heated to 50° C. Phosgene (20% in toluene) (99.3 mL, 198.3 mmol) was added to the amino acid suspension. The amino acid dissolved over the course of approx. 1 hr, forming a clear solution. The solution was concentrated in vacuo, transferred to a beaker, and hexane was added to precipitate the product. The white solid was isolated by filtration and dissolved in a toluene/THF mixture. The solution was filtered over a bed of Celite® to remove any insoluble material. An excess of hexane was added to the filtrate to precipitate the product. The NCA was isolated by filtration and dried in vacuo. 13.8 g (58% yield) of DLeu NCA was isolated as a white, crystalline solid. ¹H NMR (d₆-DMSO) δ 9.13 (1H), 4.44 (1H), 1.74 (1H), 1.55 (2H), 0.90 (6H) ppm.

### Example 11

### Synthesis of Asp(O^{t}Bu)NCA

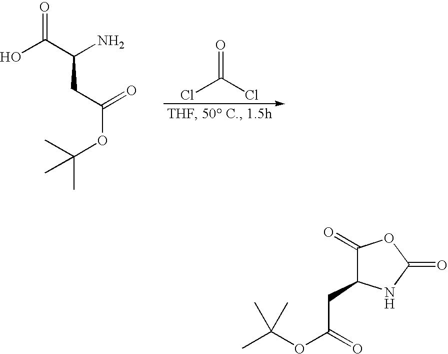

H-Asp(O^{t}Bu)—OH (25.0 g, 132 mmol) was suspended in 500 mL of anhydrous THF and heated to 50° C. Phosgene (20% in toluene) (100 mL, 200 mmol) was added to the amino acid suspension, and the amino acid dissolved over the course of approx. 1 hr, forming a clear solution. The solution was concentrated on by rotary evaporation, transferred to a beaker, and hexane was added to precipitate the product. The white solid was isolated by filtration and dissolved in anhydrous THF. The solution was filtered over a bed of Celite® to remove any insoluble material. An excess of hexane was added on the top of the filtrate and the bilayer solution was left in the freezer overnight. The NCA was isolated by filtration and dried in vacuo. 13.1 g (46% yield) of Asp(O^{t}Bu)NCA was isolated as a white, crystalline solid. ¹H NMR (d₆-DMSO) δ 8.99 (1H), 4.61 (1H), 2.93 (1H), 2.69 (1H), 1.38 (9H) ppm.

### Example 12

### Synthesis of Tyr(OBzl) NCA

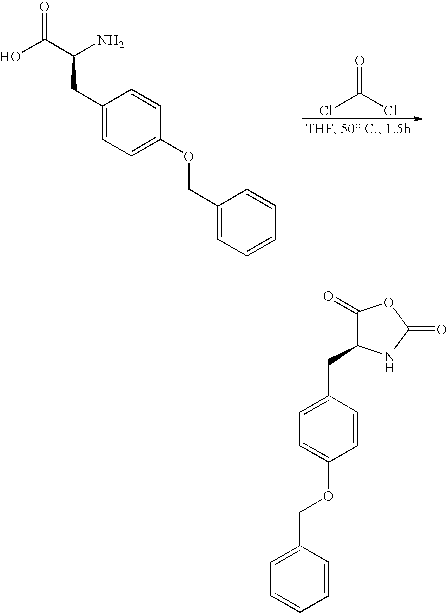

H-Tyr(OBzl)-OH (20.0 g, 105.7 mmol) was suspended in 300 mL of anhydrous THF and heated to 50° C. Phosgene (20% in toluene) (73.7 mL, 147.4 mmol) was added the amino acid suspension. The amino acid dissolved over the course of approx. 1 hr, forming a pale yellow solution. The solution was concentrated in vacuo, transferred to a beaker, and hexanes were added to precipitate the product. The NCA was isolated by filtration and dried in vacuo. 11.74 g (75% yield) of Asp(OBzl) NCA was isolated as a white solid. ¹H NMR (d₆-DMSO) δ 8.99 (1H), 7.42-7.18 (5H), 5.10 (2H), 4.65 (1H), 3.1-2.80 (2H) ppm.

### Example 13

### Synthesis of Asp(OBzl)NCA

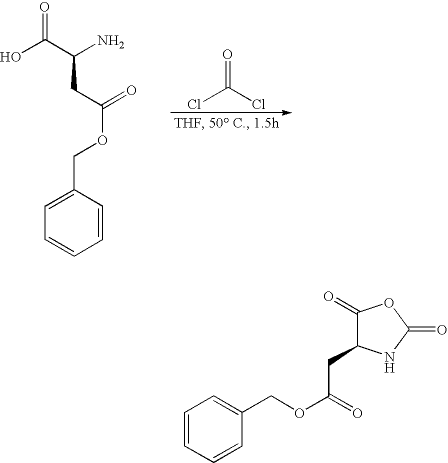

H-Asp(OBzl)-OH (14.0 g, 62.7 mmol) was suspended in 225 mL of anhydrous THF and heated to 50° C. Phosgene (20% in toluene) (40 mL, 80 mmol) was added the amino acid suspension. The amino acid dissolved to give a clear solution over the course of approx. 15 min and was left reacting for another 25 min. The solution was concentrated in vacuo, the white solid re-dissolved in a toluene/THF mixture (100 mL/50 mL) and the clear solution concentrated in vacuo to dryness. The white solid obtained was re-dissolved into 100 mL of THF, transferred to a beaker, and dry hexanes were added to precipitate the product. The white solid was isolated by filtration and rinsed twice with dry hexanes (2×200 mL) The NCA was isolated by filtration and dried in vacuo. 14.3 g (65% yield) of Asp(OBzl) NCA was isolated as a white solid. ¹H NMR (d₆-DMSO) δ 9.00 (1H), 7.48-7.25 (5H), 5.13 (2H), 4.69 (1H), 3.09 (1H), 2.92 (1H) ppm

### Example 14

### Synthesis of D-Asp(OBzl)NCA

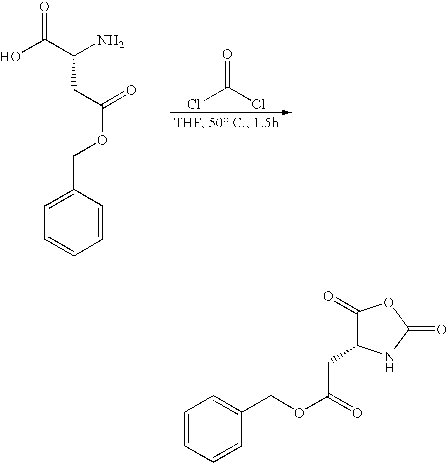

H-D-Asp(OBzl)-OH (30.0 g, 134 mmol) was suspended in 450 mL of anhydrous THF and heated to 50° C. Phosgene (20% in toluene) (100 mL, 100 mmol) was added the amino acid suspension. The amino acid dissolved over the course of approx. 50 min and was left reacting for another 30 min. The solution was concentrated in vacuo, the white solid re-dissolved in a toluene/THF mixture (250 mL/50 mL) and the clear solution concentrated in vacuo to dryness. The white solid obtained was re-dissolved into 250 mL of THF, transferred to a beaker, and dry hexanes were added to precipitate the product. The white solid was isolated by filtration and rinsed twice with dry hexanes (2×400 mL). The NCA was isolated by filtration and dried in vacuo. 26.85 g (83.2% yield) of D-Asp(OBzl) NCA was isolated as a white solid. ¹H NMR (d₆-DMSO) δ 9.00 (1H), 7.48-7.25 (5H), 5.13 (2H), 4.69 (1H), 3.09 (1H), 2.92 (1H) ppm

### Example 15

### Synthesis of D-PheNCA

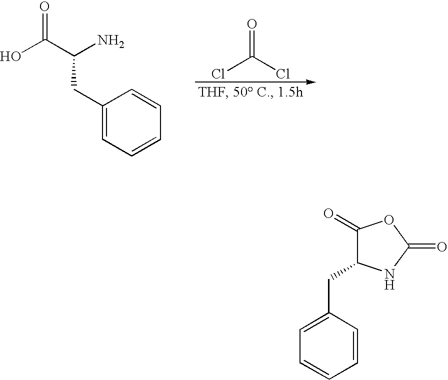

H-D-Phe-OH (20.0 g, 132 mmol) was suspended in 300 mL of anhydrous THF and heated to 50° C. Phosgene (20% in toluene) (90 mL, 182 mmol) was added to the amino acid suspension, and the amino acid dissolved over the course of approx. 1 hr, forming a cloudy solution. The solution was filtered through a paper filter (Whatman #1), concentrated on by rotary evaporation, transferred to a beaker, and hexane was added to precipitate the product. The white solid was isolated by filtration and dissolved in anhydrous THF. The solution was filtered over a bed of Celite® to remove any insoluble material. An excess of hexanes were added on the filtrate while stirring with a spatula. The NCA was isolated by filtration and dried in vacuo. 20.0 g (86% yield) of D-PheNCA was isolated as a white, crystalline solid. ¹H NMR (d₆-DMSO) δ 9.09 (1H), 7.40-7.08 (5H), 4.788 (1H), 3.036 (2H) ppm.

### Example 16

### Synthesis of Orn(Z)NCA

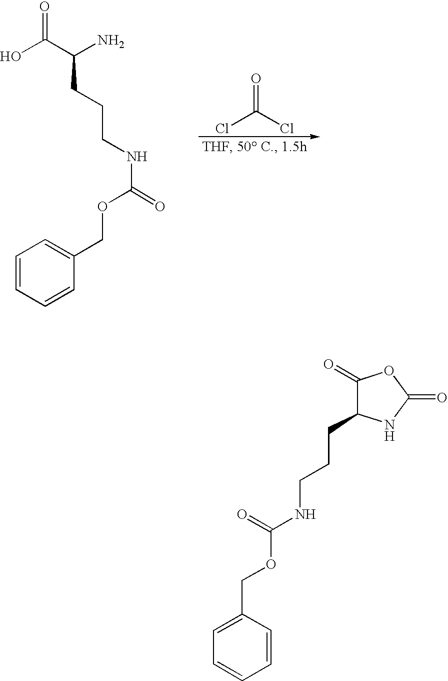

H-Orn(Z)-OH (35.4 g, 133 mmol) was suspended in 525 mL of anhydrous THF and heated to 50° C. Phosgene (20% in toluene) (100 mL, 200 mmol) was added to the amino acid suspension, and the amino acid dissolved over the course of approx. 1.5 hr, forming a clear solution. The solution was filtered through a paper filter (Whatman #1), concentrated on by rotary evaporation, transferred to a beaker, and hexane was added to precipitate the product. The white solid was isolated by filtration and dissolved in anhydrous THF. The solution was filtered over a bed of Celite® to remove any insoluble material. An excess of hexanes were added on the filtrate while stirring with a spatula. The NCA was isolated by filtration and dried in vacuo. 34.7 g (89% yield) of Orn(Z) NCA was isolated as a white, crystalline solid. ¹H NMR (d₆-DMSO) δ 9.09 (1H), 7.48-7.25 (5H), 5.01 (2H), 4.44 (1H), 3.02 (2H), 1.80-1.69 (2H), 1.69-1.58 (2H), 1.56-1.38 (2H) ppm.

### Example 17

### Synthesis of N₃-PEG10K-b-Poly(Asp(^{t}OBu)₁₀)-b-Poly(D-Leu₂₀-co-Tyr(OBzl)₂₀)-Ac from TFA Salt

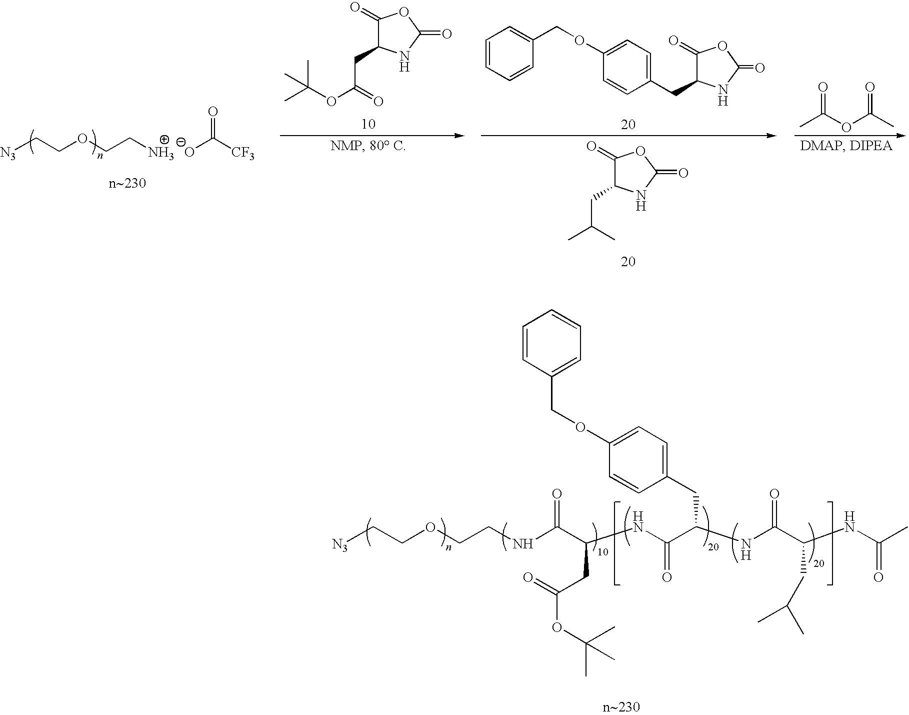

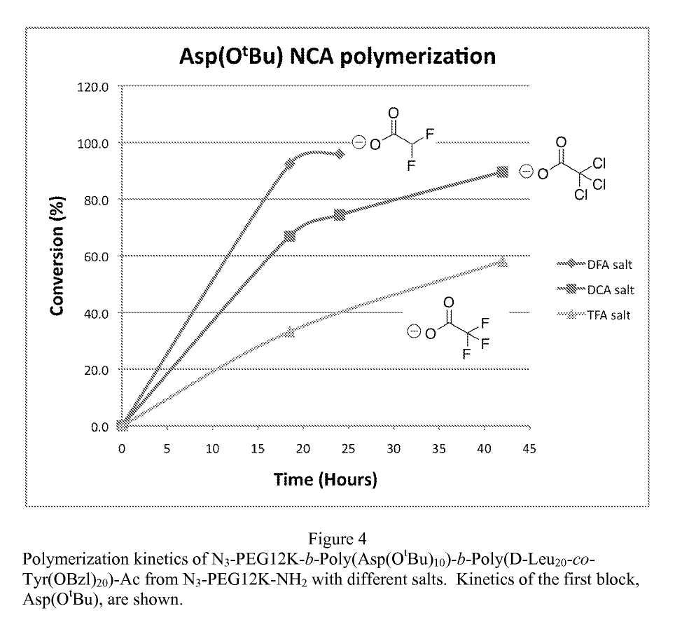

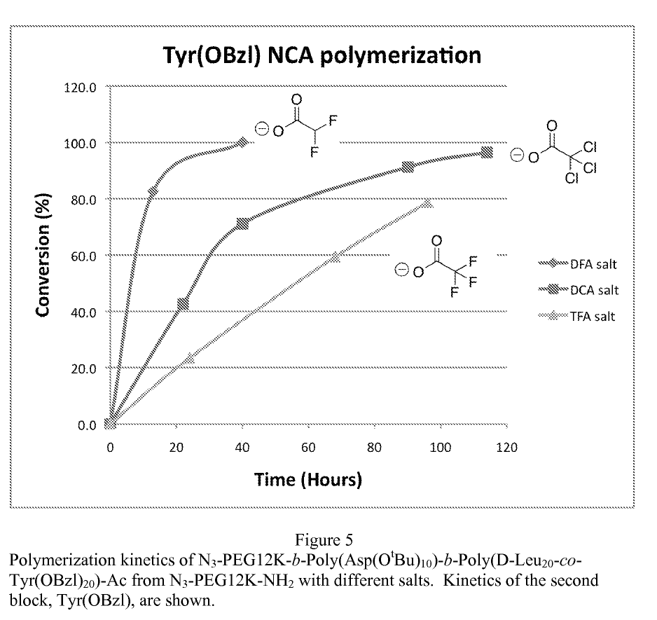

N₃-PEG10K-NH₂/TFA salt, (2.0 g, 0.2 mmol) was weighed into an oven-dried, round-bottom flask, dissolved in toluene, and dried by azeotropic distillation. Excess toluene was removed under vacuum. Asp(O^{t}Bu) NCA (0.43 g, 2.0 mmol) and pyrene (50 mg, 0.25 mmol) was added to the flask, the flask was evacuated under reduced pressure, and subsequently backfilled with nitrogen gas. Dry N-methylpyrrolidone (NMP) (12.1 mL) was introduced by syringe and the solution was heated to 80° C. The reaction mixture was allowed to stir for 24 hours at 80° C. under nitrogen gas. In an oven-dried round-bottom flask, D-Leu NCA (0.63 g, 4.0 mmol) and Tyr(OBzl) NCA (1.2 g, 4.0 mmol) were combined and dissolved in 9.1 ml of dry NMP under nitrogen gas. This solution was then transferred to the polymerization by syringe and allowed to stir for an additional 40 hours at 80° C. under nitrogen gas. Reaction kinetic was followed throughout the reaction. At different time points, 0.1 mL of the reaction solution was aliquoted, dried under vacuum and redissolved into 5 mL of acetonitrile. A fraction of the latter solution was injected in HPLC and conversion was calculated using pyrene as an internal standard. The results of the kinetic study are reported in FIG. 4 and FIG. 5. Numerical values for the kinetic study can be seen in the Table 1 below. The solution was cooled to room temperature and diisopropylethylamine (DIPEA) (1.0 mL), dimethylaminopyridine (DMAP) (100 mg), and acetic anhydride (1.0 mL) were added. Stirring was continued for 1 hour at room temperature. The polymer was precipitated into diethyl ether and isolated by filtration. The solid was redissolved in dichloromethane and precipitated into diethyl ether. The product was isolated by filtration and dried in vacuo to give the block copolymer as an off-white powder. ¹H NMR (d₆-DMSO) δ 12.2 (2H), 9.1 (13H), 8.51-7.71 (49H), 6.96 (29H), 6.59 (26H), 4.69-3.96 (59H), 3.81-3.25 (1040H), 3.06-2.65 (45H), 1.0-0.43 (139). ¹³C NMR (d₆-DMSO) δ 171.9, 171, 170.5, 170.3, 155.9, 130.6, 129.6, 127.9 115.3, 114.3, 70.7, 69.8, 54.5, 51.5, 50, 49.8, 49.4, 36.9, 36, 24.3, 23.3, 22.3, 21.2. IR (ATR) 3290, 2882, 1733, 1658, 1342, 1102, 962 cm⁻¹ PDI (DMF GPC)=1.04

TABLE 1
Kinetics of polymerization from TFA salt (Top: Second
block kinetics, Bottom: Third Block Kinetics).
Polymerization from N₃-PEG10K-NH₂/TFA
Second Block

| Time (h) | Asp(O^{t}Bu)NCA | Pyrene | Conversion |
|---|---|---|---|
| 0 | 3176018 | 7949044 | 0.0 |
| 18.5 | 1665492 | 6240689 | 33.2 |
| 42 | 1124096 | 6715988 | 58.1 |

Polymerization from N₃-PEG10K-NH₂/TFA
Third Block

| Time (h) | Tyr(OBzl)NCA | Pyrene | Conversion |
|---|---|---|---|
| 0 | 75204568 | 3582657 | 0.0 |
| 24 | 63204099 | 3930706 | 23.4 |
| 68 | 28056646 | 3284318 | 59.3 |
| 96 | 17448066 | 3894843 | 78.7 |

### Example 18

### Synthesis of N₃-PEG10K-b-Poly(Asp(O^{t}Bu)₁₀)-b-Poly(D-Leu₂₀-co-Tyr(OBzl)₂₀)-Ac from DFA Salt

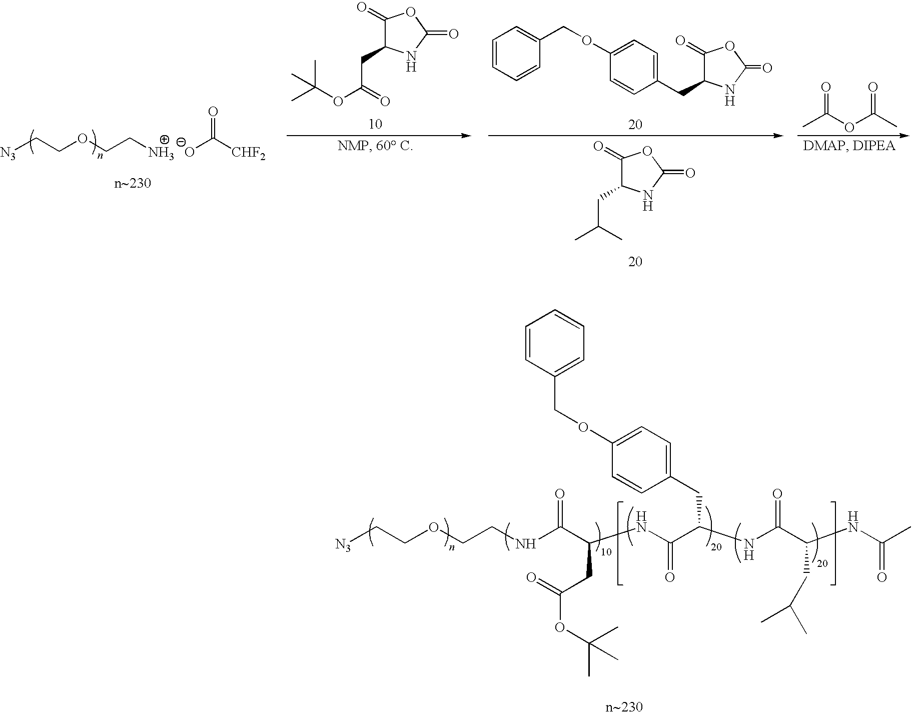

The same protocol as in Example 15 was used, starting with N₃-PEG10K-NH₂/DFA salt as an initiator. Plot of the kinetic study are reported in FIG. 4 and FIG. 5. The product was isolated by filtration and dried in vacuo to give 2.8 g (73% yield) of triblock copolymer as an off-white powder. Numerical values for the kinetic study can be seen in the Table 2 below. ¹H NMR (d₆-DMSO) δ 12.2 (2H), 9.1 (13H), 8.51-7.71 (49H), 6.96 (29H), 6.59 (26H), 4.69-3.96 (59H), 3.81-3.25 (1040H), 3.06-2.65 (45H), 1.0-0.43 (139). ¹³C NMR (d₆-DMSO) δ 171.9, 171, 170.5, 170.3, 155.9, 130.6, 129.6, 127.9 115.3, 114.3, 70.7, 69.8, 54.5, 51.5, 50, 49.8, 49.4, 36.9, 36, 24.3, 23.3, 22.3, 21.2. IR (ATR) 3290, 2882, 1733, 1658, 1342, 1102, 962 cm⁻¹. Mₙ (MALDI-TOF)=17,300 g/mol. PDI (DMF GPC)=1.05

TABLE 2
Kinetics of polymerization from DFA salt (Top: Second
block kinetics, Bottom: Third Block Kinetics).
Polymerization from N₃-PEG10K-NH₂/DFA
Second Block

| Time (h) | Asp(O^{t}Bu)NCA | Pyrene | Conversion |
|---|---|---|---|
| 0 | 2326164 | 6028039 | 0.0 |
| 18.5 | 205387 | 6919229 | 92.3 |
| 24 | 86782 | 5439847 | 95.9 |

Polymerization from N₃-PEG10K-NH₂/DFA
Third Block

| Time | Tyr(OBzl)NCA | Pyrene | Conversion |
|---|---|---|---|
| 0 | 44915015 | 2792211 | 0.0 |
| 13 | 10089934 | 3612647 | 82.6 |
| 40 | 0 | 5439847 | 100.0 |

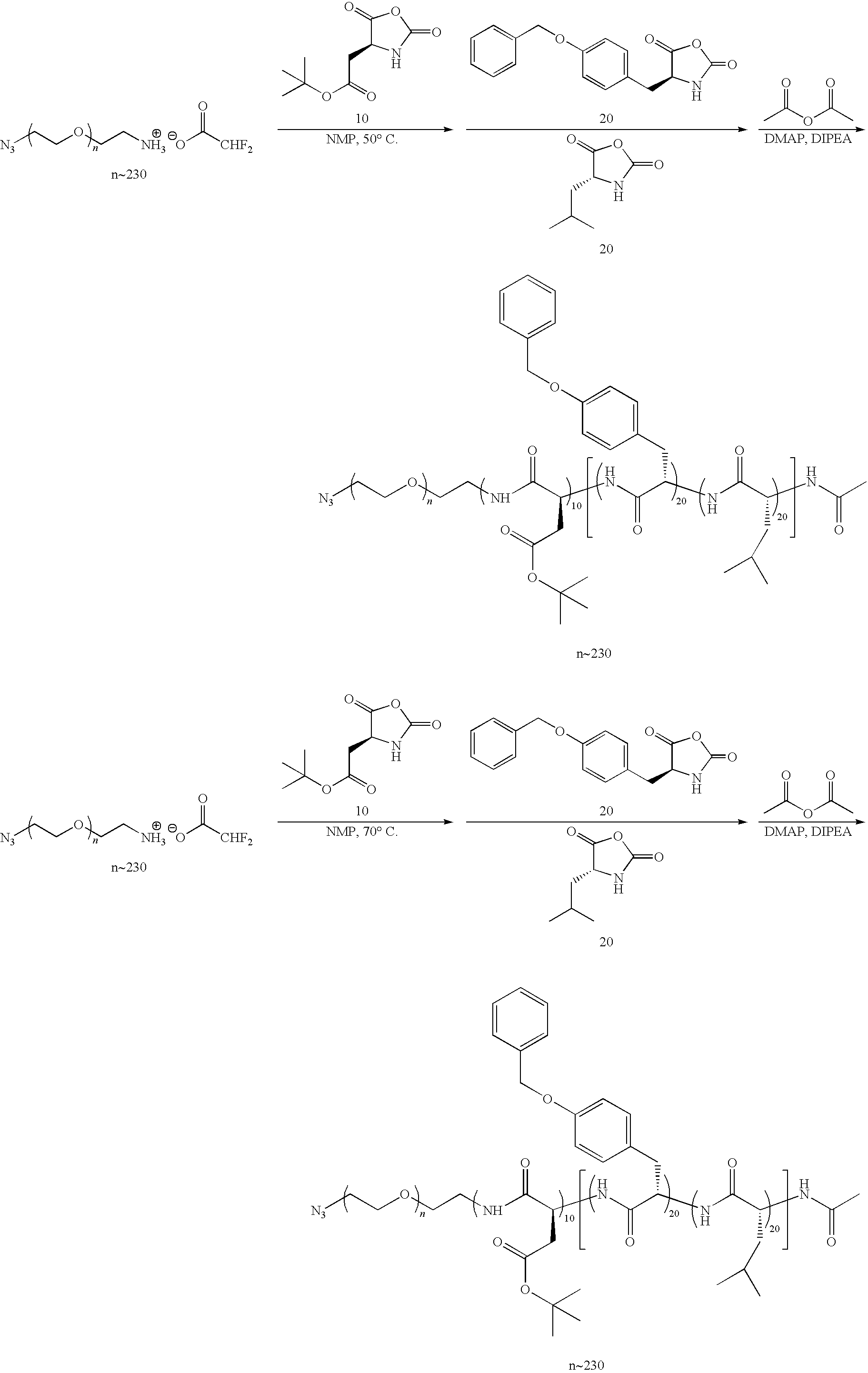

### Example 19

### Synthesis of N₃-PEG12K-b-Poly(Asp(^{t}OBu)₁₀)-b-Poly(d-Leu₂₀-co-Tyr(OBzl)₂₀)-Ac from DCA Salt

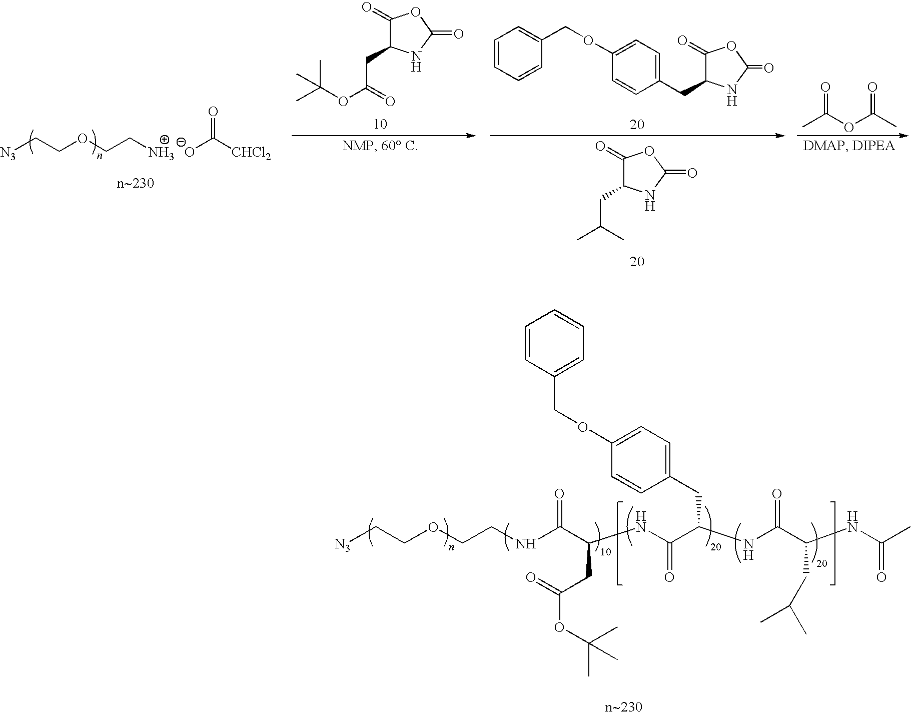

The same protocol as in Example 15 was used, starting with N₃-PEG10K-NH₂/DCA salt as an initiator. Results of the kinetic study are reported in FIG. 4 and FIG. 5. The product was isolated by filtration and dried in vacuo to give the triblock copolymer as an off-white powder. Characterizations were identical to Example 17. Numerical values for the kinetic study can be seen in the Table 3 below.

TABLE 3
Kinetics of polymerization from DCA salt (Top: Second
block kinetics, Bottom: Third Block Kinetics).
Polymerization from N₃-PEG10K-NH₂/DCA
Second Block

| Time (h) | Asp(O^{t}Bu)NCA | Pyrene | Conversion |
|---|---|---|---|
| 0 | 2777319 | 6360315 | 0.0 |
| 18.5 | 829773 | 5742086 | 66.9 |
| 24 | 828319 | 7399944 | 74.4 |
| 42 | 258660 | 5677385 | 89.6 |
| 114 | 2171325 | 3076442 | 96.5 |

Polymerization from N₃-PEG10K-NH₂/DCA
Third Block

| Time | Tyr(OBzl)NCA | Pyrene | Conversion |
|---|---|---|---|
| 0 | 66353508 | 3335796 | 0.0 |
| 22 | 51047367 | 4471412 | 42.6 |
| 40 | 22565863 | 3924696 | 71.1 |
| 90 | 6244553 | 3571676 | 91.2 |
| 114 | 2171325 | 3076442 | 96.5 |

### Example 20

### Synthesis of N₃-PEG10K-b-Poly(Asp(O^{t}Bu)₁₀)-b-Poly(D-Leu₂₀-co-Tyr(OBzl)₂₀)-Ac from HCl Salt

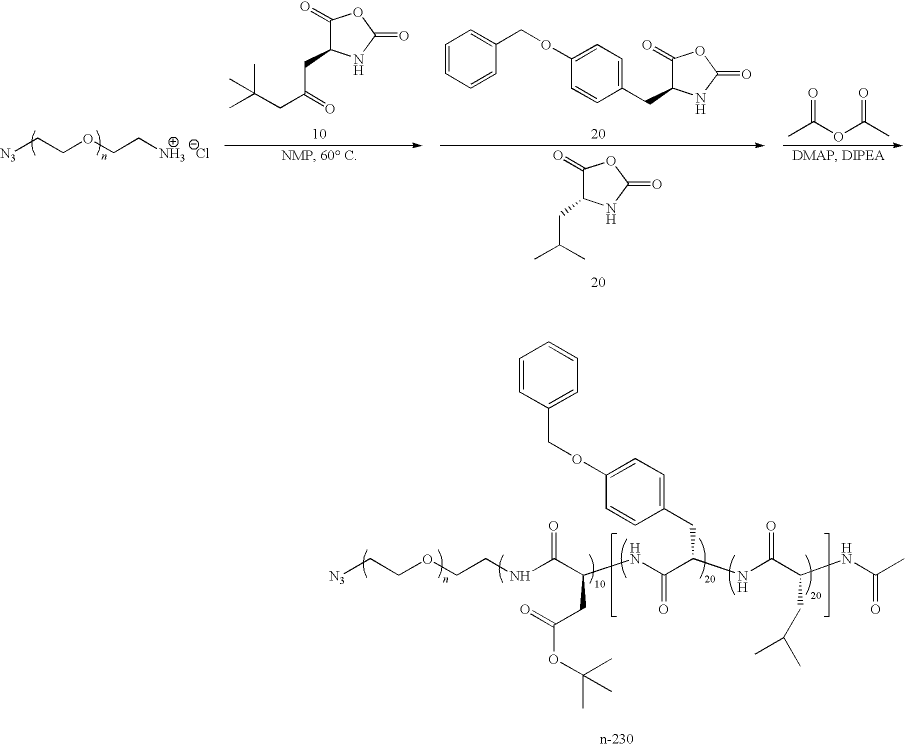

N₃-PEG10K-NH₃HCl salt, (10.0 g, 0.97 mmol) was weighed into an oven-dried, round-bottom flask, dissolved in toluene, and dried by azeotropic distillation. Excess toluene was removed under vacuum. Asp(O^{t}Bu) NCA (2.09 g, 9.7 mmol) was added to the flask, the flask was evacuated under reduced pressure, and subsequently backfilled with nitrogen gas. Dry N-methylpyrrolidone (NMP) (60 mL) was introduced by syringe and the solution was heated to 80° C. The reaction mixture was allowed to stir for 48 hours at 80° C. under nitrogen gas. In an oven-dried round-bottom flask, D-Leu NCA (3.04 g, 19.3 mmol) and Tyr(Bzl) NCA (5.77 g, 19.4 mmol) were combined and dissolved in 44.0 ml of dry NMP under nitrogen gas. This solution was then transferred to the polymerization by syringe and allowed to stir for an additional 120 hours at 80° C. under nitrogen gas. Reaction kinetic was followed throughout the reaction. At different time points, 0.1 mL of the reaction solution was aliquoted, dried under vacuum and re-dissolved into 5 mL of acetonitrile. A fraction of the latter solution was injected in HPLC and conversion was calculated using pyrene as an internal standard. A Waters HPLC (Model 2695) equipped with a Waters Photodiode Array Detector 996 was used. The mobile phase was a 50:50 mixture of acetonitrile: water. A Chromegabond Alkyl Phenyl (ES Industries Chromega Columns) was used as the stationary phase. Plots of the kinetic study are reported in FIG. 4. Kinetics results can be seen below in Table 1. The solution was cooled to room temperature and DIPEA (1.0 mL), DMAP (100 mg), and acetic anhydride (1.0 mL) were added. Stirring was continued for 1 hour at room temperature. The polymer was precipitated into diethyl ether and isolated by filtration. The solid was re-dissolved in dichloromethane and precipitated into diethyl ether. The product was isolated by filtration and dried in vacuo to give the block copolymer as an off-white powder. ¹H NMR (d₆-DMSO) δ 7.70-8.40, 7.35, 7.09, 6.82, 4.96, 4.50, 4.00-4.20, 3.20-3.7, 2.90, 2.70, 1.36, 0.40-0.90 ppm.

### Example 21

### Synthesis of N₃-PEG12K-b-Poly(Asp(O^{t}Bu)₁₀)-b-Poly(D-Leu₂₀-co-Tyr(OBzl)₂₀)-Ac from HCl Salt

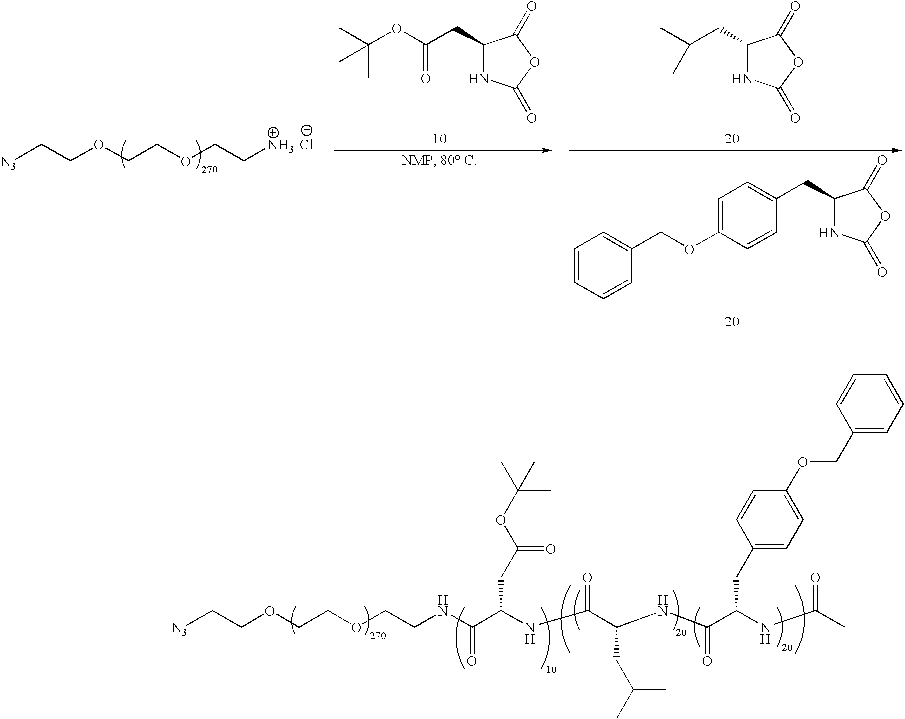

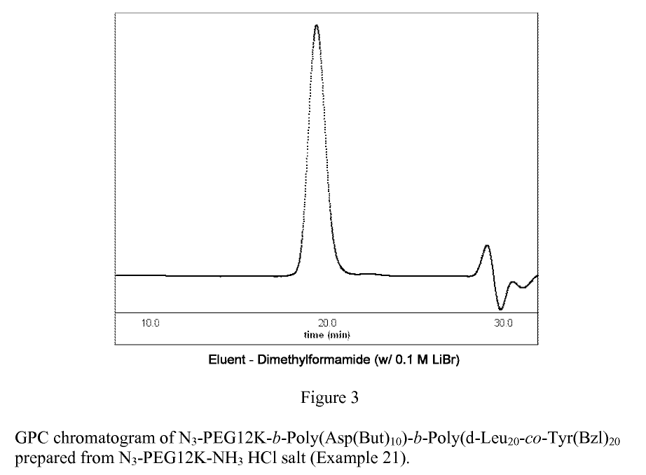

N₃-PEG 12K-b-Poly(Asp(O^{t}Bu)₁₀)-b-Poly(D-Leu₂₀-co-Tyr(OBzl)₂₀)-Ac was synthesized as described in Example 11 from N₃-PEG-NH₃HCl salt, 12 kDa (5.0 g, 0.42 mmol), Asp(But) NCA (0.9 g, 4.2 mmol), D-Leu NCA (0.9 g, 5.4 mmol), and Tyr(OBzl) NCA (2.1 g, 7.1 mmol). The block copolymer was isolated as an off-white powder. ¹H NMR (d₆-DMSO) δ 7.70-8.40, 7.35, 7.09, 6.82, 4.96, 4.50, 4.00-4.20, 3.20-3.7, 2.90, 2.70, 1.36, 0.40-0.90 ppm. A GPC trace of the final product can be seen in FIG. 3.

### Example 22

### Synthesis of N₃-PEG12K-b-P(Asp₁₀)-b-P(D-Leu₂₀-co-Tyr₂₀)-Ac

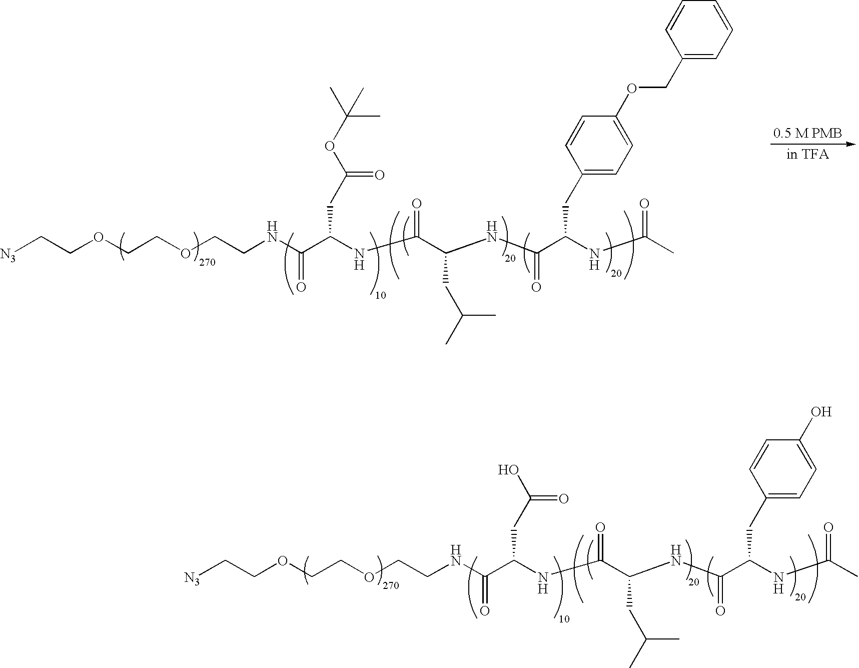

N₃-PEG12K-b-Poly(Asp(O^{t}Bu)₁₀)-b-Poly(DLeu₂₀-co-Tyr(OBzl)₂₀)-Ac (5.0 g, 0.22 mmol) was dissolved in 100 mL of a 0.5 M solution of pentamethylbenzene (PMB) in trifluoroacetic acid (TFA). The reaction was allowed to stir for 2.5 hours at room temperature with a white precipitate forming after approximately 1 hour. The solution was precipitated into a 10-fold excess of diethyl ether and the polymer was recovered by filtration. The polymer was dissolved into dichloromethane and re-precipitated into diethyl ether. The polymer was isolated by filtration and dried in vacuo to yield 3.1 g (60% yield) of an off-white powder. ¹H NMR (d₆-DMSO) δ 12.35, 9.10, 7.60-8.60, 6.96, 6.60, 4.50, 4.40, 4.10-4.25, 3.20-3.70, 2.85, 2.70, 0.40-1.40 ppm.

### Example 23

### Synthesis of N₃-PEG12K-b-P(Asp(O^{t}Bu)₁₀)-b-P(D-Leu₃₀-co-Asp(O^{t}Bu)₃₀)-Ac

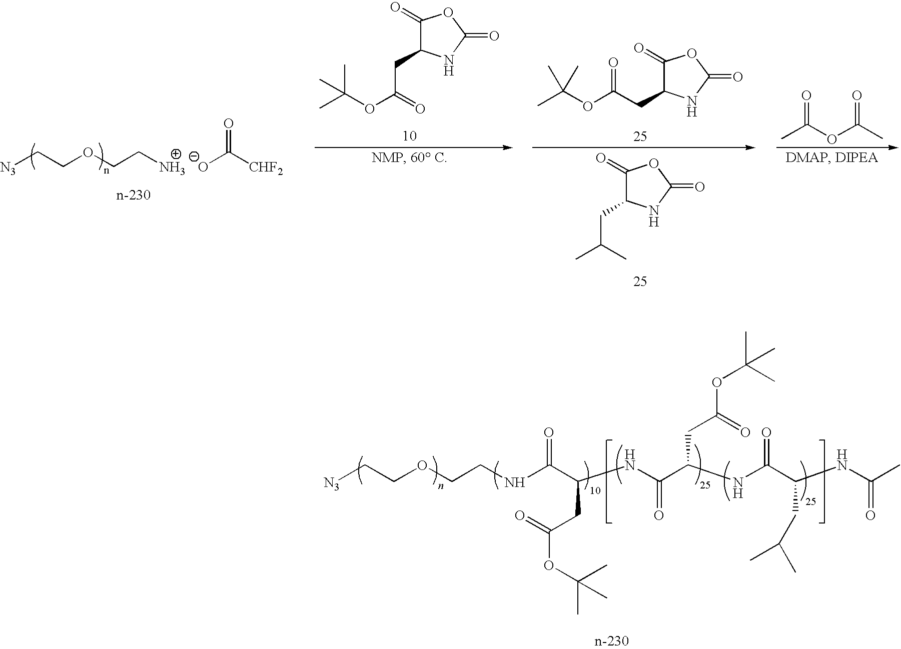

N₃-PEG10K-NH₂/DFA salt, (10.0 g, 1 mmol) was weighed into an oven-dried, round-bottom flask, dissolved in toluene, and dried by azeotropic distillation. Excess toluene was removed under vacuum. Asp(O^{t}Bu) NCA (2.15 g, 10 mmol) was added to the flask, the flask was evacuated under reduced pressure, and subsequently backfilled with nitrogen gas. Dry N-methylpyrrolidone (NMP) (60 mL) was introduced by syringe and the solution was heated to 60° C. The reaction mixture was allowed to stir for 15 hours at 80° C. under nitrogen gas. In an oven-dried round-bottom flask, D-Leu NCA (3.93 g, 25 mmol) and Asp(O^{t}Bu) NCA (5.38 g, 25 mmol) were combined and dissolved in 46.0 ml of dry NMP under nitrogen gas. This solution was then transferred to the polymerization by syringe and allowed to stir for an additional 24 hours at 60° C. The solution was cooled to room temperature and DIPEA (1.0 mL), DMAP (100 mg), and acetic anhydride (1.0 mL) were added. Stirring was continued for 1 hour at room temperature. The polymer was precipitated into diethyl ether and isolated by filtration. The solid was re-dissolved in dichloromethane and precipitated into diethyl ether. The product was isolated by filtration and dried in vacuo to give the block copolymer as an off-white powder. ¹H NMR (d₆-DMSO) δ 8.12-7.92, 4.58-4.40, 3.82-3.21, 1.83-1.14, 0.94-0.73 ppm

### Example 24

### Synthesis N₃-PEG12K-b-P(Asp(O^{t}Bu)₅)-b-P(D-Phe₇-co-Tyr(OBzl)₇)-Ac

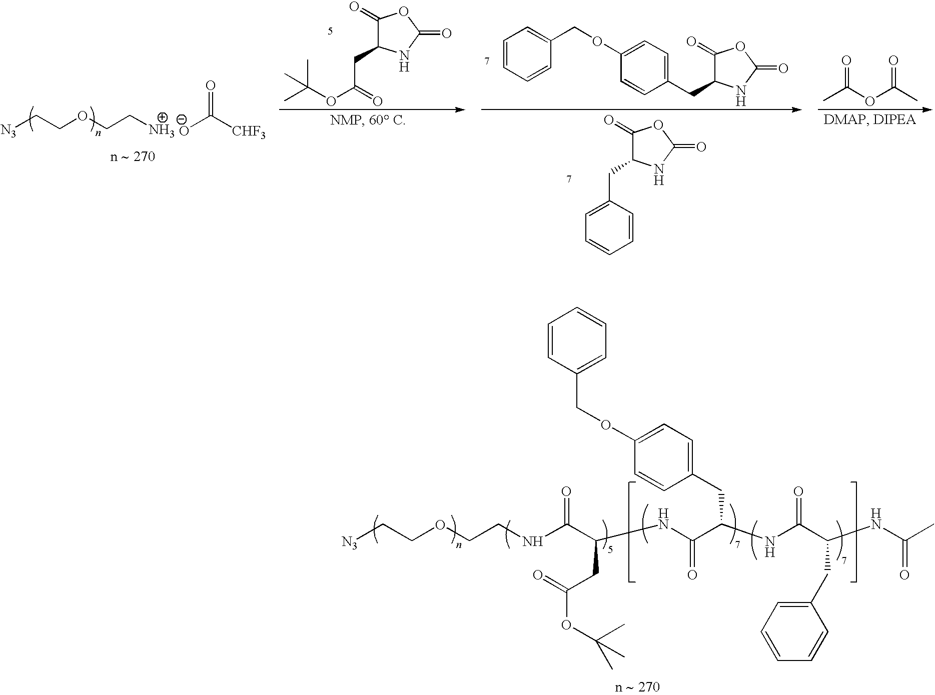

N₃-PEG12K-NH₂/DFA salt, (2.5 g, 0.21 mmol) was weighed into an oven-dried, round-bottom flask, dissolved in toluene, and dried by azeotropic distillation. Excess toluene was removed under vacuum. Asp(O^{t}Bu) NCA (0.22 g, 1.02 mmol) was added to the flask, the flask was evacuated under reduced pressure, and subsequently backfilled with nitrogen gas (repeated twice). Dry N-methylpyrrolidone (NMP) (13.6 mL) was introduced by syringe and the solution was heated to 60° C. The reaction mixture was allowed to stir for 15 hours at 60° C. under nitrogen gas. In an oven-dried round-bottom flask, D-Phe NCA (0.279 g, 1.46 mmol) and Tyr(OBzl) NCA (0.434 g, 25 mmol) were combined, 3 vacuum/N₂ cycles were applied and the white powder was dissolved in 3.6 ml of dry NMP under nitrogen gas. This solution was then transferred to the polymerization by syringe and allowed to stir for an additional 48 hours at 60° C. The solution was cooled to room temperature and DIPEA (1.0 mL), DMAP (100 mg), and acetic anhydride (1.0 mL) were added. Stirring was continued for 1 hour at room temperature. The polymer was precipitated into diethyl ether and isolated by filtration. The solid was redissolved in dichloromethane and precipitated into diethyl ether. The product was isolated by filtration and dried in vacuo to give 2.8 g (86% yield) of the block copolymer as an off-white powder. ¹H NMR (d₆-DMSO) δ 8.58-7.64, 7.42-6.58, 5.04-4.77, 4.72-4.23, 3.78-3.21, 3.04-2.75, 2.75-2.51, 2.51-2.34, 1.43-1.14 ppm

### Example 25

### Synthesis of N₃-PEG12K-b-P(Asp(O^{t}Bu)₅)-b-P(D-Phe₁₀-co-Tyr(OBzl)₁₀)-Ac

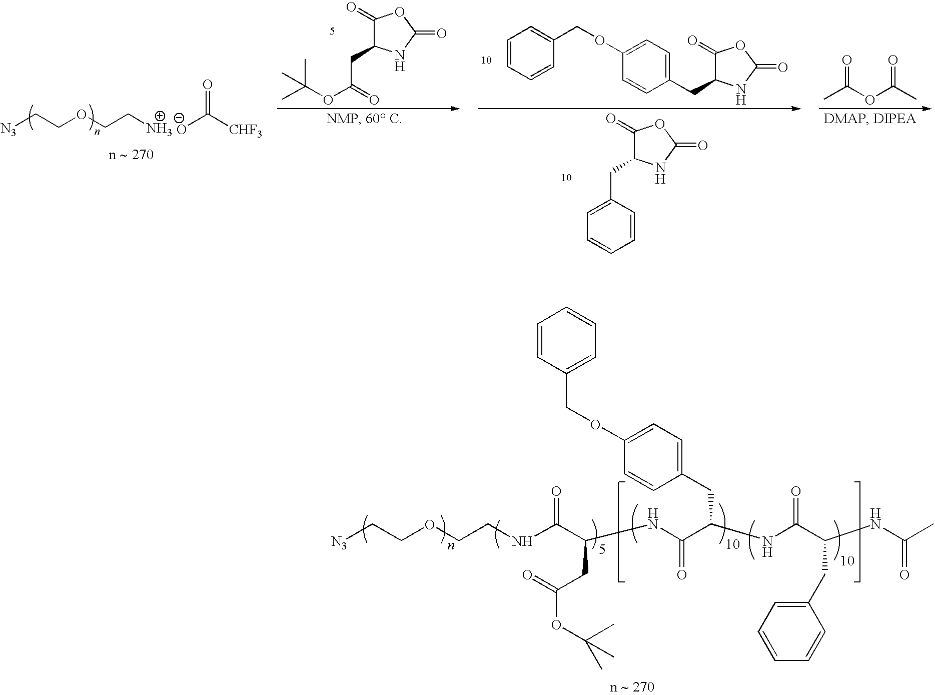

N₃-PEG12K-b-P(Asp(O^{t}Bu)₅)-b-P(D-Phe₁₀-co-Tyr(OBzl)₁₀)-Ac was synthesized as described in Example 24 from N₃-PEG-NH₂/DFA salt, 12 kDa (2.5 g, 0.21 mmol), Asp(O^{t}Bu) NCA (0.22 g, 1.02 mmol), D-Phe NCA (0.398 g, 2.1 mmol), Tyr(OBzl) NCA (0.691 g, 2.3 mmol) and 18.6 mL of NMP (13.6 mL for second block and 5 mL for third block). The block copolymer was isolated as an off-white powder (2.71 g, 77% yield). ¹H NMR (d₆-DMSO) δ 8.58-7.64, 7.42-6.58, 5.04-4.77, 4.72-4.23, 3.78-3.21, 3.04-2.75, 2.75-2.51, 2.51-2.34, 1.43-1.14 ppm

### Example 26

### Synthesis of N₃-PEG12K-b-P(Asp(O^{t}Bu)₅)-b-P(D-Phe₁₅-co-Tyr(OBzl)₁₅)-Ac

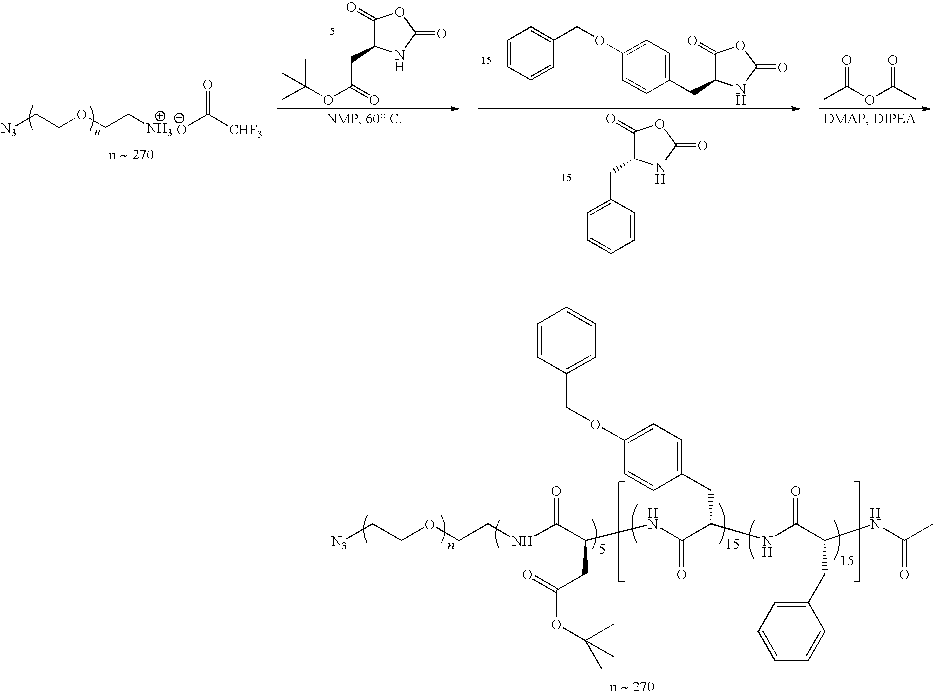

N₃-PEG 12K-b-P(Asp(O^{t}Bu)₅)-b-P(D-Phe₁₅-co-Tyr(OBzl)₁₅)-Ac was synthesized as described in Example 24 from N₃-PEG-NH₂/DFA salt, 12 kDa (2.5 g, 0.21 mmol), Asp(O^{t}Bu) NCA (0.22 g, 1.02 mmol), D-Phe NCA (0.597 g, 3.1 mmol), Tyr(OBzl) NCA (0.929 g, 3.1 mmol) and 21.2 mL of NMP (13.6 mL for second block and 7.6 mL for third block). The block copolymer was isolated as an off-white powder (3.49 g, 89% yield). ¹H NMR (d₆-DMSO) δ 8.58-7.64, 7.42-6.58, 5.04-4.77, 4.72-4.23, 3.78-3.21, 3.04-2.75, 2.75-2.51, 2.51-2.34, 1.43-1.14 ppm

### Example 27

### Synthesis of N₃-PEG12K-b-P(Asp(O^{t}Bu)₃)-b-P(D-Phe₇-co-Tyr(OBzl)₇)-Ac

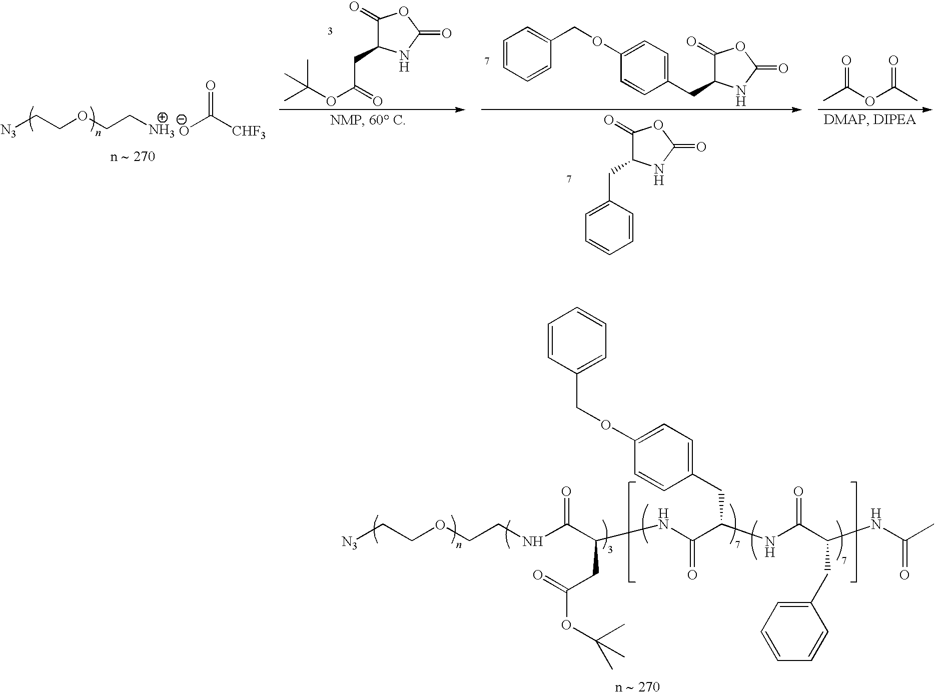

N₃-PEG12K-b-P(Asp(O^{t}Bu)₃)-b-P(D-Phe₇-co-Tyr(OBzl)₇)-Ac was synthesized as described in Example 24 from N₃-PEG-NH₂/DFA salt, 12 kDa (2.5 g, 0.21 mmol), Asp(O^{t}Bu) NCA (0.134 g, 0.62 mmol), D-Phe NCA (0.279 g, 1.46 mmol), Tyr(OBzl) NCA (0.434 g, 1.46 mmol) and 16.8 mL of NMP (13.2 mL for second block and 3.6 mL for third block). The block copolymer was isolated as an off-white powder (2.93 g, 92% yield). ¹H NMR (d₆-DMSO) δ 8.58-7.64, 7.42-6.58, 5.04-4.77, 4.72-4.23, 3.78-3.21, 3.04-2.75, 2.75-2.51, 2.51-2.34, 1.43-1.14 ppm

### Example 28

### Synthesis of N₃-PEG12K-b-P(Asp(O^{t}Bu)₇)-b-P(D-Phe₇-co-Tyr(OBzl)₇)-Ac

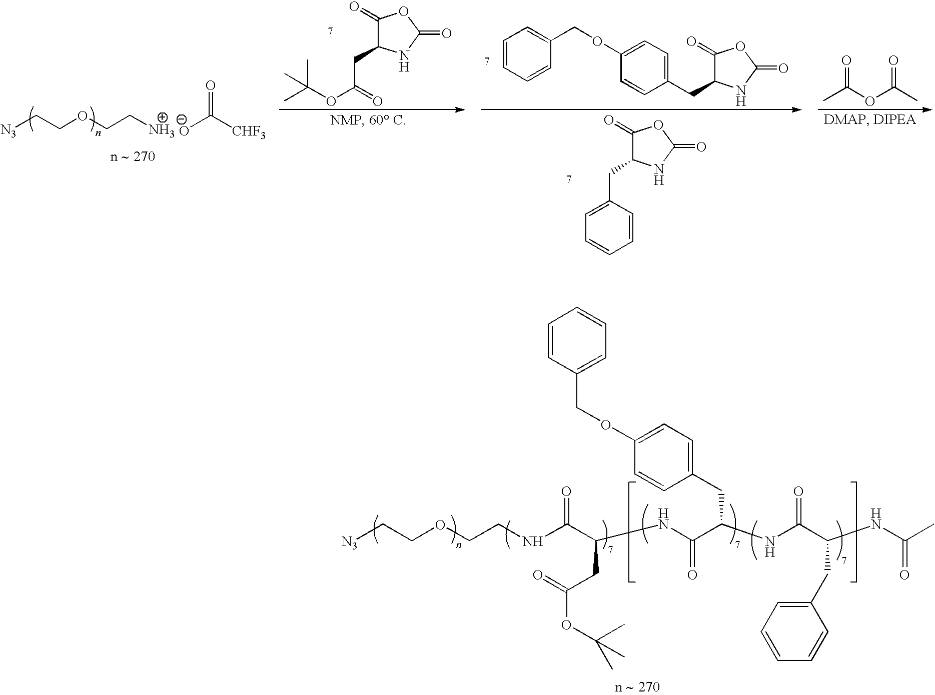

N₃-PEG12K-b-P(Asp(O^{t}Bu)₇)-b-P(D-Phe₇-co-Tyr(OBzl)₇)-Ac was synthesized as described in Example 24 from N₃-PEG-NH₂/DFA salt, 12 kDa (2.5 g, 0.21 mmol), Asp(O^{t}Bu) NCA (0.314 g, 1.46 mmol), D-Phe NCA (0.279 g, 1.46 mmol), Tyr(OBzl) NCA (0.434 g, 1.46 mmol) and 17.7 mL of NMP (14.1 mL for second block and 3.6 mL for third block). The block copolymer was isolated as an off-white powder (2.80 g, 84% yield). ¹H NMR (d₆-DMSO) δ 8.58-7.64, 7.42-6.58, 5.04-4.77, 4.72-4.23, 3.78-3.21, 3.04-2.75, 2.75-2.51, 2.51-2.34, 1.43-1.14 ppm

### Example 29

### Synthesis of N₃-PEG12K-b-P(Asp(O^{t}Bu)₁₀)-b-P(D-Phe₇-co-Tyr(OBzl)₇)-Ac

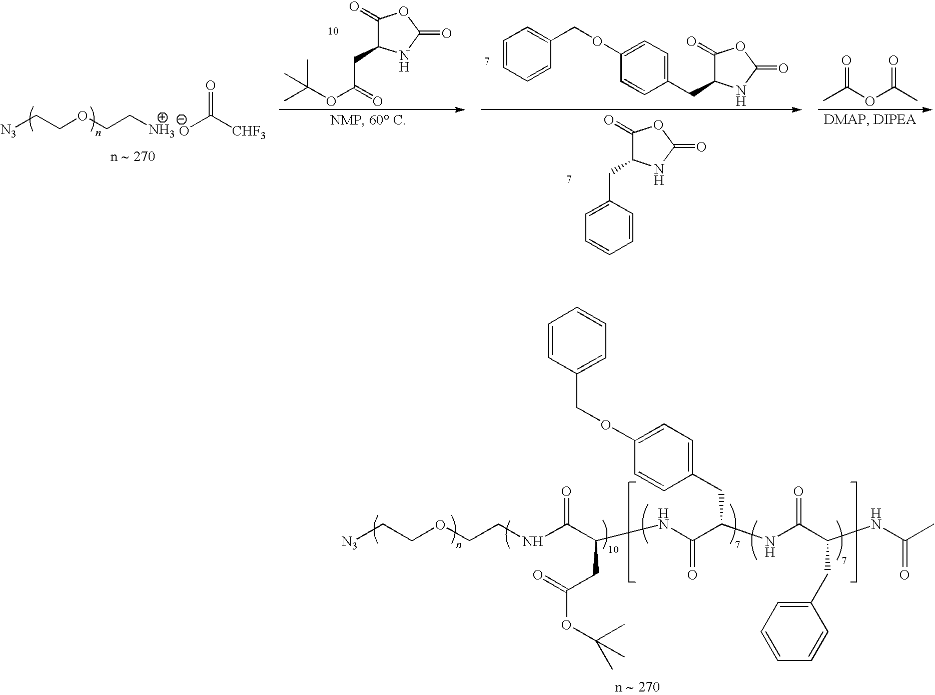

N₃-PEG12K-b-P(Asp(O^{t}Bu)₁₀)-b-P(D-Phe₇-co-Tyr(OBzl)₇)-Ac was synthesized as described in Example 24 from N₃-PEG-NH₂/DFA salt, 12 kDa (2.5 g, 0.21 mmol), Asp(O^{t}Bu) NCA (0.448 g, 2.1 mmol), D-Phe NCA (0.279 g, 1.46 mmol), Tyr(OBzl) NCA (0.434 g, 1.46 mmol) and 18.3 mL of NMP (14.7 mL for second block and 3.6 mL for third block). The block copolymer was isolated as an off-white powder (2.45 g, 71% yield). ¹H NMR (d₆-DMSO) δ 8.58-7.64, 7.42-6.58, 5.04-4.77, 4.72-4.23, 3.78-3.21, 3.04-2.75, 2.75-2.51, 2.51-2.34, 1.43-1.14 ppm

### Example 30

### Synthesis of N₃-PEG12K-b-P(Asp(O^{t}Bu)₁₀)-b-P(D-Phe₁₀-co-Tyr(OBzl)₁₀)-Ac

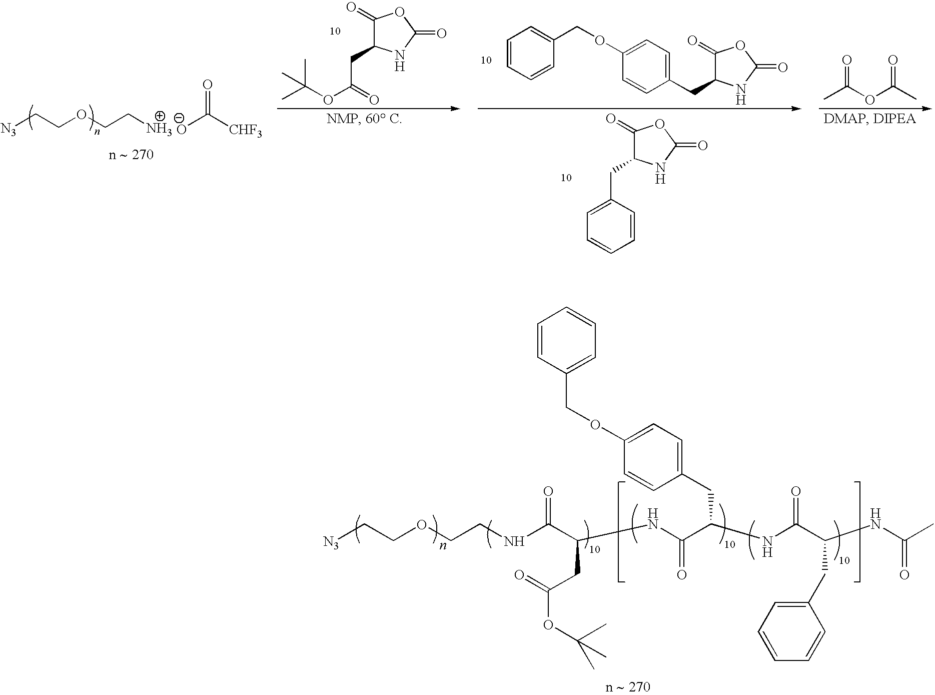

N₃-PEG12K-b-P(Asp(O^{t}Bu)₁₀)-b-P(D-Phe₁₀-co-Tyr(OBzl)₁₀)-Ac was synthesized as described in Example 24 from N₃-PEG-NH₂/DFA salt, 12 kDa (20 g, 1.67 mmol), Asp(O^{t}Bu) NCA (3.59 g, 16.7 mmol), D-Phe NCA (3.19 g, 16.7 mmol), Tyr(OBzl) NCA (4.96 g, 16.7 mmol) and 165 mL of NMP (125 mL for second block and 40 mL for third block). The block copolymer was isolated as an off-white powder (22.5 g, 76% yield). ¹H NMR (d₆-DMSO) δ 8.58-7.64, 7.42-6.58, 5.04-4.77, 4.72-4.23, 3.78-3.21, 3.04-2.75, 2.75-2.51, 2.51-2.34, 1.43-1.14 ppm

### Example 31

### Synthesis of N₃-PEG12K-b-P(Asp(O^{t}Bu)₂₅-co-D-Leu₅₀-co-Orn(Z)₂₅)-Ac

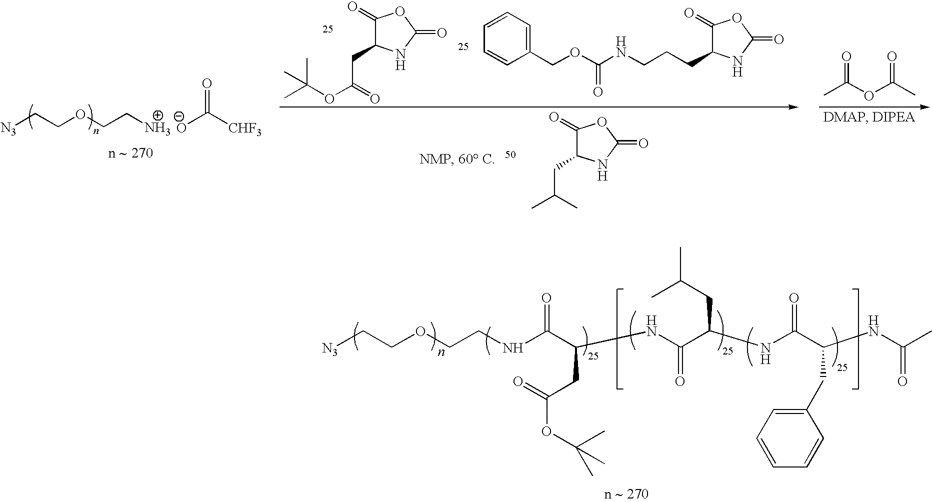

N₃-PEG12K-NH₂/DFA salt, (10.0 g, 0.83 mmol) was weighed into an oven-dried, round-bottom flask, dissolved in toluene, and dried by azeotropic distillation. Excess toluene was removed under vacuum. Asp(O^{t}Bu) NCA (2.15 g, 10 mmol), D-Leu NCA (6.55 g, 41.7 mmol) and Orn(Z) NCA (5.02 g, 17.2 mmol) was added to the flask, the flask was evacuated under reduced pressure, and subsequently backfilled with nitrogen gas. Dry N-methylpyrrolidone (NMP) (130 mL) was introduced by syringe and the solution was heated to 60° C. The reaction mixture was allowed to stir for 5 days at 60° C. under nitrogen gas. The solution was cooled to room temperature and DIPEA (2.0 mL), DMAP (100 mg), and acetic anhydride (2.0 mL) were added. Stirring was continued for 1 hour at room temperature. The polymer was precipitated into diethyl ether (cooled down to −20° C.) and isolated by filtration. The solid was re-dissolved in dichloromethane and precipitated into diethyl ether (cooled down to −20° C.). The product was isolated by filtration and dried in vacuo to give the block copolymer as an off-white powder. ¹H NMR (d₆-DMSO) δ 8.44-7.58, 7.38-7.08, 5.04-4.89, 4.63-4.38, 4.35-4.14, 3.50, 3.05-2.88, 2.75-2.61, 2.48, 1.5-1.15, 0.95-0.71 ppm

### Example 32

### Synthesis of N₃-PEG12K-b-P(Asp(O^{t}Bu)₅₀)-Ac

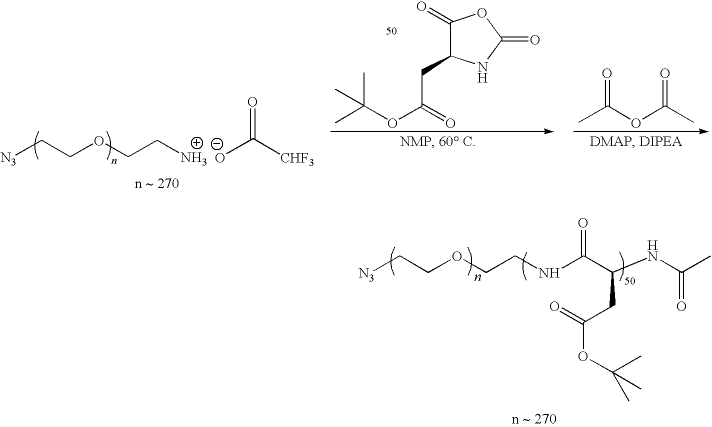

N₃-PEG12K-b-P(Asp(O^{t}Bu)₅₀)-Ac was synthesized as described in Example 31 from N₃-PEG-NH₂/DFA salt, 12 kDa (5 g, 0.42 mmol), Asp(O^{t}Bu) NCA (4.48 g, 20.8 mmol) and 47 mL of NMP. The block copolymer was isolated as an off-white powder. ¹H NMR (d₆-DMSO) δ 8.12-7.90, 4.63-4.43, 3.90-3.04, 2.64-2.37, 1.37 ppm

### Example 33

### Synthesis of N₃-PEG12K-b-P(Asp(O^{t}Bu)₅₀-co-D-Leu₅₀)-Ac

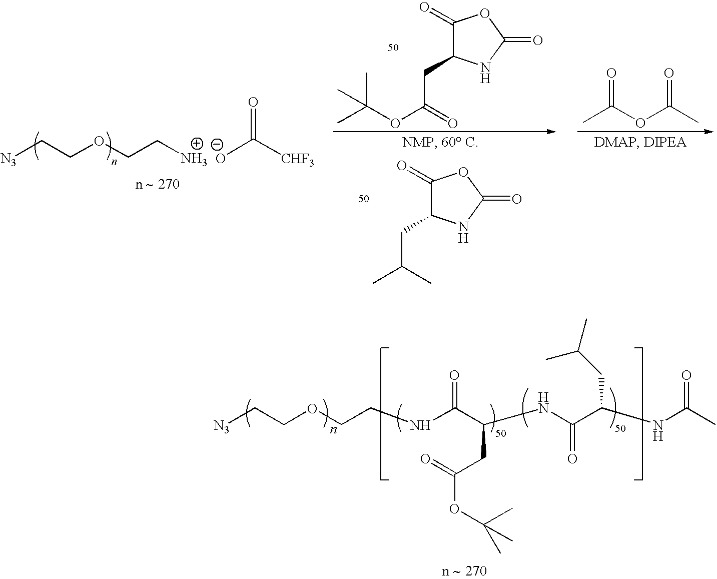

N₃-PEG12K-b-P(Asp(O^{t}Bu)₅₀-co-D-Leu₅₀)-Ac was synthesized as described in Example 31 from N₃-PEG-NH₂/DFA salt, 12 kDa (5 g, 0.42 mmol), Asp(O^{t}Bu) NCA (4.48 g, 20.8 mmol), D-Leu NCA (3.27 g, 20.8 mmol) and 64 mL of NMP. The block copolymer was isolated as an off-white powder. ¹H NMR (d₆-DMSO) δ 8.12-7.92, 4.58-4.40, 3.82-3.21, 1.83-1.14, 0.94-0.73 ppm

### Example 34

### Synthesis of N₃-PEG12K-b-P(Asp(O^{t}Bu)₁₀₀-co-D-Leu₁₀₀)-Ac

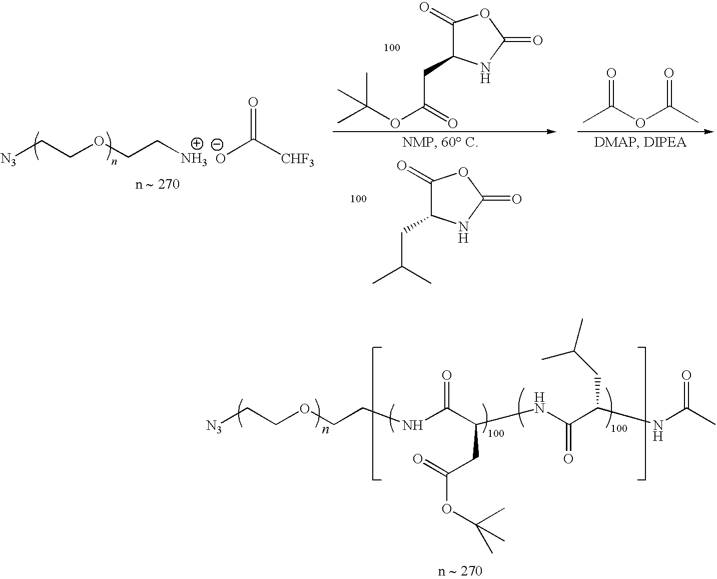

N₃-PEG12K-b-P(Asp(O^{t}Bu)₁₀₀-co-D-Leu₁₀₀)-Ac was synthesized as described in Example 31 from N₃-PEG-NH₂/DFA salt, 12 kDa (5 g, 0.42 mmol), Asp(O^{t}Bu) NCA (8.97 g, 41.6 mmol), D-Leu NCA (6.55 g, 41.6 mmol) and 103 mL of NMP. The block copolymer was isolated as an off-white powder. ¹H NMR (d₆-DMSO) δ 8.12-7.92, 4.58-4.40, 3.82-3.21, 1.83-1.14, 0.94-0.73 ppm

### Example 35

### Synthesis of N₃-PEG5K-b-P(Asp(O^{t}Bu)₅₀-co-D-Leu₂₅-co-Orn(Z)₅₀)-Ac

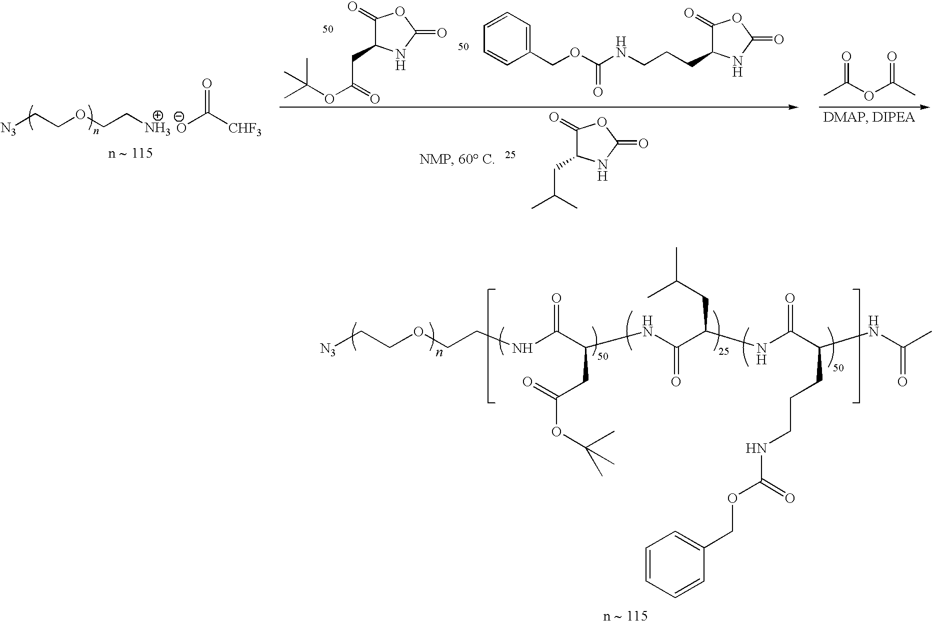

N₃-PEG5K-b-P(Asp(O^{t}Bu)₅₀-co-D-Leu₂₅-co-Orn(Z)₅₀)-Ac was synthesized as described in Example 31 from N₃-PEG-NH₂/DFA salt, 5 kDa (0.5 g, 0.1 mmol), Asp(O^{t}Bu) NCA (1.08 g, 5 mmol), D-Leu NCA (0.39 g, 2.5 mmol), Orn(Z) NCA (1.46 g, 5 mmol) and 23 mL of NMP. The block copolymer was isolated as an off-white powder (1.6 g, 56% yield). ¹H NMR (d₆-DMSO) δ 8.44-7.58, 7.38-7.08, 5.04-4.89, 4.63-4.38, 4.35-4.14, 3.50, 3.05-2.88, 2.75-2.61, 2.48, 1.75-1.15, 0.95-0.71 ppm

### Example 36

### Synthesis of N₃-PEG5K-b-P(Asp(O^{t}Bu)₇₅-co-D-Leu₂₅-co-Orn(Z)₅₀)-Ac

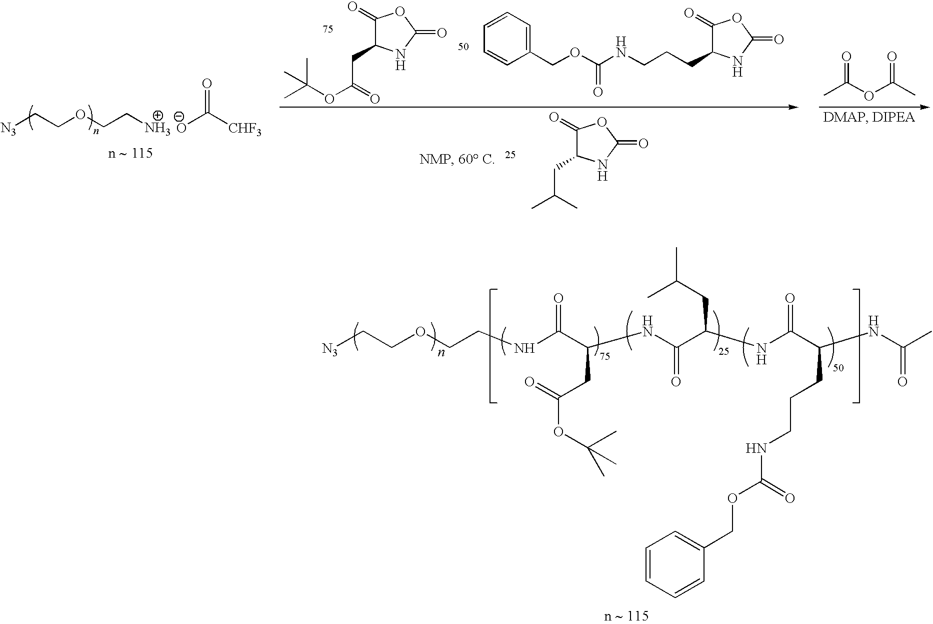

N₃-PEG5K-b-P(Asp(O^{t}Bu)₇₅-co-D-Leu₂₅-co-Orn(Z)₅₀)-Ac was synthesized as described in Example 31 from N₃-PEG-NH₂/DFA salt, 5 kDa (0.5 g, 0.1 mmol), Asp(O^{t}Bu) NCA (1.61 g, 7.5 mmol), D-Leu NCA (0.39 g, 2.5 mmol), Orn(Z) NCA (1.46 g, 5 mmol) and 26 mL of NMP. The block copolymer was isolated as an off-white powder (1.3 g, 39% yield). ¹H NMR (d₆-DMSO) δ 8.44-7.58, 7.38-7.08, 5.04-4.89, 4.63-4.38, 4.35-4.14, 3.50, 3.05-2.88, 2.75-2.61, 2.48, 1.75-1.15, 0.95-0.71 ppm

### Example 37

### Synthesis of N₃-PEG5K-b-P(Asp(O^{t}Bu)₁₀₀-co-D-Leu₂₅-co-Orn(Z)₅₀)-Ac

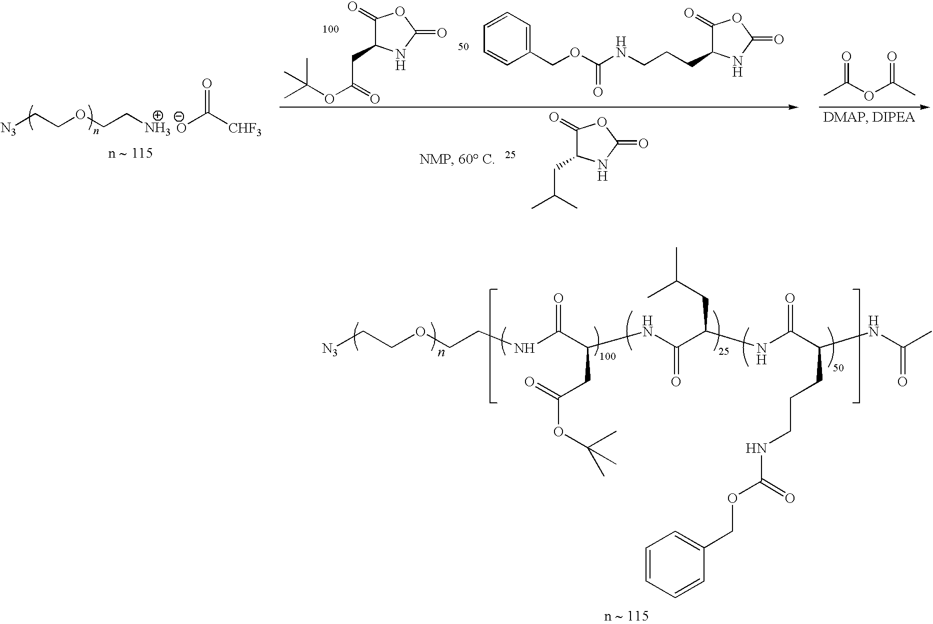

N₃-PEG5K-b-P(Asp(O^{t}Bu)100-co-D-Leu₂₅-co-Orn(Z)₅₀)-Ac was synthesized as described in Example 31 from N₃-PEG-NH₂/DFA salt, 5 kDa (0.5 g, 0.1 mmol), Asp(O^{t}Bu) NCA (2.15 g, 10 mmol), D-Leu NCA (0.39 g, 2.5 mmol), Orn(Z) NCA (1.46 g, 5 mmol) and 30 mL of NMP. The block copolymer was isolated as an off-white powder (1.9 g, 51% yield). ¹H NMR (d₆-DMSO) δ 8.44-7.58, 7.38-7.08, 5.04-4.89, 4.63-4.38, 4.35-4.14, 3.50, 3.05-2.88, 2.75-2.61, 2.48, 1.75-1.15, 0.95-0.71 ppm

### Example 38

### Synthesis of N₃-PEG5K-b-P(Asp(O^{t}Bu)₁₀₀-co-D-Leu₂₅-co-Orn(Z)₁₀₀)-Ac

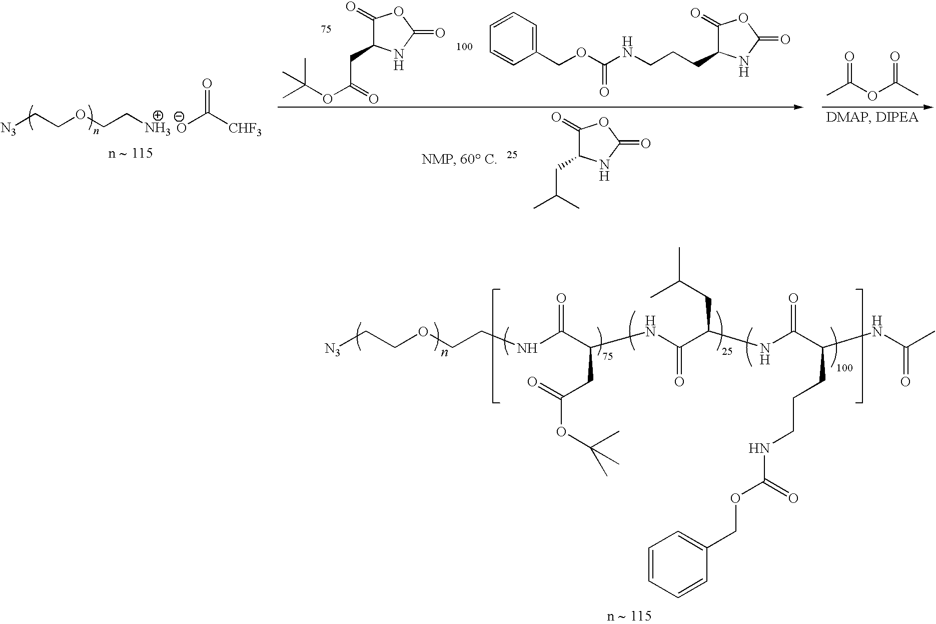

N₃-PEG5K-b-P(Asp(O^{t}Bu)100-co-D-Leu₂₅-co-Orn(Z)₁₀₀)-Ac was synthesized as described in Example 31 from N₃-PEG-NH₂/DFA salt, 5 kDa (0.5 g, 0.1 mmol), Asp(O^{t}Bu) NCA (2.15 g, 10 mmol), D-Leu NCA (0.39 g, 2.5 mmol), Orn(Z) NCA (2.92 g, 10 mmol) and 40 mL of NMP. The block copolymer was isolated as an off-white powder. ¹H NMR (d₆-DMSO) δ 8.44-7.58, 7.38-7.08, 5.04-4.89, 4.63-4.38, 4.35-4.14, 3.50, 3.05-2.88, 2.75-2.61, 2.48, 1.75-1.15, 0.95-0.71 ppm

### Example 39

### Synthesis of N₃-PEG5K-b-P(Asp(O^{t}Bu)₇₅-co-D-Leu₂₅-co-Orn(Z)₁₀₀)-Ac

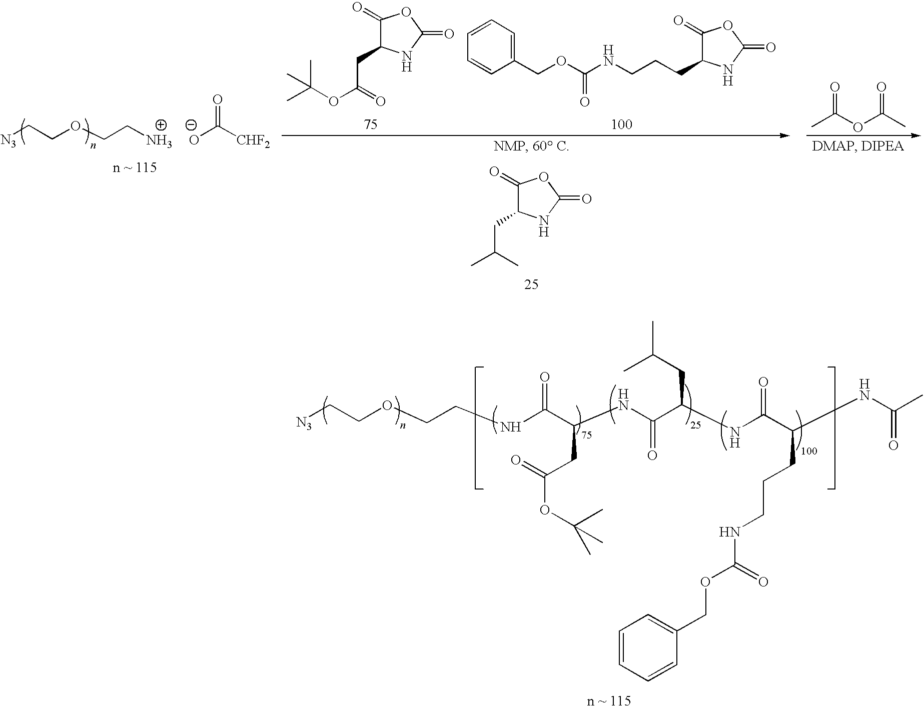

N₃-PEG5K-b-P(Asp(O^{t}Bu)₇₅-co-D-Leu₂₅-co-Orn(Z)₁₀₀)-Ac was synthesized as described in Example 31 from N₃-PEG-NH₂/DFA salt, 5 kDa (0.5 g, 0.1 mmol), Asp(O^{t}Bu) NCA (1.61 g, 7.5 mmol), D-Leu NCA (0.39 g, 2.5 mmol), Orn(Z) NCA (2.92 g, 10 mmol) and 36 mL of NMP. The block copolymer was isolated as an off-white powder. ¹H NMR (d₆-DMSO) δ 8.44-7.58, 7.38-7.08, 5.04-4.89, 4.63-4.38, 4.35-4.14, 3.50, 3.05-2.88, 2.75-2.61, 2.48, 1.75-1.15, 0.95-0.71 ppm

### Example 40

### Synthesis of N₃-PEG5K-b-P(Asp(O^{t}Bu)₁₀₀-co-D-Leu₅₀-co-Orn (Z)₅₀)-Ac

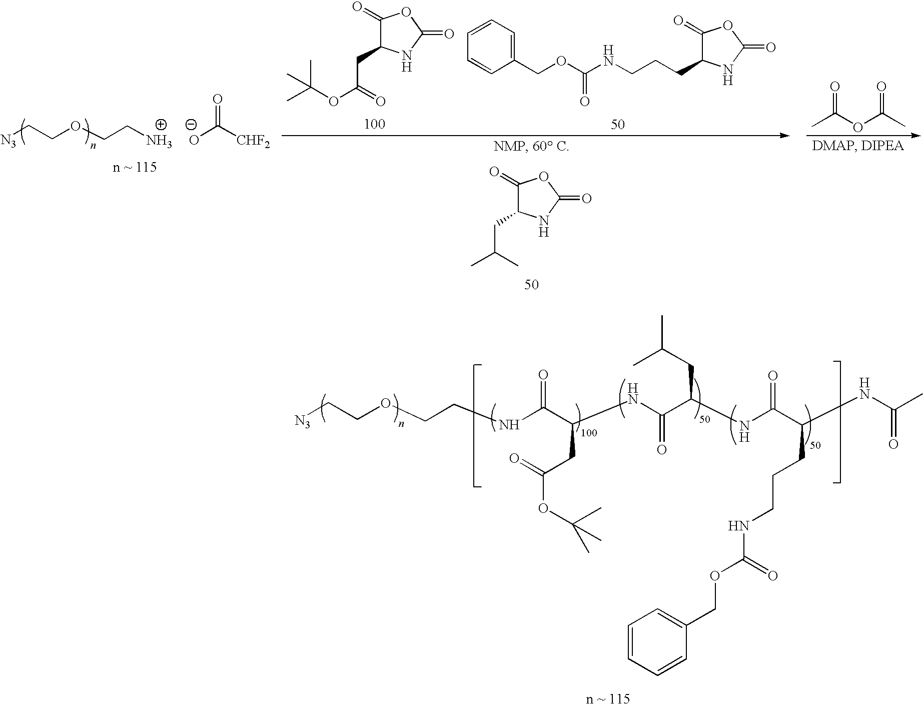

N₃-PEG5K-b-P(Asp(O^{t}Bu)₁₀₀-co-D-Leu₅₀-co-Orn(Z)₅₀)-Ac was synthesized as described in Example 31 from N₃-PEG-NH₂/DFA salt, 5 kDa (0.5 g, 0.1 mmol), Asp(O^{t}Bu) NCA (2.15 g, 10 mmol), D-Leu NCA (0.79 g, 5 mmol), Orn(Z) NCA (1.46 g, 5 mmol) and 33 mL of NMP. The block copolymer was isolated as an off-white powder (2.52 g, 63% yield). ¹H NMR (d₆-DMSO) δ 8.44-7.58, 7.38-7.08, 5.04-4.89, 4.63-4.38, 4.35-4.14, 3.50, 3.05-2.88, 2.75-2.61, 2.48, 1.75-1.15, 0.95-0.71 ppm

### Example 41

### Synthesis of N₃-PEG5K-b-P(Asp(O^{t}Bu)₅₀-b-P(D-Leu₅₀-co-Orn (Z)₅₀)-Ac

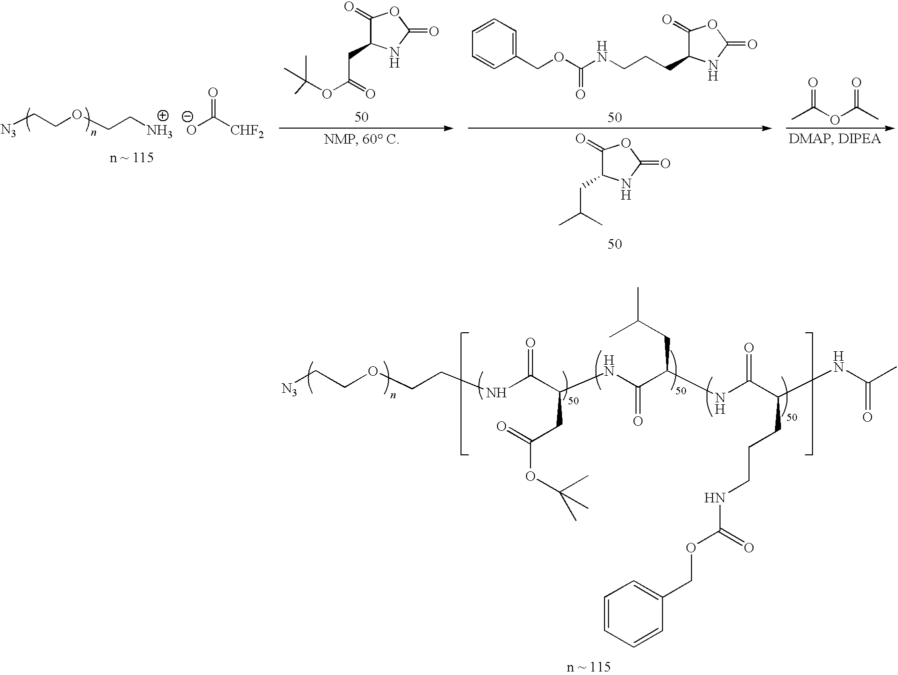

N₃-PEG5k-NH₂/DFA salt, (0.5 g, 0.1 mmol) was weighed into an oven-dried, round-bottom flask, dissolved in toluene, and dried by azeotropic distillation. Excess toluene was removed under vacuum. Asp(O^{t}Bu) NCA (1.08 g, 5 mmol) was added to the flask, the flask was evacuated under reduced pressure, and subsequently backfilled with nitrogen gas (repeated twice). Dry N-methylpyrrolidone (NMP) (10.5 mL) was introduced by syringe and the solution was heated to 60° C. The reaction mixture was allowed to stir for 2 days at 60° C. under nitrogen gas. In an oven-dried 2-neck round-bottom flask, D-Leu NCA (0.79 g, 5 mmol) and Orn(Z) NCA (1.46 g, 5 mmol) were combined, 3 vacuum/N₂ cycles were applied and the white powder was dissolved in 15 ml of dry NMP under nitrogen gas. This solution was then transferred to the polymerization by syringe and allowed to stir for an additional 4 days 15 h at 60° C. The solution was cooled to room temperature and DIPEA (1.0 mL), DMAP (100 mg), and acetic anhydride (1.0 mL) were added. Stirring was continued for 1 hour at room temperature. The polymer was precipitated into diethyl ether and isolated by filtration. The solid was re-dissolved in dichloromethane and precipitated into diethyl ether. The product was isolated by filtration and dried in vacuo to give 2.39 g (75% yield) of the block copolymer as an off-white powder. ¹H NMR (d₆-DMSO) δ 8.44-7.58, 7.38-7.08, 5.04-4.89, 4.63-4.38, 4.35-4.14, 3.50, 3.05-2.88, 2.75-2.61, 2.48, 1.75-1.15, 0.95-0.71 ppm

### Example 42

### Synthesis N₃-PEG5K-b-P(Asp(O^{t}Bu)₇₅-b-P(D-Leu₅₀-co-Orn (Z)₅₀)-Ac

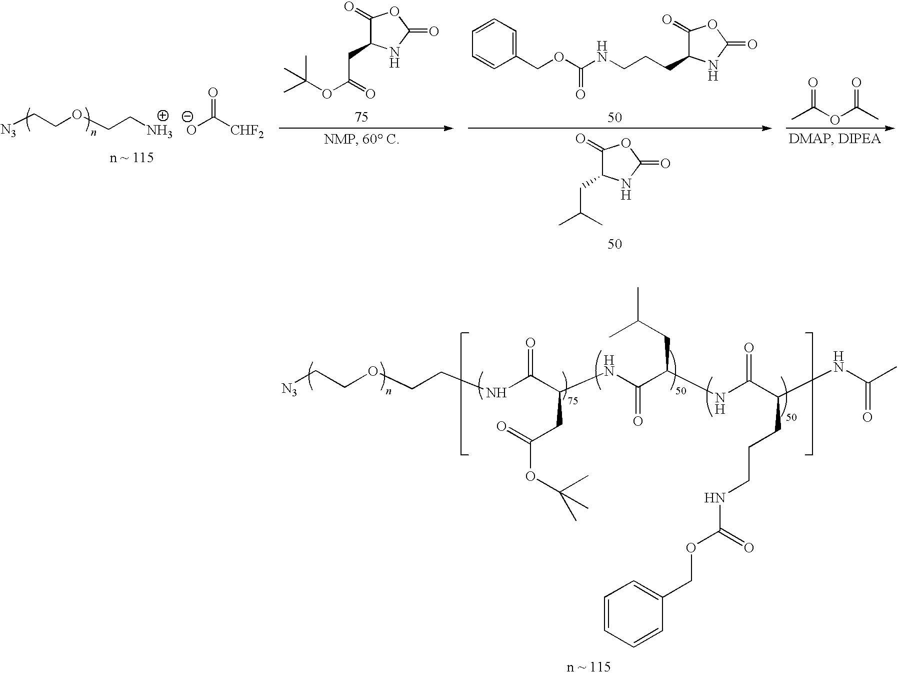

N₃-PEG5K-b-P(Asp(O^{t}Bu)₇₅-b-P(D-Leu₅₀-co-Orn(Z)₅₀)-Ac was synthesized as described in Example 41 from N₃-PEG-NH₂/DFA salt, 5 kDa (0.5 g, 0.1 mmol), Asp(O^{t}Bu) NCA (1.61 g, 7.5 mmol), D-Leu NCA (0.79 g, 5 mmol), Orn(Z) NCA (1.46 g, 5 mmol) and 36 mL of NMP (21 mL of NMP for the second block and 15 mL for the third block). The block copolymer was isolated as an off-white powder (2.7 g, 75% yield). ¹H NMR (d₆-DMSO) δ 8.44-7.58, 7.38-7.08, 5.04-4.89, 4.63-4.38, 4.35-4.14, 3.50, 3.05-2.88, 2.75-2.61, 2.48, 1.75-1.15, 0.95-0.71 ppm

### Example 43

### Synthesis of N₃-PEG5K-b-P(Asp(O^{t}Bu)₁₀₀-b-P(D-Leu₅₀-co-Orn(Z)₅₀)-Ac

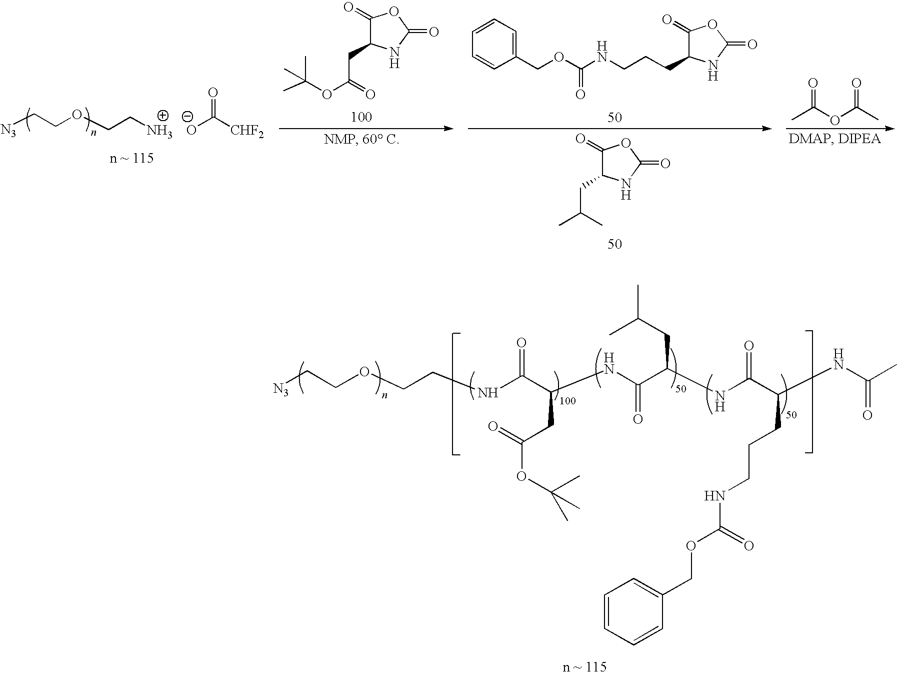

N₃-PEG5K-b-P(Asp(O^{t}Bu)₁₀₀-b-P(D-Leu₅₀-co-Orn(Z)₅₀)-Ac was synthesized as described in Example 41 from N₃-PEG-NH₂/DFA salt, 5 kDa (0.5 g, 0.1 mmol), Asp(O^{t}Bu) NCA (2.15 g, 10 mmol), D-Leu NCA (0.79 g, 5 mmol), Orn(Z) NCA (1.46 g, 5 mmol) and 41 mL of NMP (26 mL NMP for the second block and 15 mL for the third block). The block copolymer was isolated as an off-white powder (1.86 g, 46% yield). ¹H NMR (d₆-DMSO) δ 8.44-7.58, 7.38-7.08, 5.04-4.89, 4.63-4.38, 4.35-4.14, 3.50, 3.05-2.88, 2.75-2.61, 2.48, 1.75-1.15, 0.95-0.71 ppm

### Example 44

### Synthesis of N₃-PEG5K-b-P(Asp(OBzl)₅₀)-Ac

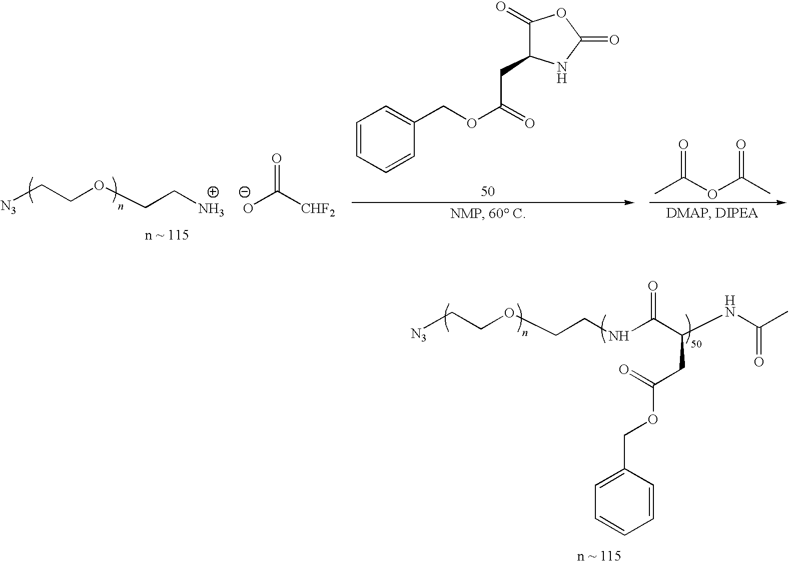

N₃-PEG5K-NH₂/DFA salt, (1 g, 0.2 mmol) was weighed into an oven-dried, round-bottom flask, dissolved in toluene, and dried by azeotropic distillation. Excess toluene was removed under vacuum. Asp(O^{t}Bu) NCA (2.49 g, 10 mmol) was added to the flask, the flask was evacuated under reduced pressure, and subsequently backfilled with nitrogen gas (repeated twice). Dry N-methylpyrrolidone (NMP) (17.5 mL) was introduced by syringe and the solution was heated to 60° C. The reaction mixture was allowed to stir for 2 days at 60° C. under nitrogen gas. The solution was cooled to room temperature and DIPEA (1.0 mL), DMAP (100 mg), and acetic anhydride (1.0 mL) were added. Stirring was continued for 1 hour at room temperature. The polymer was then placed in a 3500 g/mol molecular weight cut-off dialysis bag, dialyzed three times against 0.1N methanol, three times against deionized water and freeze-dried. A white solid was obtained (2.03 g, 66% yield). ¹H NMR (d₆-DMSO) δ 8.54-8.09, 7.44-7.17, 5.23-4.88, 4.63-4.43, 3.63, 3.25, 2.89-2.69, 2.67-2.54 ppm.

### Example 45

### Synthesis of N₃-PEG5K-b-P(Asp(OBzl)₇₅)-Ac

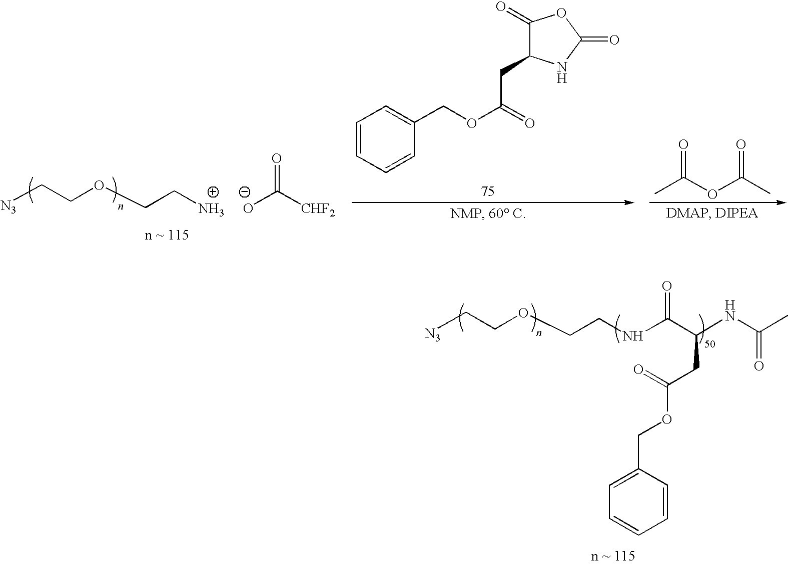

N₃-PEG5K-b-P(Asp(OBzl)₇₅)-Ac was synthesized as described in Example 44 from N₃-PEG-NH₂/DFA salt, 5 kDa (1 g, 0.2 mmol), Asp(O^{t}Bu) NCA (3.74 g, 15 mmol) and 48 mL of NMP. The block copolymer was isolated as an off-white powder. ¹H NMR (d₆-DMSO) δ 8.54-8.09, 7.44-7.17, 5.23-4.88, 4.63-4.43, 3.63, 3.25, 2.89-2.69, 2.67-2.54 ppm.

### Example 46

### Synthesis of N₃-PEG5K-b-P(Asp(OBzl)₁₀₀)-Ac

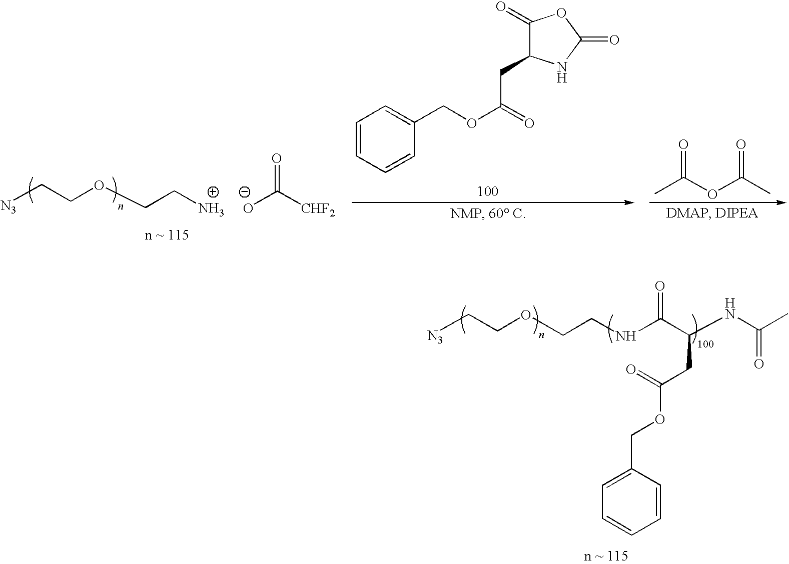

N₃-PEG5K-b-P(Asp(OBzl)₁₀₀)-Ac was synthesized as described in Example 44 from N₃-PEG-NH₂/DFA salt, 5 kDa (1 g, 0.2 mmol), Asp(O^{t}Bu) NCA (4.98 g, 20 mmol) and 60 mL of NMP. The block copolymer was isolated as an off-white powder. ¹H NMR (d₆-DMSO) δ 8.54-8.09, 7.44-7.17, 5.23-4.88, 4.63-4.43, 3.63, 3.25, 2.89-2.69, 2.67-2.54 ppm.

### Example 47

### Synthesis of N₃-PEG5K-b-P(Asp(OBzl)₂₅-co-D-Asp(OBzl)₂₅)-Ac

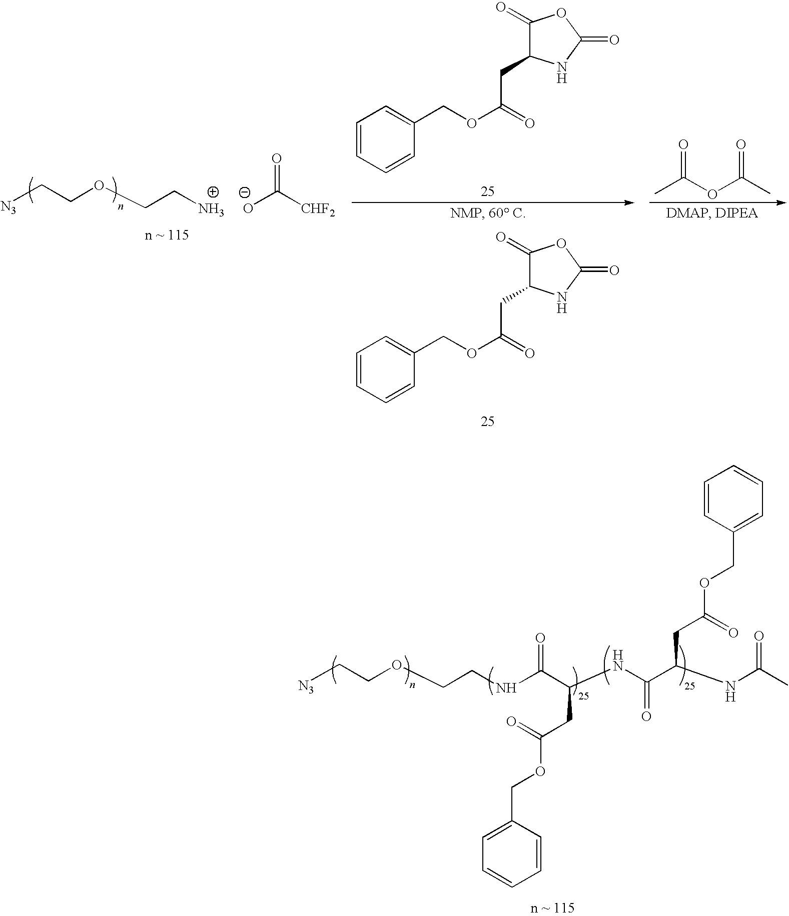

N₃-PEG5K-b-P(Asp(OBzl)₂₅-co-D-Asp(OBzl)₂₅)-Ac was synthesized as described in Example 44 from N₃-PEG-NH₂/DFA salt, 5 kDa (1 g, 0.2 mmol), Asp(O^{t}Bu) NCA (1.25 g, 5 mmol), D-Asp(O^{t}Bu) NCA (1.25 g, 5 mmol) and 18 mL of NMP. The block copolymer was isolated as an off-white powder. ¹H NMR (d₆-DMSO) δ 8.54-8.09, 7.44-7.17, 5.23-4.88, 4.63-4.43, 3.63, 3.25, 2.89-2.69, 2.67-2.54 ppm.

### Example 48

### Synthesis of N₃-PEG5K-b-P(Asp(OBzl)₃₇-co-D-Asp(OBzl)₃₇)-Ac

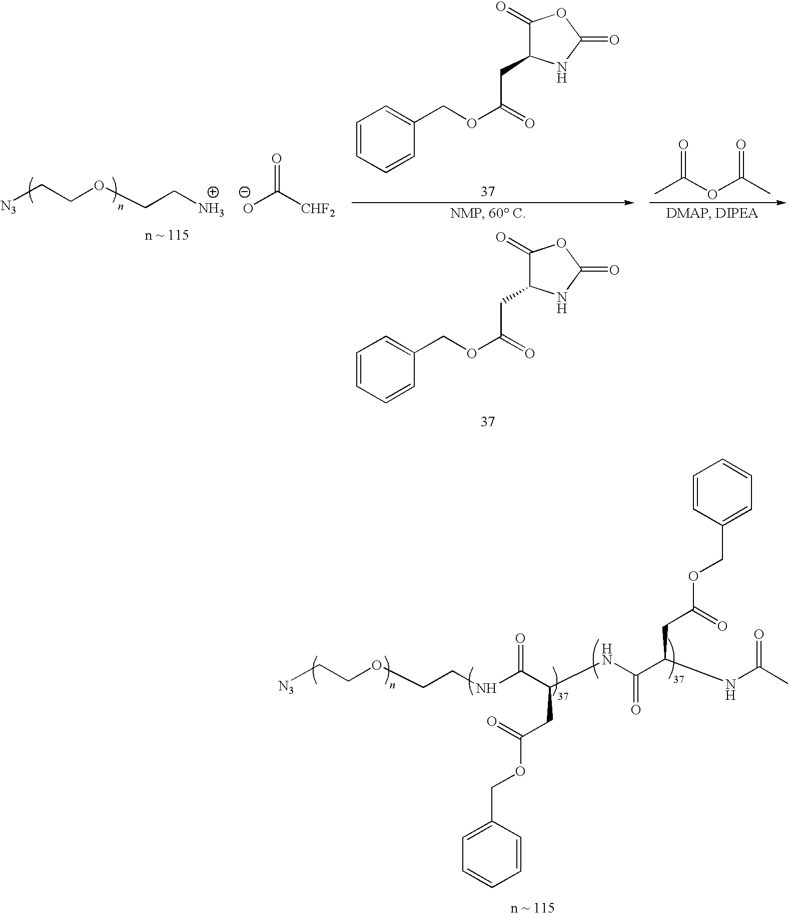

N₃-PEG5K-b-P(Asp(OBzl)₃₇-co-D-Asp(OBzl)₃₇)-Ac was synthesized as described in Example 44 from N₃-PEG-NH₂/DFA salt, 5 kDa (1 g, 0.2 mmol), Asp(O^{t}Bu) NCA (1.84 g, 7.4 mmol), D-Asp(O^{t}Bu) NCA (1.84 g, 7.4 mmol) and 47 mL of NMP. The block copolymer was isolated as an off-white powder. ¹H NMR (d₆-DMSO) δ 8.54-8.09, 7.44-7.17, 5.23-4.88, 4.63-4.43, 3.63, 3.25, 2.89-2.69, 2.67-2.54 ppm.

### Example 49

### Synthesis of N₃-PEG5K-b-P(Asp(OBzl)₅₀-co-D-Asp(OBzl)₅₀)-Ac

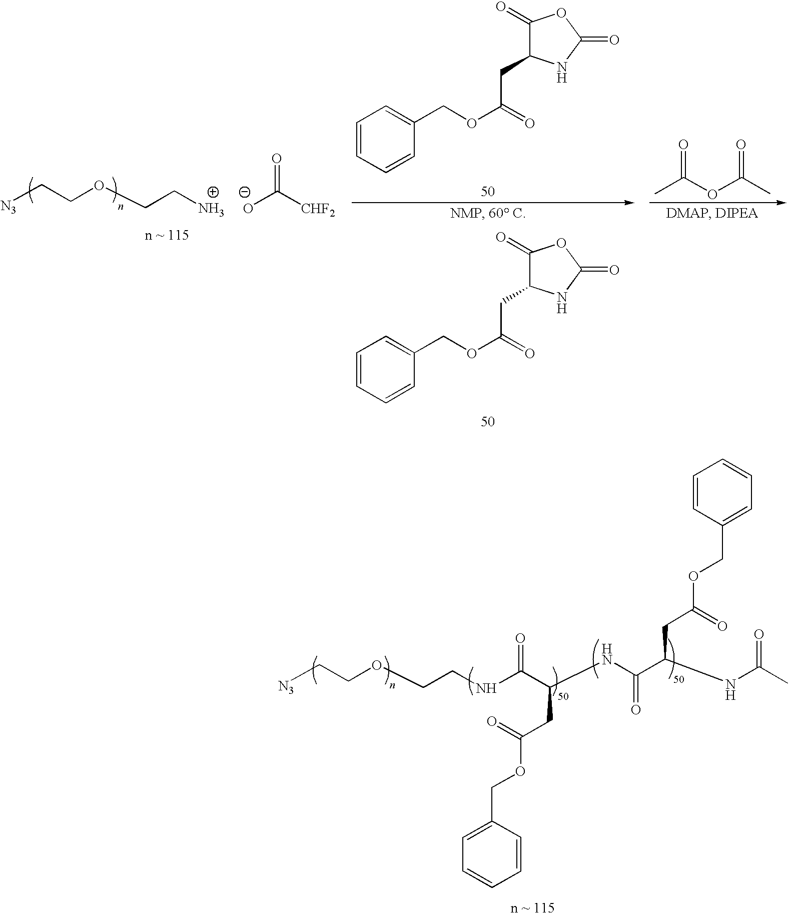

N₃-PEG5K-b-P(Asp(OBzl)₅₀-co-D-Asp(OBzl)₅₀)-Ac was synthesized as described in Example 44 from N₃-PEG-NH₂/DFA salt, 5 kDa (1 g, 0.2 mmol), Asp(O^{t}Bu) NCA (2.49 g, 10 mmol), D-Asp(O^{t}Bu) NCA (2.49 g, 10 mmol) and 60 mL of NMP. The block copolymer was isolated as an off-white powder. ¹H NMR (d₆-DMSO) δ 8.54-8.09, 7.44-7.17, 5.23-4.88, 4.63-4.43, 3.63, 3.25, 2.89-2.69, 2.67-2.54 ppm.

### Example 50

### Synthesis of N₃-PEG5K-b-P(Orn(Z)₅₀)-Ac

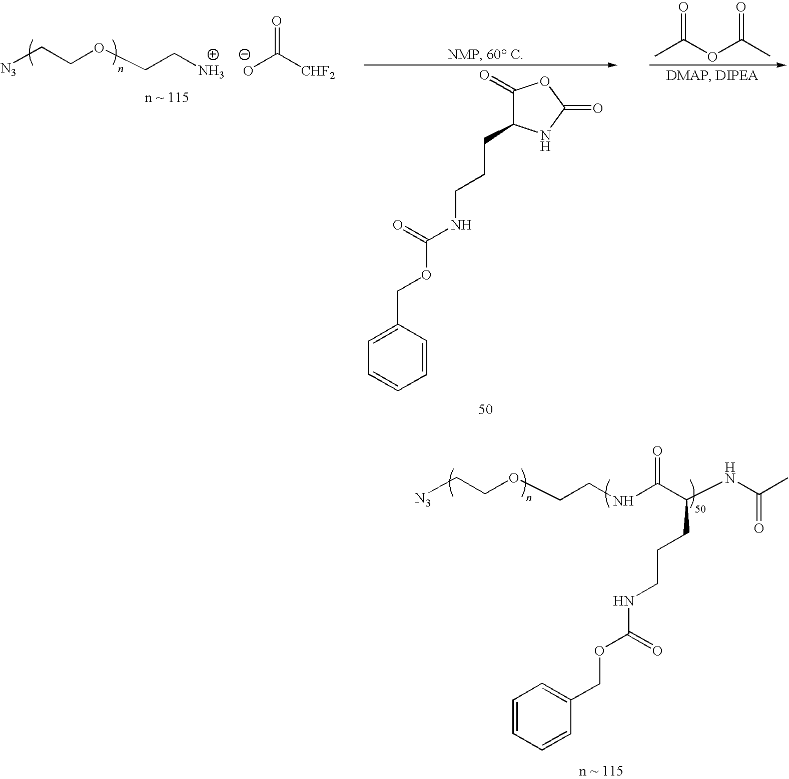

N₃-PEG5K-NH₂/DFA salt, (1 g, 0.2 mmol) was weighed into an oven-dried, round-bottom flask, dissolved in toluene, and dried by azeotropic distillation. Excess toluene was removed under vacuum. Orn(Z) NCA (2.92 g, 10 mmol) was added to the flask, the flask was evacuated under reduced pressure, and subsequently backfilled with nitrogen gas. Dry N-methylpyrrolidone (NMP) (20 mL) was introduced by syringe and the solution was heated to 60° C. The reaction mixture was allowed to stir for 4 days at 60° C. under nitrogen gas. The solution was cooled to room temperature and DIPEA (2.0 mL), DMAP (100 mg), and acetic anhydride (2.0 mL) were added. Stirring was continued for 1 hour at room temperature. The polymer was precipitated into diethyl ether (cooled down to −20° C.) and isolated by filtration. The product was isolated by filtration and dried in vacuo to give the block copolymer as an off-white powder. ¹H NMR (d₆-DMSO) δ 8.66-7.86, 7.48-6.99, 5.13-4.83, 4.3-3.78, 3.72-3.23, 3.14-2.86, 2.14-1.15 ppm

### Example 51

### Synthesis of N₃-PEG5K-b-P(Orn(Z)₁₀₀)-Ac

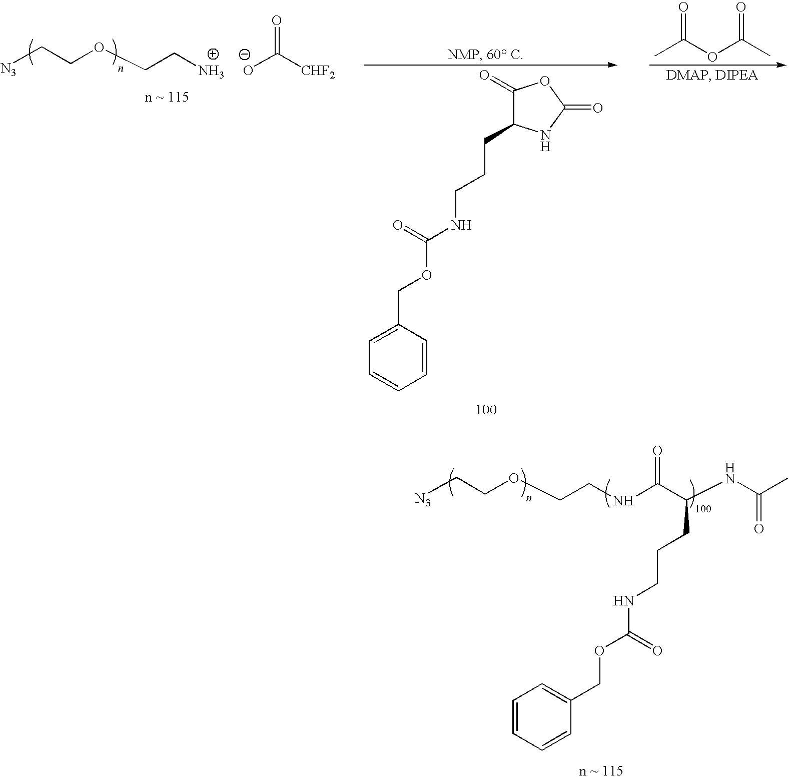

N₃-PEG5K-b-P(Orn(Z)₁₀₀)-Ac was synthesized as described in Example 50 from N₃-PEG-NH₂/DFA salt, 5 kDa (1 g, 0.2 mmol), Orn(Z)) NCA (5.85 g, 20 mmol) and 68 mL of NMP. The block copolymer was isolated as an off-white powder. ¹H NMR (d₆-DMSO) δ 8.66-7.86, 7.48-6.99, 5.13-4.83, 4.3-3.78, 3.72-3.23, 3.14-2.86, 2.14-1.15 ppm

### Example 52

### Synthesis of N₃-PEG5K-b-P(Asp(OBzl)₂₅-co-Asp(O^{t}Bu)₂₅)-Ac

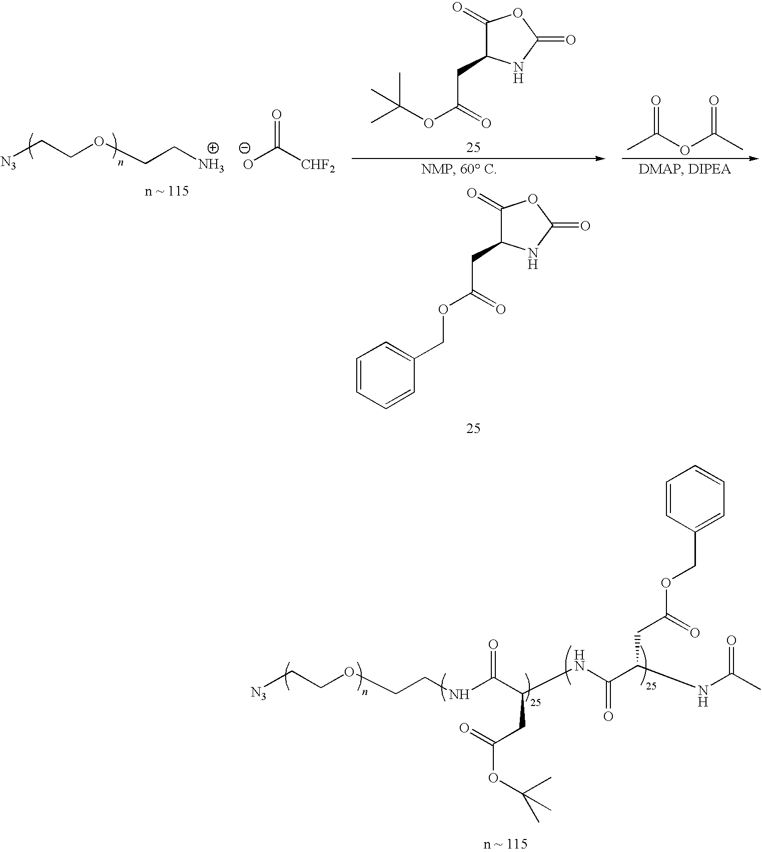

N₃-PEG5K-b-P(Asp(OBzl)₂₅-co-D-Asp(^{t}Bu)₂₅)-Ac was synthesized as described in Example 44 from N₃-PEG-NH₂/DFA salt, 5 kDa (1 g, 0.2 mmol), Asp(O^{t}Bu) NCA (1.25 g, 5 mmol), D-Asp(O^{t}Bu) NCA (1.08 g, 5 mmol) and 17 mL of NMP. The block copolymer was isolated as an off-white powder (1.81 g, 63% yield). ¹H NMR (d₆-DMSO) δ 8.50-7.67, 7.48-7.14, 5.18-4.91, 4.73-4.45, 3.71-3.38, 2.90-2.22, 1.52-1.12 ppm

### Example 53

### Synthesis of N₃-PEG5K-b-P(Asp(OBzl)₅₀-co-Asp(O^{t}Bu)₅₀)-Ac

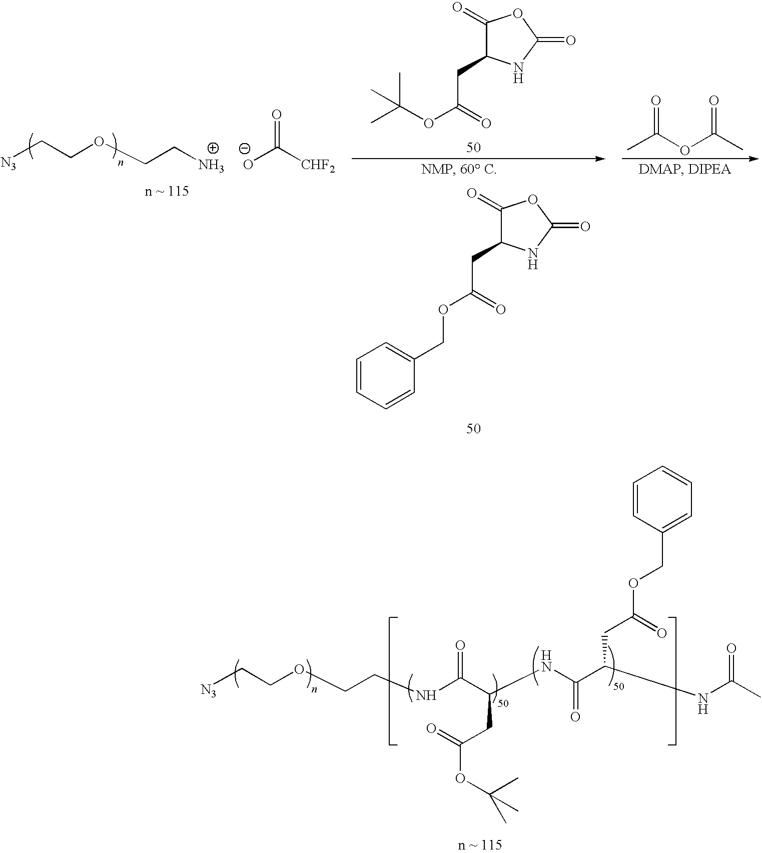

N₃-PEG5K-b-P(Asp(OBzl)₂₅-co-D-Asp(^{t}Bu)₂₅)-Ac was synthesized as described in Example 44 from N₃-PEG-NH₂/DFA salt, 5 kDa (1 g, 0.2 mmol), Asp(O^{t}Bu) NCA (2.49 g, 10 mmol), D-Asp(O^{t}Bu) NCA (2.15 g, 10 mmol) and 60 mL of NMP. The block copolymer was isolated as an off-white powder (2.74 g, 57% yield). ¹H NMR (d₆-DMSO) δ 8.50-7.67, 7.48-7.14, 5.18-4.91, 4.73-4.45, 3.71-3.38, 2.90-2.22, 1.52-1.12 ppm

### Example 54

### Synthesis of N₃-PEG12K-b-P(DLeu₂₀-co-Tyr(OBzl)₂₀)-Ac

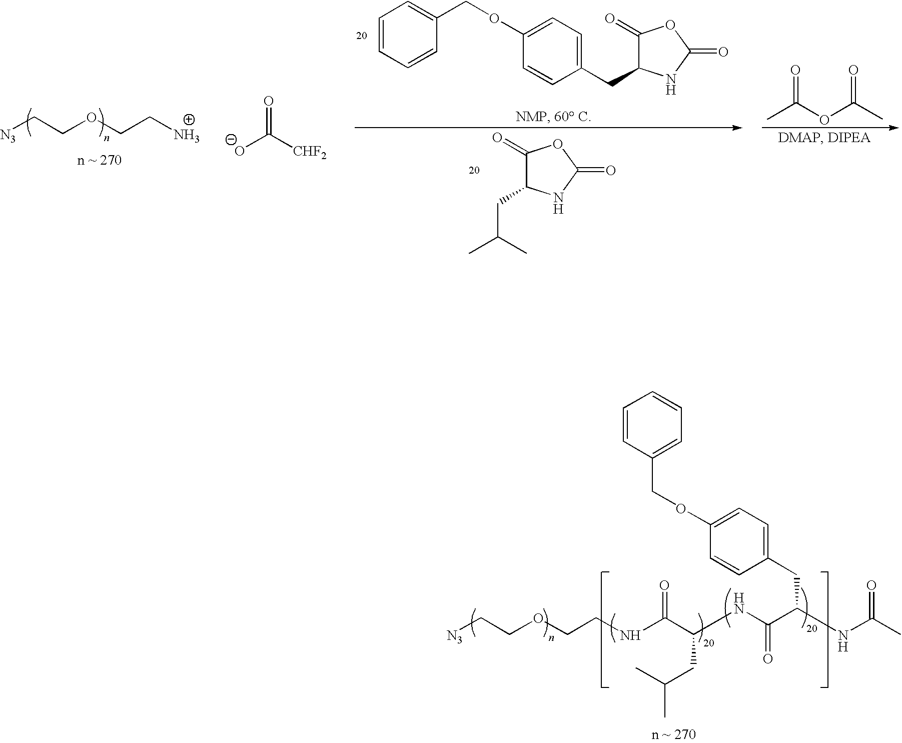

N₃-PEG12K-b-P(DLeu₂₀-co-Tyr(OBzl)₂₀)-Ac was synthesized as described in Example 31 from N₃-PEG-NH₂/DFA salt, 12 kDa (8 g, 0.66 mmol), D-Leu NCA (2.1 g, 13.4 mmol) Tyr(OBzl) NCA (3.96 g, 13.3 mmol) and 70 mL of NMP. The block copolymer was isolated as an off-white powder (9.85 g, 76% yield). ¹H NMR (d₆-DMSO) δ 8.44-7.69, 7.48-7.22, 7.19-7.02, 6.95-6.72, 5.08-4.83, 4.58-4.02, 3.70-3.41, 3.02-2.5, 1.60-0.50 ppm

### Example 55

### Synthesis of N₃-PEG12K-b-P(DLeu₂₀-co-Tyr(OBzl)₂₀)-Ac

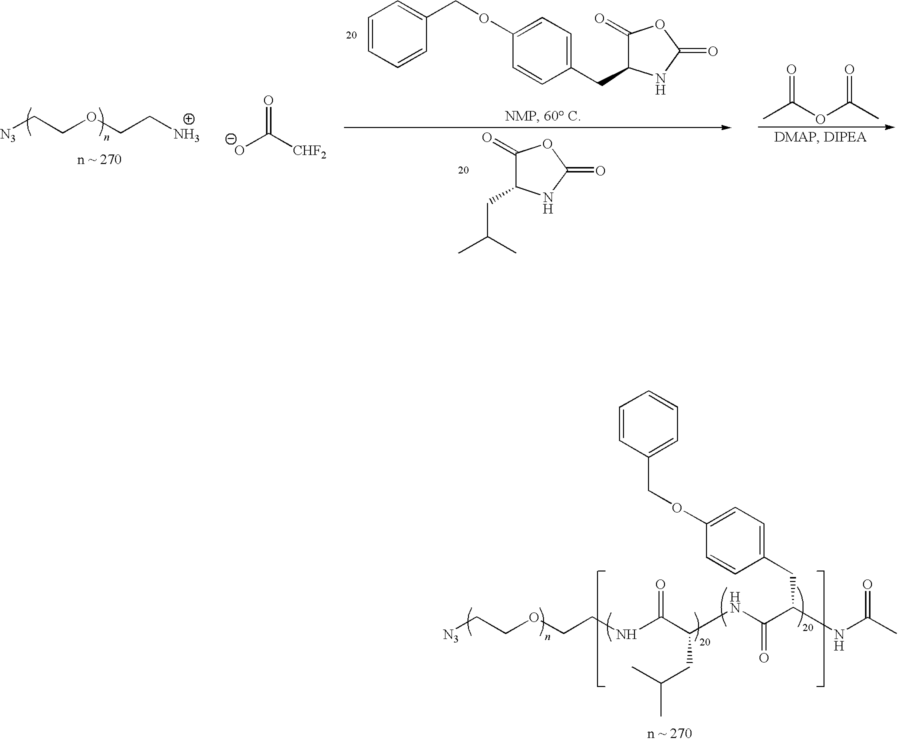

N₃-PEG12K-b-P(DLeu₃₀-co-Tyr(OBzl)₃₀)-Ac was synthesized as described in Example 31 from N₃-PEG-NH₂/DFA salt, 12 kDa (8 g, 0.66 mmol), D-Leu NCA (3.14 g, 20 mmol) Tyr(OBzl) NCA (5.95 g, 20 mmol) and 85 mL of NMP. The block copolymer was isolated as an off-white powder (10.46 g, 68% yield). ¹H NMR (d₆-DMSO) δ 8.44-7.69, 7.48-7.22, 7.19-7.02, 6.95-6.72, 5.08-4.83, 4.58-4.02, 3.70-3.41, 3.02-2.5, 1.60-0.50 ppm

### Example 56

### Synthesis of N₃-PEG12K-b-P(DLeu₂₀-co-Asp(O^{t}Bu)₂₀)-Ac

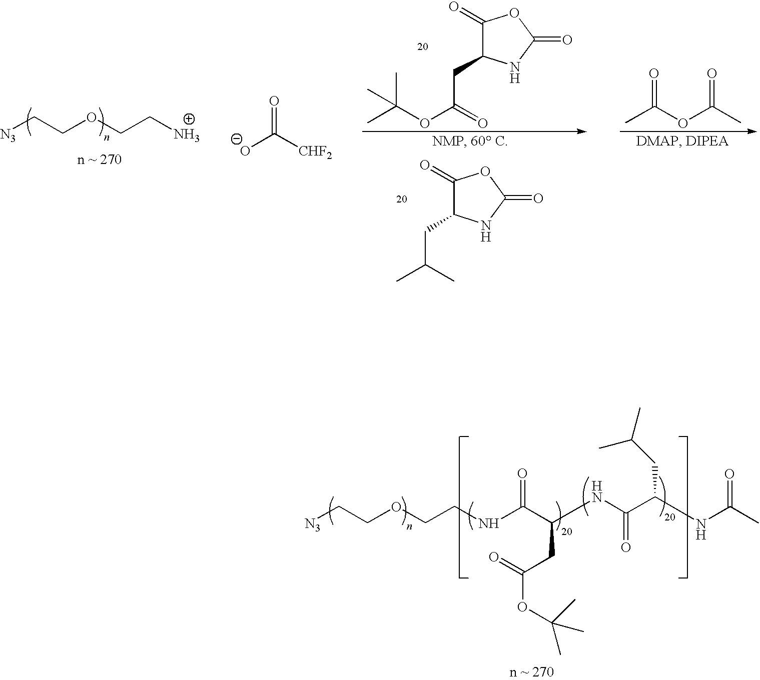

N₃-PEG12K-b-P(DLeu₂₀-co-Asp(O^{t}Bu)₂₀)-Ac was synthesized as described in Example 31 from N₃-PEG-NH₂/DFA salt, 12 kDa (8 g, 0.66 mmol), D-Leu NCA (2.1 g, 13.4 mmol) Asp(O^{t}Bu) NCA (2.87 g, 13.4 mmol) and 65 mL of NMP. The block copolymer was isolated as an off-white powder (8 g, 68% yield). ¹H NMR (d₆-DMSO) δ 8.52-7.33, 4.45, 3.81-3.35, 1.69-1.30, 1.00-0.74 ppm

### Example 57

### Synthesis of N₃-PEG12K-b-P(DLeu₃₀-co-Asp(O^{t}Bu)₁₀)-Ac

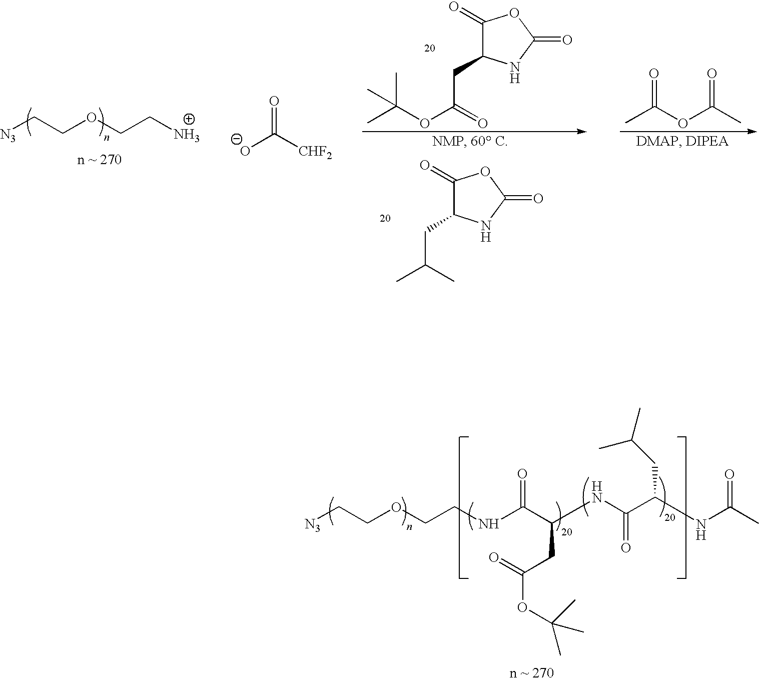

N₃-PEG12K-b-P(DLeu₂₀-co-Asp(O^{t}Bu)₂₀)-Ac was synthesized as described in Example 31 from N₃-PEG-NH₂/DFA salt, 12 kDa (8 g, 0.66 mmol), D-Leu NCA (3.14 g, 20 mmol) Asp(O^{t}Bu) NCA (1.44 g, 6.5 mmol) and 65 mL of NMP. The block copolymer was isolated as an off-white powder (7.96 g, 70% yield). ¹H NMR (d₆-DMSO) δ 8.52-7.33, 4.45, 3.81-3.35, 1.69-1.30, 1.00-0.74 ppm

### Example 58

### Synthesis of N₃-PEG12K-b-P(DLeu₂₀-co-Tyr₂₀)-Ac

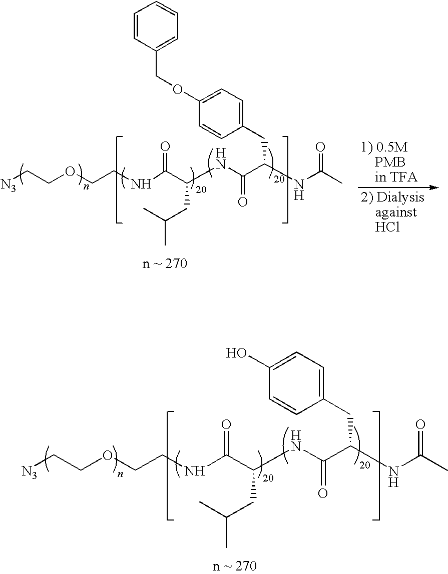

N₃-PEG12K-b-P(DLeu₂₀-co-Tyr(OBzl)₂₀)-Ac (9.5 g, 0.49 mmol) was dissolved in 100 mL of a 0.5 M solution of pentamethylbenzene (PMB) in trifluoroacetic acid (TFA). The reaction was allowed to stir for 3 hours at room temperature with a white precipitate forming after approximately 1 hour. The polymer was precipitated into diethyl ether (cooled down to −20° C.) and isolated by filtration. The product redissolved in dichloromethane, precipitated in cold ether (cooled down to −20° C.) and isolated by filtration and dried in vacuo to give the block copolymer as an off-white powder (8.4 g, 97% yield). ¹H NMR (d₆-DMSO) δ 9.29-8.93, 8.37-7.61, 7.09-6.86, 6.71-6.48, 4.52-3.96, 3.79-3.43, 2.99-2.73, 1.57-1.04, 1.04-0.50 ppm

### Example 59

### Synthesis of N₃-PEG12K-b-P(DLeu₃₀-co-Tyr₃₀)-Ac

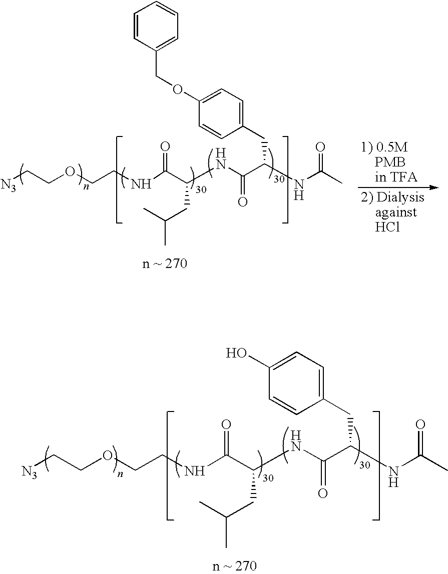

N₃-PEG12K-b-P(DLeu₂₀-co-Tyr(OBzl)₂₀)-Ac (9.5 g, 0.41 mmol) was dissolved in 100 mL of a 0.5 M solution of pentamethylbenzene (PMB) in trifluoroacetic acid (TFA). The reaction was allowed to stir for 3 hours at room temperature with a white precipitate forming after approximately 1 hour. The polymer was precipitated into diethyl ether (cooled down to −20° C.) and isolated by filtration. The product redissolved in dichloromethane, precipitated in cold ether (cooled down to −20° C.) and isolated by filtration and dried in vacuo to give the block copolymer as an off-white powder (7.99 g, 95% yield). ¹H NMR (d₆-DMSO) δ 9.29-8.93, 8.37-7.61, 7.09-6.86, 6.71-6.48, 4.52-3.96, 3.79-3.43, 2.99-2.73, 1.57-1.04, 1.04-0.50 ppm

### Example 60

### Synthesis of N₃-PEG12K-b-P(Asp(OBzl)₉₀-co-DLeu₁₀)-Ac

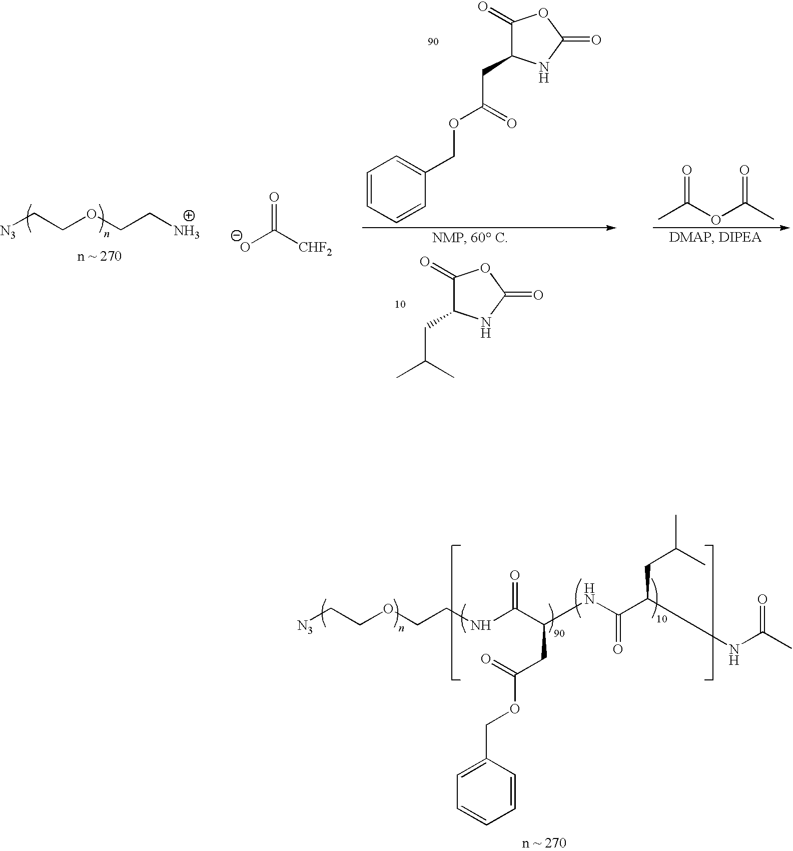

N₃-PEG12K-NH₂/DFA salt, (2 g, 0.17 mmol) was weighed into an oven-dried, round-bottom flask, dissolved in toluene, and dried by azeotropic distillation. Excess toluene was removed under vacuum. Asp(OBzl) NCA (3.90 g, 15.7 mmol) and D-Leu NCA (0.27 g, 1.74 mmol) was added to the flask, the flask was evacuated under reduced pressure, and subsequently backfilled with nitrogen gas (repeated twice). Dry N-methylpyrrolidone (NMP) (40 mL) was introduced by syringe and the solution was heated to 60° C. The reaction mixture was allowed to stir for 3 days at 60° C. under nitrogen gas. The solution was cooled to room temperature and DIPEA (2.0 mL), DMAP (200 mg), and acetic anhydride (2.0 mL) were added. Stirring was continued for 1 hour at room temperature. The polymer was then placed in a 3500 g/mol molecular weight cut-off dialysis bag, dialyzed three times against 0.1N HCl in methanol, three times against deionized water and freeze-dried. A white solid was obtained (2.441 g, 45% yield). ¹H NMR (d6-DMSO) δ 8.43-8.07, 7.45-7.16, 5.01, 4.61, 4.3-4.1, 3.68-3.38, 2.94-2.75, 2.75-2.5, 1.57-1.33, 0.84-0.63 ppm.

### Example 61

### Synthesis of N₃-PEG12K-b-P(Asp(OBzl)₇₀-co-DLeu₃₀)-Ac

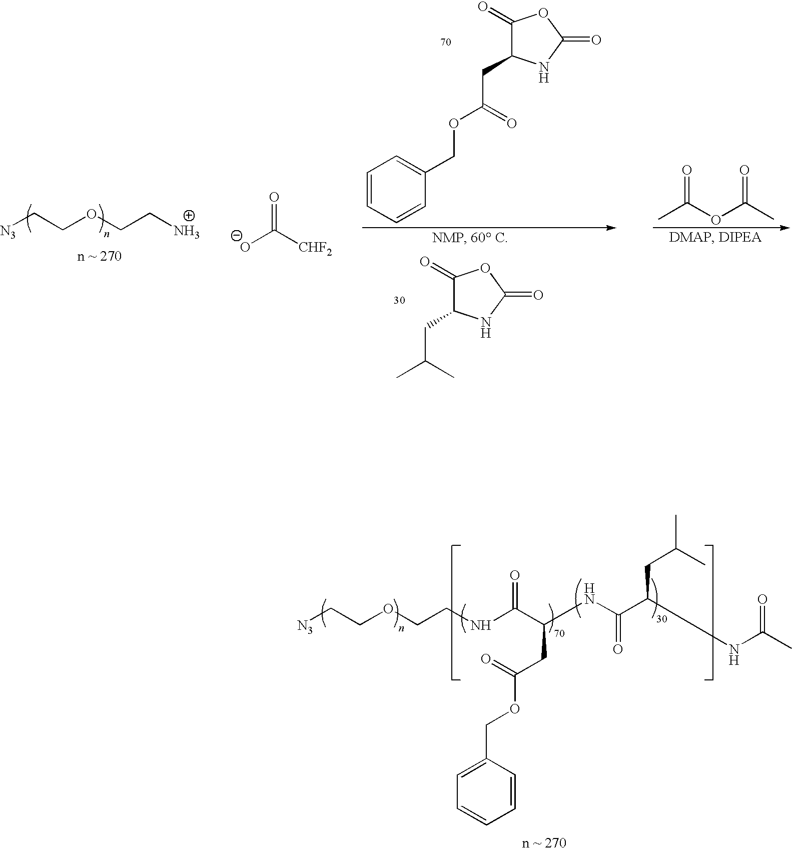

N₃-PEG12K-b-P(Asp(OBzl)₇₀-co-DLeu₃₀)-Ac was synthesized as described in Example 60 from N₃-PEG-NH₂/DFA salt, 12 kDa (2 g, 0.17 mmol), Asp(OBzl) NCA (3.03 g, 12.2 mmol), D-Leu NCA (0.82 g, 5.2 mmol) and 40 mL of NMP. The block copolymer was isolated as a white powder (3.395 g, 67% yield). ¹H NMR (d₆-DMSO) δ 8.43-8.07, 7.45-7.16, 5.01, 4.61, 4.3-4.1, 3.68-3.38, 2.94-2.75, 2.75-2.5, 1.57-1.33, 0.84-0.63 ppm

### Example 62

### Synthesis of N₃-PEG12K-b-P(Asp(OBzl)₅₀-co-DLeu₅₀)-Ac

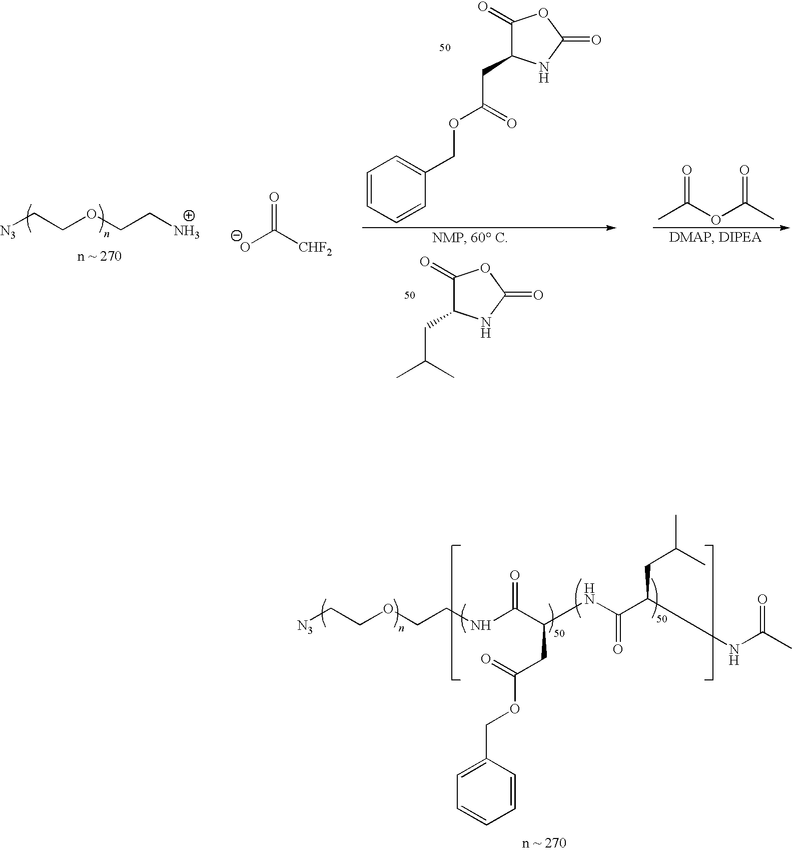

N₃-PEG12K-b-P(Asp(OBzl)₅₀-co-DLeu₅₀)-Ac was synthesized as described in Example 60 from N₃-PEG-NH₂/DFA salt, 12 kDa (2 g, 0.17 mmol), Asp(OBzl) NCA (2.17 g, 8.7 mmol), D-Leu NCA (1.37 g, 8.7 mmol) and 37 mL of NMP. The block copolymer was isolated as a white powder (2.887 g, 60.5% yield). ¹H NMR (d₆-DMSO) δ 8.43-8.07, 7.45-7.16, 5.01, 4.61, 4.3-4.1, 3.68-3.38, 2.94-2.75, 2.75-2.5, 1.57-1.33, 0.84-0.63 ppm

### Example 63

### Synthesis of N₃-PEG12K-b-P(Asp(OBzl)₁₈₀-co-DLeu₂₀)-Ac

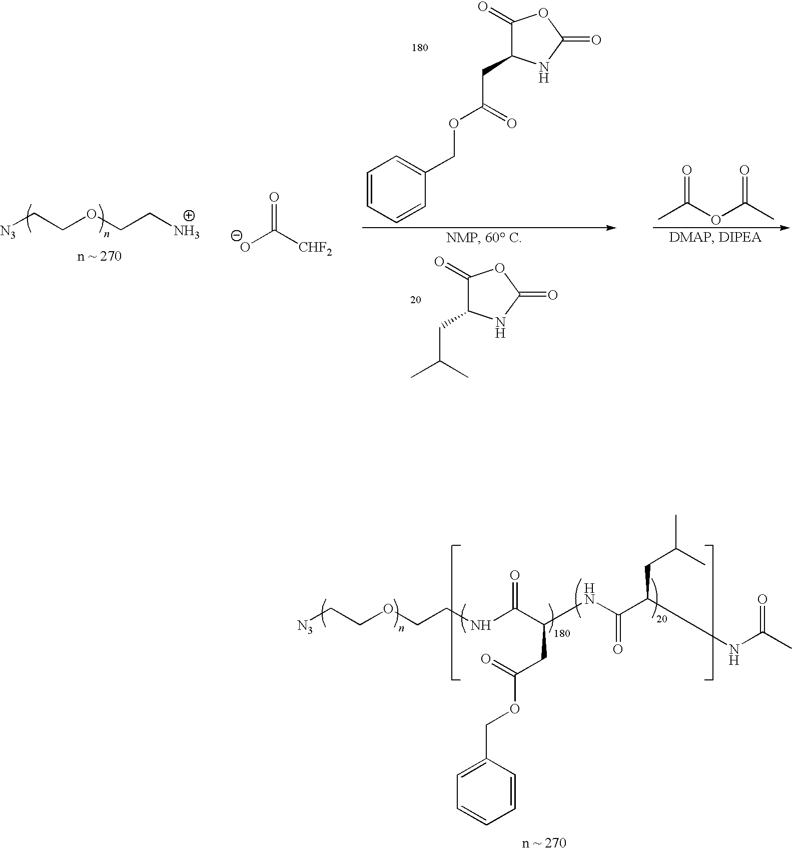

N₃-PEG12K-b-P(Asp(OBzl)₁₈₀-co-DLeu₂₀)-Ac was synthesized as described in Example 60 from N₃-PEG-NH₂/DFA salt, 12 kDa (1 g, 0.087 mmol), Asp(OBzl) NCA (3.90 g, 15.6 mmol), D-Leu NCA (0.27 g, 17.4 mmol) and 35 mL of NMP. The block copolymer was isolated as a white powder (1.685 g, 38% yield). ¹H NMR (d₆-DMSO) δ 8.43-8.07, 7.45-7.16, 5.01, 4.61, 4.3-4.1, 3.68-3.38, 2.94-2.75, 2.75-2.5, 1.57-1.33, 0.84-0.63 ppm

### Example 63

### Synthesis of N₃-PEG12K-b-P(Asp(OBzl)₁₄₀-co-DLeu₆₀)-Ac

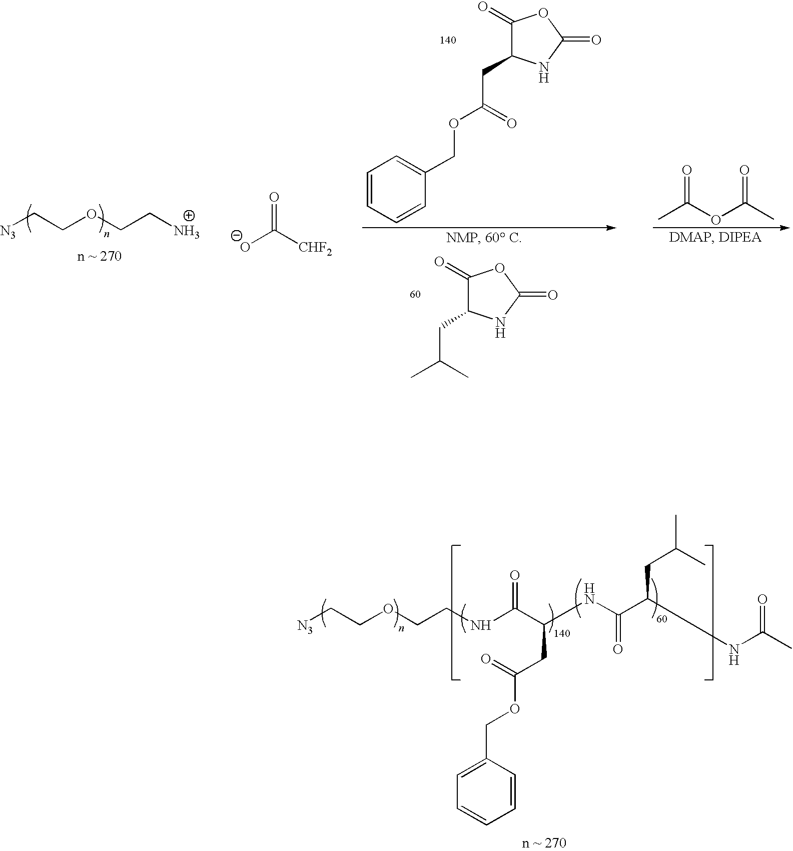

N₃-PEG12K-b-P(Asp(OBzl)₁₄₀-co-DLeu₆₀)-Ac was synthesized as described in Example 60 from N₃-PEG-NH₂/DFA salt, 12 kDa (1 g, 0.087 mmol), Asp(OBzl) NCA (3.03 g, 12.2 mmol), D-Leu NCA (0.82 g, 5.2 mmol) and 40 mL of NMP. The block copolymer was isolated as a white powder (1.784 g, 44% yield). ¹H NMR (d₆-DMSO) δ 8.43-8.07, 7.45-7.16, 5.01, 4.61, 4.3-4.1, 3.68-3.38, 2.94-2.75, 2.75-2.5, 1.57-1.33, 0.84-0.63 ppm

### Example 63

### Synthesis of N₃-PEG12K-b-P(Asp(OBzl)₁₀₀-co-DLeu₁₀₀)-Ac

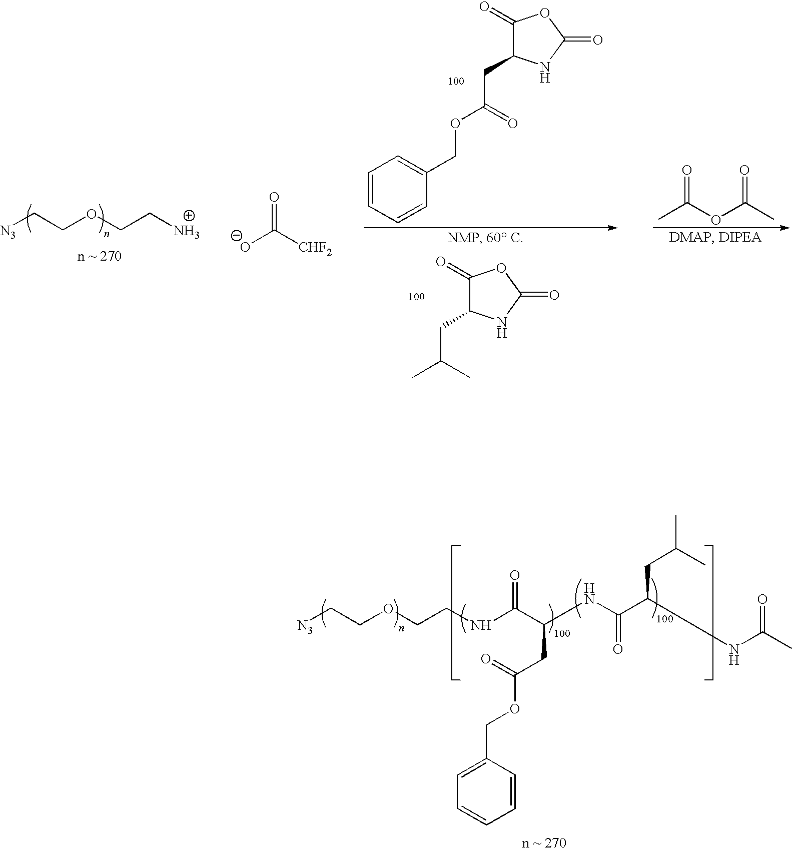

N₃-PEG12K-b-P(Asp(OBzl)₁₀₀-co-DLeu₁₀₀)-Ac was synthesized as described in Example 60 from N₃-PEG-NH₂/DFA salt, 12 kDa (1 g, 0.087 mmol), Asp(OBzl) NCA (2.17 g, 8.7 mmol), D-Leu NCA (1.37 g, 8.7 mmol) and 30 mL of NMP. The block copolymer was isolated as a white powder (2.792 g, 74% yield). ¹H NMR (d₆-DMSO) δ 8.43-8.07, 7.45-7.16, 5.01, 4.61, 4.3-4.1, 3.68-3.38, 2.94-2.75, 2.75-2.5, 1.57-1.33, 0.84-0.63 ppm

### Example 64

### Synthesis of N₃-PEG12K-b-P(Asp(OBzl)₁₉₀-co-DLeu₁₀)-Ac

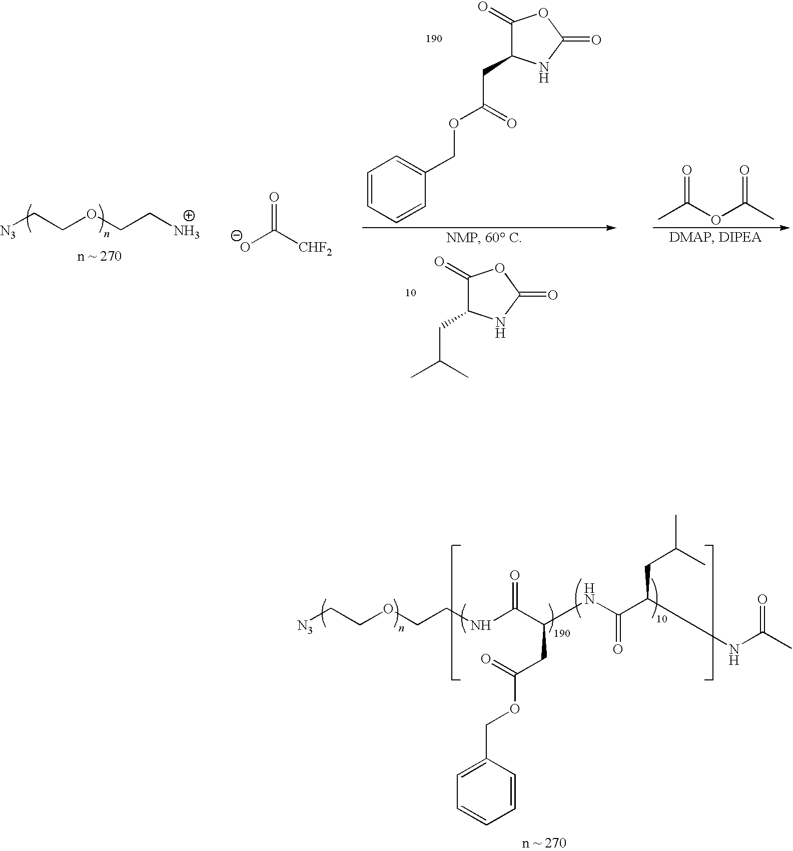

N₃-PEG12K-b-P(Asp(OBzl)₁₉₀-co-DLeu₁₀)-Ac was synthesized as described in Example 60 from N₃-PEG-NH₂/DFA salt, 12 kDa (1 g, 0.087 mmol), Asp(OBzl) NCA (4.12 g, 16.5 mmol), D-Leu NCA (0.14 g, 0.87 mmol) and 35 mL of NMP. The block copolymer was isolated as a white powder (1.83 g, 40.7% yield). ¹H NMR (d₆-DMSO) δ 8.43-8.07, 7.45-7.16, 5.01, 4.61, 4.3-4.1, 3.68-3.38, 2.94-2.75, 2.75-2.5, 1.57-1.33, 0.84-0.63 ppm

### Example 65

### Synthesis of N₃-PEG12K-b-P(Asp(OBzl)₁₇₀-co-DLeu₃₀)-Ac

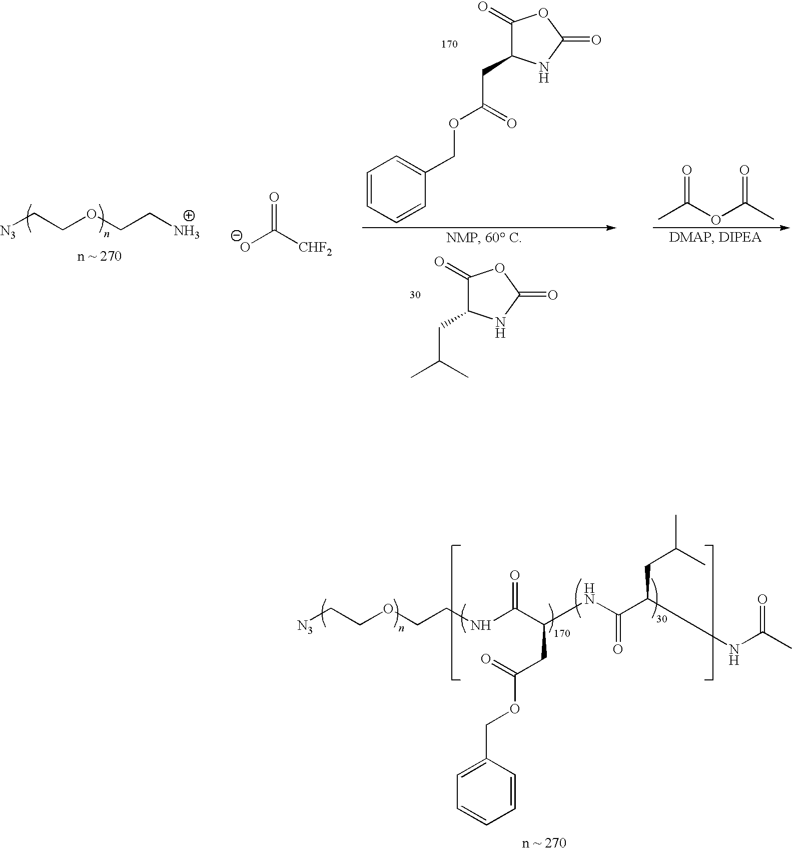

N₃-PEG12K-b-P(Asp(OBzl)₁₇₀-co-DLeu₃₀)-Ac was synthesized as described in Example 60 from N₃-PEG-NH₂/DFA salt, 12 kDa (1 g, 0.087 mmol), Asp(OBzl) NCA (3.68 g, 14.8 mmol), D-Leu NCA (0.41 g, 2.6 mmol) and 35 mL of NMP. The block copolymer was isolated as a white powder (1.38 g, 32% yield). ¹H NMR (d₆-DMSO) δ 8.43-8.07, 7.45-7.16, 5.01, 4.61, 4.3-4.1, 3.68-3.38, 2.94-2.75, 2.75-2.5, 1.57-1.33, 0.84-0.63 ppm

### Example 66

### Synthesis of N₃-PEG12K-b-P(Asp(OBzl)₁₅₀-co-DLeu₅₀)-Ac

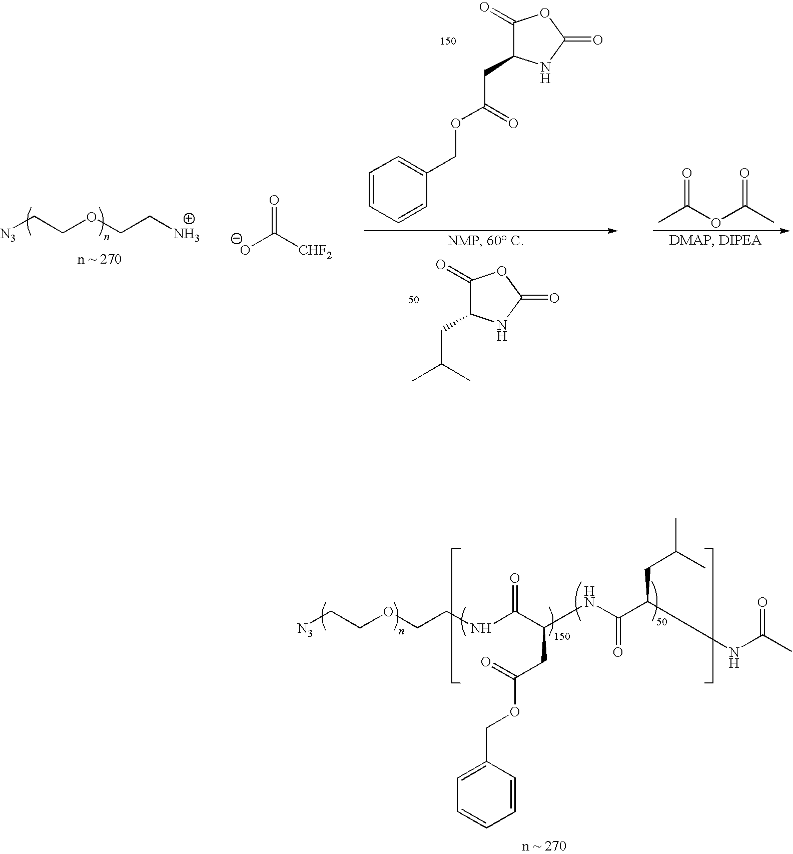

N₃-PEG12K-b-P(Asp(OBzl)₁₅₀-co-DLeu₅₀)-Ac was synthesized as described in Example 60 from N₃-PEG-NH₂/DFA salt, 12 kDa (1 g, 0.087 mmol), Asp(OBzl) NCA (3.25 g, 13 mmol), D-Leu NCA (0.68 g, 4.3 mmol) and 35 mL of NMP. The block copolymer was isolated as a white powder (1.82 g, 43.7% yield). ¹H NMR (d₆-DMSO) δ 8.43-8.07, 7.45-7.16, 5.01, 4.61, 4.3-4.1, 3.68-3.38, 2.94-2.75, 2.75-2.5, 1.57-1.33, 0.84-0.63 ppm

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of formula I: 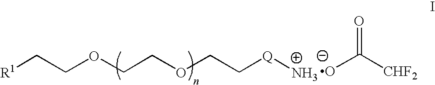
wherein:
n is 10-2500;
R¹ is -Z(CH₂CH₂Y)ₚ(CH₂)ₜR³, wherein:
Z is —O—, —S—, —C≡C—, or —CH₂—;
each Y is independently —O— or —S—;
p is 0-10;
t is 0-10; and
R³ is —N₃, —CN, a mono-protected amine, a di-protected amine, a protected aldehyde, a protected hydroxyl, a protected carboxylic acid, a protected thiol, a 9-30-membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety; and
Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

2. The compound according to claim 1, wherein R¹ is —N₃, —CH₃, or —C≡CH.

3. The compound according to claim 1, wherein R¹ is a mono-protected amine or a di-protected amine.

4. The compound according to claim 3, wherein R¹ is a mono-protected amine selected from t-butyloxycarbonylamino, ethyloxycarbonylamino, methyloxycarbonylamino, trichloroethyloxy-carbonylamino, allyloxycarbonylamino, benzyloxocarbonylamino, allylamino, benzylamino, fluorenylmethylcarbonyl, formamido, acetamido, chloroacetamido, dichloroacetamido, trichloroacetamido, phenylacetamido, trifluoroacetamido, benzamido, and t-butyldiphenylsilylamino.

5. The compound according to claim 3, wherein R¹ is a di-protected amine selected from di-benzylamine, di-allylamine, phthalimide, maleimide, succinimide, pyrrole, 2,2,5,5-tetramethyl-[1,2,5]azadisilolidine, and azide.

6. The compound according to claim 2, wherein Q is a valence bond.

7. The compound according to claim 1, wherein said compound is selected from:

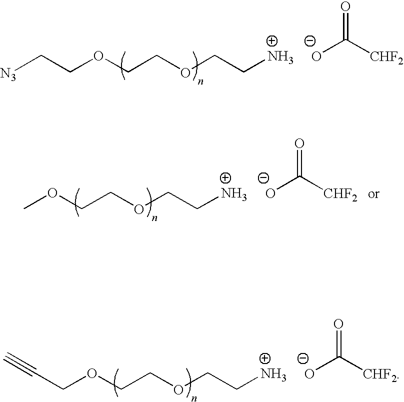

8. The compound according to claim 7, wherein each in is independently about 250 to about 300.

9. The compound according to claim 7, wherein each n is independently selected from 80±10, 115±10, 180±10, 225±10, 275±10, 315±10, or 340±10.

10. A method for preparing the compound according to claim 1 comprising the steps of:
(a) providing a compound of formula I-i: 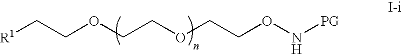
wherein PG is an acid-labile amino protecting group; and
(b) treating the compound of formula I-i with difluoroacetic acid to form the compound of formula I.

11. The method according to claim 10, wherein PG is tert-butyloxycarbonyl.

12. A method for preparing the compound according to claim 1, comprising the steps of:
(a) providing a compound of formula I-ii: 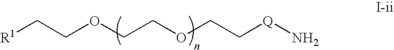
and
(b) treating the compound of formula I-ii with difluoroacetic acid to form the compound of formula I.

13. A compound of formula II: 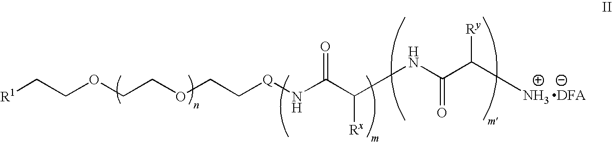
wherein:
n is 10-2500;
m is 0 to 1000;
m′ is 1 to 1000;
R^{x} is a natural or unnatural amino acid side-chain group that is capable of crosslinking;
R^{y} is a hydrophobic or ionic, natural or unnatural amino acid side-chain group;
R¹ is -Z(CH₂CH₂Y)ₚ(CH₂)ₜR³, wherein:
Z is —O—, —S—, —C≡C—, or —CH₂—;
each Y is independently —O— or —S—;
p is 0-10;
t is 0-10; and
R³ is hydrogen, —N₃, —CN, a mono-protected amine, a di-protected amine, a protected aldehyde, a protected hydroxyl, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety; and
Q is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
—Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

14. The compound according to claim 13, wherein R¹ is —N₃, —CH₃, or —C≡CH.

15. The compound according to claim 14, wherein n is selected from 80±10, 115±10, 180±10, 225±10, 275±10, 315±10, or 340±10.

16. The compound according to claim 13, wherein R^{x} is an amino acid side-chain group selected from tyrosine, serine, cysteine, threonine, aspartic acid, glutamic acid, asparagine, histidine, lysine, arginine, and glutamine.

17. The compound according to claim 16, wherein R^{y} is a hydrophobic amino acid side-chain group selected from D-leucine, D-phenylalanine, D-alanine, D-benzyl aspartate, or D-benzyl glutamate, and one or more of L-tyrosine, L-cysteine, L-aspartic acid, L-glutamic acid, L-DOPA, L-histidine, L-lysine, L-ornithine, or L-arginine, such that the overall R^{y} block is hydrophobic.

18. The compound according to claim 13, wherein m is 5-50 and m′ is 10-50.

19. A method for preparing the multi-block copolymer according to claim 13, wherein said method comprises the steps of:
(a) providing a compound of formula I: 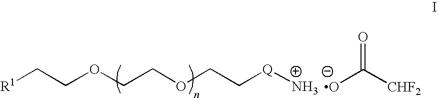
wherein:
n is 10-2500;
R¹ is -Z(CH₂CH₂Y)ₚ(CH₂)ₜR³, wherein:
Z is —O—, —S—, —C≡C—, or —CH₂—;
each Y is independently —O— or —S—;
p is 0-10;
t is 0-10; and
R³ is —N₃, —CN, a mono-protected amine, a di-protected amine, a protected aldehyde, a protected hydroxyl, a protected carboxylic acid, a protected thiol, a 9-30-membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety; and
Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
b) polymerizing a first cyclic amino acid monomer onto the amine salt terminal end of formula I, wherein said first cyclic amino acid monomer comprises R^{x}; and
(c) optionally polymerizing a second cyclic amino acid monomer, comprising R^{y}, onto the living polymer end, wherein said second cyclic amino acid monomer is different from said first cyclic amino acid monomer.

20. The method according to claim 19, further comprising the step of treating the compound of formula II with a suitable terminating agent to form a compound of formula III: 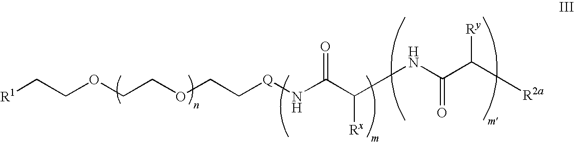
wherein:
n is 10-2500;
m is 0 to 1000;
m′ is 1 to 1000;
R^{x} is a natural or unnatural amino acid side-chain group that is capable of crosslinking;
R^{y} is a hydrophobic or ionic, natural or unnatural amino acid side-chain group;
R¹ is -Z(CH₂CH₂Y)ₚ(CH₂)ₜR³, wherein:
Z is —O—, —S—, —C≡C—, or —CH₂—;
each Y is independently —O— or —S—;
p is 0-10;
t is 0-10; and
R³ is hydrogen, —N₃, —CN, a mono-protected amine, a di-protected amine, a protected aldehyde, a protected hydroxyl, a protected carboxylic acid, a protected thiol, a 9-30 membered crown ether, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
Q is a valence bond or a bivalent, saturated or unsaturated, straight or branched C₁₋₁₂ hydrocarbon chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
R^{2a} is a mono-protected amine, a di-protected amine, —N(R⁴)₂, —NR⁴C(O)R⁴, —NR⁴C(O)N(R⁴)₂, —NR⁴C(O)OR⁴, or —NR⁴SO₂R⁴; and
each R⁴ is independently an optionally substituted group selected from hydrogen, aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
two R⁴ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur.
